SEQUENCE LISTING (14 sequences)

SEQ ID NO: 1 - protein, 1277 aa, Artificial Sequence
  Description of Artificial Sequence Synthetic polypeptide
MDWTWILFLVAAATRVHSSQCVNFTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3837 bp, Artificial Sequence
  Description of Artificial Sequence Synthetic polynucleotide
atggattggacttggattctctttctcgttgctgcagccacacgcgttcatagcagccagtgtgtgaacttcaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatctctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgtgctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaacattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtgaagggcttcaactgctacttccctctgcagagctacggcttccagccaacatatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaaggagtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagccacagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggaccctccagaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataa

SEQ ID NO: 3 - DNA, 6791 bp, Artificial Sequence
  Description of Artificial Sequence Synthetic polynucleotide
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctctttctcgttgctgcagccacacgcgttcatagcagccagtgtgtgaacttcaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatctctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgtgctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaacattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtgaagggcttcaactgctacttccctctgcagagctacggcttccagccaacatatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaaggagtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagccacagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggaccctccagaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 4 - DNA, 6797 bp, Artificial Sequence
  Description of Artificial Sequence Synthetic polynucleotide
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctctttctcgttgctgcagccacacgcgttcatagcagccagtgtgtgaacctgaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgacaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagagacctgcctcagggcttctctgccctggagcctctggtggacctgcccatcggcatcaacatcaccagattccagaccctgctggccctgcacagaagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaagattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtggaaggcttcaactgctacttccctctgcagagctacggcttccagccaacaaatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaagatgtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagctgagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 5 - protein, 1276 aa, Artificial Sequence
  Description of Artificial Sequence Synthetic polypeptide
MDWTWILFLVAAATRVHSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - DNA, 3834 bp, Artificial Sequence
  Description of Artificial Sequence Synthetic polynucleotide
atggattggacttggattctctttctcgttgctgcagccacacgcgttcatagcagccagtgtgtgaactttaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgctaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagaggcctgcctcagggcttctctgccctggagcctctggtggacctgcccatcggcatcaacatcaccagattccagaccctgcacatcagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaacattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtgaagggcttcaactgctacttccctctgcagagctacggcttccagccaacatatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaaggagtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagtggagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataa

SEQ ID NO: 7 - DNA, 6788 bp, Artificial Sequence
  Description of Artificial Sequence Synthetic polynucleotide
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctctttctcgttgctgcagccacacgcgttcatagcagccagtgtgtgaactttaccaccagaacacagctgcctcctgcctacaccaacagcttcaccagaggagtctactacccagacaaagtcttcagaagctctgtgctgcacagcacccaggacctgttcctgcctttcttcagcaacgtgacctggttccacgccatccacgtgtctggcaccaacggcaccaagagatttgctaaccctgttcttcctttcaatgatggcgtgtactttgccagcacagagaagagcaacatcatccgaggctggatctttggcaccaccctggacagcaaaacccagagcctgctgatcgtgaacaacgccaccaacgtggtcatcaaggtgtgtgagttccagttctgcaatgaccctttcctgggcgtgtactaccacaagaacaacaagtcctggatggagtctgagttcagagtctacagctctgccaacaactgcacatttgaatatgtgtcccagcctttcctgatggacctggagggcaagcagggcaactttaagaacctgagagaatttgtgttcaagaacatcgatggctacttcaagatctacagcaagcacacacccatcaacctggtgagaggcctgcctcagggcttctctgccctggagcctctggtggacctgcccatcggcatcaacatcaccagattccagaccctgcacatcagctacctgaccccaggagacagcagcagcggctggacagctggagctgctgcctactacgtgggctacctgcagcccaggaccttcctgctgaagtacaacgaaaatggcaccatcacagatgctgttgactgtgccctggaccctcttagcgagaccaagtgcaccctgaagtccttcacagtggagaaaggcatctaccagaccagcaacttccgagtgcagccaacagagagcatcgtgagatttccaaacatcaccaacctgtgcccttttggagaagtcttcaatgccaccagatttgcttctgtgtacgcctggaacagaaaaagaatcagcaactgtgtggctgactactctgtgctgtacaactctgcctccttctccaccttcaagtgctatggagtctctccaaccaagctgaatgacctgtgcttcaccaacgtgtatgctgacagctttgtgatcagaggagatgaagtgcggcagattgctcctggccagacaggcaacattgctgactacaactacaagctgcctgatgacttcacaggctgtgtcatcgcctggaacagcaacaacctggacagcaaggtgggcggcaactacaactacctgtacagacttttcaggaagagcaacctgaagccttttgaaagagacatctccacagagatctaccaggctggcagcacaccctgcaatggtgtgaagggcttcaactgctacttccctctgcagagctacggcttccagccaacatatggcgtgggctaccagccttacagagtggtggtgctgtcctttgagctgctgcacgcccctgccacagtgtgtggccccaagaagagcaccaacctggtgaagaacaaatgtgtgaacttcaatttcaatggcctgacaggcacaggagtgctgacagagagcaacaagaagtttcttcctttccagcagtttggaagagacattgctgacaccacagatgctgtgagagatcctcagaccctggagatcctggatatcacaccctgctcctttggaggagtttctgtcatcacacctggcaccaataccagcaaccaagtggctgtgctgtaccaaggagtgaattgcacagaagtgcctgtggccatccacgctgaccagctgacacccacctggagagtgtacagcacaggcagcaatgttttccagacaagagctggctgcctgattggagcagagcacgtgaacaacagctatgaatgtgacatccctattggagctggcatctgtgccagctaccagacccaaaccaacagcccaagaagagccagatctgtggccagccagagcatcatcgcctacaccatgagcctgggagtggagaactctgtggcctacagcaacaacagcatcgccatccccaccaacttcaccatctctgtgaccacagagatcctgcctgtgtccatgaccaagacatctgtggactgcaccatgtacatctgtggagacagcacagaatgcagcaacctgctgctgcagtacggctccttctgcacccagctgaacagagccctgacaggcatcgctgtggagcaggacaagaacacacaggaagtgtttgcccaggtgaagcagatctacaaaacaccacccatcaaggactttggaggcttcaatttctcccaaatcctgcctgaccccagcaagccttccaagagaagcttcattgaagacctgctgttcaacaaagtgaccctggctgatgctggcttcatcaagcagtatggagactgcctgggagacattgctgccagagacctgatctgtgcccagaagtttaatggcctgactgtgctgcctcctctgctgacagatgaaatgatcgcccagtacacatctgccctgctggctggcaccatcaccagtggctggacatttggagctggagctgccctgcagatcccttttgccatgcagatggcctacagatttaatggcatcggcgtgacccagaacgtgctgtacgagaaccagaagctgatcgccaaccagttcaactctgccatcggcaagatccaggacagcctgagcagcacagcctctgccctgggcaagctgcaggatgtggtgaaccaaaacgcccaggccctgaacaccctggtgaagcagctgagcagcaactttggagccatctcctctgtgctgaatgacatcctgagccggctggacaaggtggaagcagaagtgcagatcgacagactcatcacaggccgcctgcagagcctgcagacctacgtgacccagcagctgatcagagctgctgagatccgggcctctgccaacctggctgccaccaagatgtcagaatgtgtgctgggccagagcaaaagagtggacttctgtggcaaaggctaccacctgatgtccttccctcagtctgctcctcacggcgtggtgttcctgcacgtgacctacgtgcctgcccaggagaagaacttcaccacagctcctgccatctgccacgatggcaaggcccacttcccaagagaaggtgtctttgtgtccaatggcacccactggttcgtgacccagagaaacttctacgagcctcagatcatcaccacagacaacacatttgtgtctggcaactgtgatgtggtcatcggcatcgtgaacaacacagtttatgaccctctgcagcctgagctggacagcttcaaagaagagctggacaagtacttcaagaaccacacatctccagatgtggacctgggagacatctctggcatcaatgcctctgtggtgaacatccagaaggaaattgacaggctgaacgaagtggccaagaacctgaacgaaagcctcatcgacctgcaggagctgggcaagtacgagcagtacatcaagtggccttggtacatctggctgggcttcatcgctggcctcatcgccatcgtgatggtgaccatcatgctgtgctgcatgaccagctgctgctcttgcctgaagggctgctgcagctgtggcagctgctgcaagtttgatgaagatgactctgagcctgtgctgaagggcgtgaagctgcactacacatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 8 - DNA, 20 bp, Artificial Sequence
  Description of Artificial Sequence Synthetic primer
atgatcgcccagtacacatc

SEQ ID NO: 9 - DNA, 20 bp, Artificial Sequence
  Description of Artificial Sequence Synthetic primer
cacgccgatgccattaaatc

SEQ ID NO: 10 - DNA, 24 bp, Artificial Sequence
  Description of Artificial Sequence Synthetic probe
atcaccagtggctggacatttgga

SEQ ID NO: 11 - DNA, 20 bp, Artificial Sequence
  Description of Artificial Sequence Synthetic primer
gtgacgtggacatccgtaaa

SEQ ID NO: 12 - DNA, 21 bp, Artificial Sequence
  Description of Artificial Sequence Synthetic primer
cagggcagtaatctccttctg

SEQ ID NO: 13 - DNA, 24 bp, Artificial Sequence
  Description of Artificial Sequence Synthetic probe
taccctggcattgctgacaggatg

SEQ ID NO: 14 - DNA, 6 bp, Artificial Sequence
  Description of Artificial Sequence Synthetic oligonucleotide
gccacc

(19) United States
(12) Patent Application Publication
(10) Pub. No.: US 2022/0339280 A1
(43) Pub. Date: Oct. 27, 2022

(54) Immunogenic Compositions Against SARS-COV-2 Variants and Their Methods of Use

(71) Applicant: Inovio Pharmaceuticals, Inc., Plymouth Meeting, PA (US)

(72) Inventors: Charles Reed, Souderton, PA (US); Stephanie Ramos, Santee, CA (US); Trevor Smith, San Diego, CA (US); Maria Yang, San Diego, CA (US); Richa Kalia, San Diego, CA (US); Kate Broderick, San Diego, CA (US)

(21) Appl. No.: 17/726,952

(22) Filed: Apr. 22, 2022

Related U.S. Application Data
(60) Provisional application No. 63/178,849, filed on Apr. 23, 2021.
(60) Provisional application No. 63/182,501, filed on Apr. 30, 2021.
(60) Provisional application No. 63/208,545, filed on Jun. 9, 2021.
(60) Provisional application No. 63/212,345, filed on Jun. 18, 2021.
(60) Provisional application No. 63/227,774, filed on Jul. 30, 2021.
(60) Provisional application No. 63/237,262, filed on Aug. 26, 2021.
(60) Provisional application No. 63/248,072, filed on Sep. 24, 2021.
(60) Provisional application No. 63/225,827, filed on Jul. 26, 2021.
(60) Provisional application No. 63/309,357, filed on Feb. 11, 2022.

(51) Int. Cl. A61K 39/215; A61P 37/04

(52) U.S. Cl. CPC A61K 39/215; A61P 37/04; A61K 2039/53

(57) ABSTRACT

Disclosed herein are nucleic acid molecules encoding a Severe Acute Respiratory Syndrome coronavirus 2 (SARS-CoV-2) spike antigen, SARS-CoV-2 spike antigens, immunogenic compositions, and vaccines and their use in inducing immune responses and protecting against or treating a SARS-CoV-2 infection in a subject.

### CROSS-REFERENCE TO RELATED APPLICATIONS

The present application claims the benefit of U.S. Provisional Application 63/178,849, filed Apr. 23, 2021; U.S. Provisional Application 63/182,501, filed Apr. 30, 2021; U.S. Provisional Application 63/208,545, filed Jun. 9, 2021; U.S. Provisional Application 63/212,345, filed Jun. 18, 2021; U.S. Provisional Application 63/225,827, filed Jul. 26, 2021; U.S. Provisional Application 63/227,774, filed Jul. 30, 2021; U.S. Provisional Application 63/237,262, filed Aug. 26, 2021; U.S. Provisional Application 63/248,072, filed Sep. 24, 2021; and U.S. Provisional Application 63/309,357, filed Feb. 11, 2022. Each of these applications is incorporated by reference herein in its entirety.

### TECHNICAL FIELD

The present invention relates to vaccines for Severe Acute Respiratory Syndrome coronavirus 2 (SARS-CoV-2) and methods of administering the vaccines.

### BACKGROUND

Coronavirus Disease-19 (COVID-19) remains a global pandemic. To date, SARS-CoV-2 has infected over 500 million people and over 6 million people have succumbed to disease [World Health Organization. WHO Coronavirus (COVID-19) Dashboard. 2022]; Available from: https_covid19_who int]. Concerningly, Severe Acute Respiratory Syndrome coronavirus 2 (SARS-CoV-2) variants containing novel mutations impacting virological and epidemiological characteristics are driving an increased level of COVID-19 morbidity and mortality in many parts of the world.

Several variants have become a focus of attention. The B.1.1.7 (United Kingdom; Alpha), B.1.351 (South African; Beta)), P.1 (Brazilian; Gamma); B.1.617.2 (Delta); and B.1.1.529 (Omicron) variants have rapidly become dominant in some regions. Importantly, some of the mutations associated with these VOCs enhance resistance to neutralizing antibodies induced after infection or vaccination. [Collier, D. A., et al., Sensitivity of SARS-CoV-2 B.1.1.7 to mRNA vaccine-elicited antibodies. Nature, 2021; Garcia-Beltran, W. F., et al., Multiple SARS-CoV-2 variants escape neutralization by vaccine-induced humoral immunity. Cell, 2021; Wang, P., et al., Increased resistance of SARS-CoV-2 variant P.1 to Antibody Neutralization. Cell Host Microbe, 2021; Wang, Z., et al., mRNA vaccine-elicited antibodies to SARS-CoV-2 and circulating variants. Nature, 2021. 592(7855): p. 616-622; Wibmer, C. K., et al., SARS-CoV-2 501Y.V2 escapes neutralization by South African COVID-19 donor plasma. Nat Med, 2021. 27(4): p. 622-625; Liu, J., et al., BNT162b2-elicited neutralization of B.1.617 and other SARS-CoV-2 variants. Nature, 2021.].

Recent clinical data has revealed a significant decrease in efficacy of vaccines against novel mutations in the SARS-CoV-2 spike protein [Collier, D. A., et al., Sensitivity of SARS-CoV-2 B.1.1.7 to mRNA vaccine-elicited antibodies. Nature, 2021; Garcia-Beltran, W. F., et al., Multiple SARS-CoV-2 variants escape neutralization by vaccine-induced humoral immunity. Cell, 2021; Johnson & Johnson COVID-19 Vaccine Authorized by U.S. FDA For Emergency Use—First Single-Shot Vaccine in Fight Against Global Pandemic. 2021]. A recent Phase 3 clinical trial investigating the Vaxzevria vaccine demonstrated low efficacy (21.9%) against the circulating B.1.351 VOC in South Africa [Madhi, S. A., et al., Efficacy of the ChAdOx1 nCoV-19 Covid-19 Vaccine against the B.1.351 Variant. N Engl J Med, 2021]. Additionally, reports of fully vaccinated individuals suffering breakthrough infections with SARS-CoV-2 variants further highlight the risk variants pose [Hacisuleyman, E., et al., Vaccine Breakthrough Infections with SARS-CoV-2 Variants. N Engl J Med, 2021; Kustin, T., et al., Evidence for increased breakthrough rates of SARS-CoV-2 variants of concern in BNT162b2 mRNA vaccinated individuals. medRxiv, 2021: p. 2021 Apr. 06 21254882]. Pseudotyped virus and live virus neutralization results have shown reduced or in some cases loss of neutralizing antibody capacity against B.1.351 and B.1.617.2. Multiple growing lines of evidence are converging on the need to effectively address new variants and for strategies to refine vaccine designs for further emerging VOC challenges [Hodgson et al. What defines an efficacious COVID-19 vaccine? A review of the challenges assessing the clinical efficacy of vaccines against SARS-CoV-2. Lancet Infect Dis. 2021 February; 21(2):e26-e35. doi: 10.1016/S1473-3099(20)30773-8. Epub 2020 Oct. 27. PMID: 33125914; PMCID: PMC7837315; Gomez et al., Emerging SARS-CoV-2 Variants and Impact in Global Vaccination Programs against SARS-CoV-2/COVID-19. Vaccines (Basel). 2021 Mar. 11; 9(3):243. doi: 10.3390/vaccines9030243. PMID: 33799505.].

New vaccine candidates that address the challenge of current and emerging SARS-CoV-2 variants are needed.

### SUMMARY

Provided herein are nucleic acid molecules encoding a SARS-CoV-2 spike antigen. According to some embodiments, the encoded SARS-CoV-2 spike antigen is a consensus antigen. In some embodiments, the nucleic acid molecule comprises: the nucleic acid sequence of nucleotides 55 to 3831 of SEQ ID NO: 2; the nucleic acid sequence of SEQ ID NO: 2; or the nucleic acid sequence of SEQ ID NO: 3. Also provided herein are nucleic acid molecules encoding a SARS-CoV-2 spike antigen, wherein the SARS-CoV-2 spike antigen comprises: the amino acid sequence set forth in residues 19 to 1277 of SEQ ID NO: 1 or the amino acid sequence of SEQ ID NO: 1.

In some embodiments, the nucleic acid molecule encoding the SARS-CoV-2 antigen is incorporated into a viral particle.

Further provided are vectors comprising the nucleic acid molecule encoding the SARS-CoV-2 antigen. In some embodiments, the vector is an expression vector. The nucleic acid molecule may be operably linked to a regulatory element selected from a promoter and a poly-adenylation signal. The expression vector may be a plasmid or viral vector. An exemplary vector is pGX9527.

Immunogenic compositions comprising an effective amount of the vector or viral particle are disclosed. The immunogenic composition may comprise a pharmaceutically acceptable excipient, such as but not limited to, a buffer. The buffer may optionally be saline-sodium citrate buffer. In some embodiments, the immunogenic compositions comprise an adjuvant. An exemplary immunogenic composition is the INO-4802 drug product (or INO-4802 vaccine).

Also provided herein are SARS-CoV-2 spike antigens. According to some embodiments, the SARS-CoV-2 spike antigen is a consensus antigen. In some embodiments, the SARS-CoV-2 spike antigen comprises: the amino acid sequence set forth in residues 19 to 1277 of SEQ ID NO: 1 or the amino acid sequence of SEQ ID NO: 1.

Further provided herein are vaccines for the prevention or treatment of Severe Acute Respiratory Syndrome coronavirus 2 (SARS-CoV-2) infection. The SARS-CoV-2 infection prevented or treated in accordance with the invention includes the original Wuhan strain (WT), as well as variant strains including but not limited to variants of concern such as the B.1.1.7 (United Kingdom; Alpha) variant, the B.1.351 (South African; Beta) variant, the P.1 (Brazilian; Gamma) variant, the B.1.617.2 (Delta) variant, and the B.1.1.529 (Omicron) variant. The vaccines comprise an effective amount of any one or combination of the aforementioned nucleic acid molecules, vectors, or antigens. According to some embodiments, the vaccine further comprises a pharmaceutically acceptable excipient and/or adjuvant. The pharmaceutically acceptable excipient may be a buffer, optionally saline-sodium citrate buffer. According to some embodiments, the vaccine further comprises an adjuvant.

Methods of inducing an immune response against Severe Acute Respiratory Syndrome coronavirus 2 (SARS-CoV-2) in a subject in need thereof are further provided. In some embodiments, the methods of inducing an immune response comprise administering an effective amount of any one or combination of the aforementioned nucleic acid molecules, vectors, immunogenic compositions, antigens, or vaccines to the subject. Also provided herein are methods of protecting a subject in need thereof from infection with SARS-CoV-2, the method comprising administering an effective amount of any one or combination of the aforementioned nucleic acid molecules, vectors, immunogenic compositions, antigens, or vaccines to the subject. Further provided are methods of treating SARS-CoV-2 infection in a subject in need thereof, the method comprising administering an effective amount of any one or combination of the aforementioned nucleic acid molecules, vectors, immunogenic compositions, antigens, or vaccines to the subject. The SARS-CoV-2 infection prevented or treated in accordance with the invention includes the original Wuhan strain (WT), as well as variant strains including but not limited to variants of concern such as the B.1.1.7 (United Kingdom; Alpha) variant, the B.1.351 (South African; Beta) variant, the P.1 (Brazilian; Gamma) variant, the B.1.617.2 (Delta) variant, and the B.1.1.529 (Omicron) variant. Also provided herein are methods for treating or protecting a subject in need thereof against a disease or disorder associated with SARS-CoV-2 infection, the method comprising administering an effective amount of any one or combination of the aforementioned nucleic acid molecules, vectors, immunogenic compositions, antigens, or vaccines to the subject. In some embodiments, the disease or disorder associated with SARS-CoV-2 infection is Coronavirus Disease 2019 (COVID-19), Multisystem inflammatory syndrome in adults (MIS-A), or Multisystem inflammatory syndrome in children (MIS-C). In any of these methods, the administering may include at least one of electroporation and injection. According to some embodiments, the administering comprises parenteral administration, for example by intradermal, intramuscular, or subcutaneous injection, optionally followed by electroporation. In some embodiments of the disclosed methods, an initial dose of about 0.5 mg to about 2.0 mg of the nucleic acid molecule is administered to the subject, optionally the initial dose is 0.5 mg, 1.0 mg or 2.0 mg of the nucleic acid molecule. The methods may further involve administration of a subsequent dose of about 0.5 mg to about 2.0 mg of the nucleic acid molecule to the subject about four weeks after the initial dose, optionally wherein the subsequent dose is 0.5 mg, 1.0 mg or 2.0 mg of the nucleic acid molecule. In still further embodiments, the methods involve administration of one or more further subsequent doses of about 0.5 mg to about 2.0 mg of the nucleic acid molecule to the subject at least twelve weeks after the initial dose, optionally wherein the further subsequent dose is 0.5 mg, 1.0 mg, or 2.0 mg of the nucleic acid molecule. According to some embodiments, the nucleic acid molecule comprises: the nucleic acid sequence of nucleotides 55 to 3831 of SEQ ID NO: 2; the nucleic acid sequence of SEQ ID NO: 2; the nucleic acid sequence of SEQ ID NO: 3; or a nucleic acid molecule encoding a SARS-CoV-2 spike antigen, wherein the SARS-CoV-2 spike antigen comprises the amino acid sequence set forth in residues 19 to 1277 of SEQ ID NO: 1 or the amino acid sequence of SEQ ID NO: 1. For example, pGX9527, INO-4802 drug product, or a biosimilar thereof may be administered in accordance with any of the aforementioned methods.

Also provided herein are uses of any one or combination of the disclosed nucleic acid molecules, vectors, immunogenic compositions, antigens, or vaccines in a method of inducing an immune response against Severe Acute Respiratory Syndrome coronavirus 2 (SARS-CoV-2) in a subject in need thereof. Further provided are uses of any one or combination of the disclosed nucleic acid molecules, vectors, immunogenic compositions, antigens, or vaccines in a method of protecting a subject from infection with Severe Acute Respiratory Syndrome coronavirus 2 (SARS-CoV-2). Also provided herein are uses of any one or combination of the disclosed nucleic acid molecules, vectors, immunogenic compositions, antigens, or vaccines in a method of treating a subject in need thereof against SARS-CoV-2 infection. The SARS-CoV-2 infection prevented or treated in accordance with the invention includes the original Wuhan strain (WT), as well as variant strains including but not limited to variants of concern such as the B.1.1.7 (United Kingdom; Alpha) variant, the B.1.351 (South African; Beta) variant, the P.1 (Brazilian; Gamma) variant, the B.1.617.2 (Delta) variant, and the B.1.1.529 (Omicron) variant. Also provided herein are uses of any one or combination of the disclosed nucleic acid molecules, vectors, immunogenic compositions, antigens, or vaccines in a method of treating or protecting a subject in need thereof against a disease or disorder associated with SARS-CoV-2 infection. In some embodiments, the disease or disorder associated with SARS-CoV-2 infection is Coronavirus Disease 2019 (COVID-19), Multisystem inflammatory syndrome in adults (MIS-A), or Multisystem inflammatory syndrome in children (MIS-C). In accordance with any of these uses, the nucleic acid molecule, the vector, the immunogenic composition, the antigen, or the vaccine may be administered to the subject by at least one of electroporation and injection. In some embodiments, the nucleic acid molecule, the vector, the immunogenic composition, the antigen, or the vaccine is administered parenterally to the subject followed by electroporation. In some embodiments of the disclosed uses, an initial dose of about 0.5 mg to about 2.0 mg of the nucleic acid molecule is administered to the subject, optionally the initial dose is 0.5 mg, 1.0 mg or 2.0 mg of the nucleic acid molecule. The uses may further involve administration of a subsequent dose of about 0.5 mg to about 2.0 mg of the nucleic acid molecule to the subject about four weeks after the initial dose, optionally wherein the subsequent dose is 0.5 mg, 1.0 mg or 2.0 mg of the nucleic acid molecule. In still further embodiments, the uses involve administration of one or more further subsequent doses of about 0.5 mg to about 2.0 mg of the nucleic acid molecule to the subject at least twelve weeks after the initial dose, optionally wherein the further subsequent dose is 0.5 mg, 1.0 mg, or 2.0 mg of the nucleic acid molecule. According to some embodiments, the nucleic acid molecule administered in accordance with any of the aforementioned uses comprises: the nucleic acid sequence of nucleotides 55 to 3831 of SEQ ID NO: 2; the nucleic acid sequence of SEQ ID NO: 2; the nucleic acid sequence of SEQ ID NO: 3; or a nucleic acid molecule encoding a SARS-CoV-2 spike antigen, wherein the SARS-CoV-2 spike antigen comprises the amino acid sequence set forth in residues 19 to 1277 of SEQ ID NO: 1 or the amino acid sequence of SEQ ID NO: 1. For example, pGX9527, INO-4802 drug product or a biosimilar thereof may be administered in accordance with any of the aforementioned uses.

Further provided herein are uses of any one or combination of the disclosed nucleic acid molecules, vectors, immunogenic compositions, antigens, or vaccines in the preparation of a medicament. In some embodiments, the medicament is for treating or protecting against infection with Severe Acute Respiratory Syndrome coronavirus 2 (SARS-CoV-2). The SARS-CoV-2 infection prevented or treated in accordance with the invention includes the original Wuhan strain (WT), as well as variant strains including but not limited to variants of concern such as the B.1.1.7 (United Kingdom; Alpha) variant, the B.1.351 (South African; Beta) variant, the P.1 (Brazilian; Gamma) variant, the B.1.617.2 (Delta) variant, and the B.1.1.529 (Omicron) variant. In some embodiments, the medicament is for treating or protecting against a disease or disorder associated with SARS-CoV-2 infection. In some embodiments, the medicament is for treating or protecting against Coronavirus Disease 2019 (COVID-19), Multisystem inflammatory syndrome in adults (MIS-A), or Multisystem inflammatory syndrome in children (MIS-C). According to some embodiments, the nucleic acid molecule administered in accordance with any of the aforementioned uses comprises: the nucleic acid sequence of nucleotides 55 to 3831 of SEQ ID NO: 2; the nucleic acid sequence of SEQ ID NO: 2; the nucleic acid sequence of SEQ ID NO: 3; or a nucleic acid molecule encoding a SARS-CoV-2 spike antigen, wherein the SARS-CoV-2 spike antigen comprises the amino acid sequence set forth in residues 19 to 1277 of SEQ ID NO: 1 or the amino acid sequence of SEQ ID NO: 1. For example, pGX9527, INO-4802 drug product or a biosimilar thereof may be administered in accordance with any of the aforementioned uses.

### BRIEF DESCRIPTION OF THE DRAWINGS

FIGS. 1A-1J detail design strategy of a pan-SARS-CoV-2 vaccine pGX9527. As shown in FIG. 1A, SARS-CoV-2 spike glycoprotein sequences were sampled from multiple countries (Brazil, Canada, India, Italy, Japan, Nigeria, South Africa, the United Kingdom, and the United States) and the most prevalent mutations were aggregated for each location. The regional mutations were then analyzed and aggregated to generate the SynCon® spike antigen comprising amino acids 19-1277 of SEQ ID NO: 1. Lastly, in addition to the global SynCon® changes to the spike, supplemental variant of concern (VOC) changes were placed in the receptor binding domain (RBD) and the 2P mutation was added producing pGX9527 spike antigen insert. FIG. 1B illustrates an unrooted phylogenetic tree comparing protein sequences derived from pGX9527 (the plasmid included in the INO-4802 drug product), pB.1.351, and pWT spike antigens as well as spike sequences from a sampling of circulating variants including current VOCs. FIG. 1C identifies the Spike glycoprotein mutations in antigens from VOCs relative to wild-type (WT) Spike protein sequence (GenBank RefSeq sequence NC_045512.2 from Wuhan (China)). FIG. 1D provides a diagrammatic representation of the modified pVAX1 backbone (pGX0001). FIG. 1E illustrates the construction of plasmid pGX9527 (also referred to as pS-Pan). FIG. 1F provides descriptions of the disclosed plasmids. FIG. 1G shows analysis of in vitro expression of Spike protein after transfection of 293T cells with empty vector (pVax), pWT, pS-Pan (pGX9527, INO-4802), or pB.1.351 plasmid by Western blot. Control proteins and 293T cell lysates were resolved on a gel and probed with a polyclonal anti-SARS-CoV-2 Spike RBD Protein. Blots were stripped then probed with an anti-β-actin loading control. Bands were detected at the expected SARS-COV-2 Spike antigen molecular weight of about 180 kDa inclusive of glycosylation. FIG. 1H shows analysis of in vitro expression of Spike protein after transfection of 293T cells with empty vector (pVax), pS-WT, pS-Pan, or pS-B.1.351 plasmid by Western blot. Control proteins and 293T cell lysates were resolved on a gel and probed with a polyclonal anti-SARS-CoV-2 Spike RBD Protein. Blots were stripped then probed with an anti-β-actin loading control. FIG. 11 shows in vitro expression of RNA by RT-PCR assay. RNA extracts from COS-7 cells transfected in duplicate with pS-WT, pS-Pan, or pS-B.1.351. Extracted RNA was analyzed by RT-PCR using a PCR assays designed for SARS-CoV-2 spike and for COS-7 β-Actin mRNA, used as an internal expression normalization gene. Delta CT (A CT) was calculated as the CT of the target minus the CT of β-Actin for each transfection concentration and is plotted against the log of the mass of pDNA transfected (Plotted as mean±SD). FIG. 1J shows a molecular model of SARS-CoV-2 spike showing locations of mutations for INO-4802 colored similarly to FIG. 1A. For clarity a single spike subunit is labeled. Remaining subunits are indicated as transparent surfaces. Mutations not easily visible on a view are indicated with arrows and not all mutations are indicated in all views. Orthogonal views of the model are shown. Potential glycosylation sites are indicated in light green. The L18F mutation is not visualized in the model. The stalk region and membrane orientation are indicated by cartoon schematic.

FIGS. 2A-2D show pGX9527-induced humoral immune responses against SARS-CoV-2 VOC. BALB/c mice were immunized on days 0 and 14 with 10 μg of pWT (pGX9501; SEQ ID NO: 4), pB.1.351 (pGX9517; SEQ ID NO: 7), or pGX9527 (“INO-4802”; SEQ ID NO: 3) and sera samples were collected at day 21 for evaluation of antibody responses as described in the methods. FIG. 2A shows sera IgG binding titers against the indicated Spike proteins for pWT, pB.1.351, or pGX9527 (INO-4802)-vaccinated mice (n of 8 each). Data shown represent geometric mean titer values (GMT+/−95% CI) for each group of 8 mice. FIG. 2B shows sera pseudovirus neutralization ID₅₀ titers against the indicated SARS-CoV-2 variant for pWT, pB.1.351, or pGX9527 (INO-4802)-vaccinated mice (n of 8 per group) or human convalescent sera samples (n of 20). Each data point represents the mean of technical duplicates for individual samples. Dashed lines represent the limit of detection (LOD) of the assay. Samples below LOD were plotted at the number equivalent to half of the lowest serum dilution. *P<0.05, **P<0.005, ***P<0.001 determined by Kruskal-Wallis test (ANOVA) with Dunn multiple comparisons test. FIG. 2C shows IgG binding data represented as group means for each variant tested. FIG. 2D shows pseudovirus neutralization ID₅₀ titer data represented as group means for each variant tested.

FIGS. 3A-3G show pGX9527(INO-4802)-induced cellular immune response against SARS-CoV-2 variants. Splenocytes isolated from mice were collected 1 week after receiving the second dose of either pGX9501 (also referred to as pS-WT), pGX9517 (also referred to as pS-B.1.351), or pGX9527 (also referred to as pS-Pan or INO-4802). In FIG. 3A, splenocytes were stimulated with peptide pools spanning the entire Spike proteins of the WT, B.1.1.7, P.1, or B.1.351 variants and cellular responses were measured by IFNγ ELISpot assay. Mean+/−SD IFNγ SFUs/million splenocytes of experimental triplicates are shown. In FIGS. 3B-3E, intracellular cytokine staining was employed for CD4+ and CD8+ T cell activation. Expression levels of IFNγ, CD107a and IL-4 were analyzed. FIG. 3F is a representative graph showing correlation of T_{H}1 (IFNγ) versus T_{H}2 (IL-4) cytokine expression in the CD4 compartment of pGX9527-treated animals restimulated with either the WT, B.1.1.7, P.1, or B.1.351 peptide pools. FIG. 3G shows frequencies of circulating Tfh cells (CXCR5+PD-1+) in CD4 T cells 2 weeks after the second dose of either pS-WT or pS-Pan. *P<0.05, (Mann-Whitney test).

FIGS. 4A and 4B show that heterologous boost with pGX9527 induces humoral immune responses against SARS-CoV-2 variants in Syrian Hamsters. FIG. 4A shows experimental design. Syrian hamsters (n=8) were immunized with 90 μg of pGX9501 (pWT) on days 0 and 14. On day 236 animals were boosted with 90 μg of pGX9501 or pGX9527 (“INO-4802”). FIG. 4B shows pre- and post-boost sera IgG binding titers against the indicated SARS-CoV-2 Spike antigens. Symbols represent endpoint binding titers for individual animals and lines and bars represent GMT+/−95% CI. Values indicate log2 fold changes of GMTs from pre- to post-boost.

FIG. 5 illustrates the IgG isotype profile of pGX9527 humoral immunogenicity against SARS-CoV-2 VOC. BALB/c mice were immunized on days 0 and 14 with 10 μg of pWT or pGX9527as described in the methods. Protein antigen binding of IgG2A and IgG1 from mice at day 21 (7 days post second immunization). Data shown represent OD450 nm values for sera at a 1:1350 dilution (linear range of binding for all groups/protein antigens) for each group of mice. Protein antigens are SARS-CoV-2 full length spike proteins (circle—WT; square—B.1.1.7; triangle—P.1; and diamond—B.1.351) representing the full mutational profile of each VOC as described in the methods.

FIGS. 6A and 6B show in vitro expression of pDNA. FIG. 6A details analysis of in vitro expression of Spike protein after transfection of 293T cells with empty vector (pVax), pWT, pGX9527, or pB.1.351 plasmid by Western blot. Control proteins and 293T cell lysates were resolved on a gel and probed with a polyclonal anti-SARS-CoV-2 Spike RBD Protein. Blots were stripped then probed with an anti-β-actin loading control. FIG. 6B shows in vitro expression of RNA by RT-PCR assay. RNA extracts from COS-7 cells transfected in duplicate with pWT, pGX9527, or pB.1.351. Extracted RNA was analyzed by RT-PCR using a PCR assays designed for SARS-CoV-2 spike and for COS-7 β-Actin mRNA, used as an internal expression normalization gene. Delta CT (Δ CT) was calculated as the CT of the target minus the CT of β-Actin for each transfection concentration and is plotted against the log of the mass of pDNA transfected (Plotted as mean±SD).

FIGS. 7A-7E show that pGX9527 (“INO-4802”) protects Syrian Golden Hamsters against challenge with B.1.351 live virus. FIG. 7A shows a study schematic: 6 hamsters received ID+EP immunizations with 95 ug pWT, pB.1.351 or INO-4802 on days 0 and 22. Hamsters were challenged intranasally (IN) with 1.1×10\hat{ }5 PFU B.1.351 and observed for weight loss. On day 4 post challenge animals were euthanized and lung tissue was harvested for viral load measurement. Pre-challenge sera from pGX9527 (“INO-4802”)-immunized hamsters taken at the time of live virus challenge neutralized both WT (mean ID₅₀ 672.2) and B.1.351 pseudovirus in vitro (mean ID₅₀ 1121) (FIG. 7B). Weight change of pWT (left, n=6), pB.1.351 (center, n=6) or INO-4802 (right, n=5) vaccinated hamsters compared to unvaccinated animals (n=6) following challenge with B.1.351 live virus. All hamsters receiving two doses of pGX9527 (“INO-4802”) were protected from weight loss after B.1.351 live virus challenge (FIG. 7C). As shown in FIG. 7D, sera of vaccinated hamsters inhibits binding of the human host receptor ACE-2 to B.1.351 SARS-CoV-2 spike protein in vitro. Serum of hamsters vaccinated on days 0 and 22 with indicated SARS-CoV-2 vaccine constructs was collected on day 40 of the experiment. Serum was diluted 1:30 and tested for ACE-2 inhibition in an electrochemiluminescent-based ELISA assay. Depicted is percent inhibition of ACE-2 binding (mean % inhibition+/−SEM, n=6 or 4). As shown in FIG. 7E, viral loads in the lung at day 4 post challenge as shown by median tissue culture ID50 (TCID₅₀) per gram of lung tissue (mean+/−SEM). Animals with viral loads below the LOD of the assay are graphed at 1687 TCID₅₀/g, the lower limit of detection. *P<0.05, **P<0.005, ***P<0.001 determined by Mann-Whitney test.

FIG. 8 illustrates the correlation between pseudovirus and ACE2 blocking assays. Relationship between Pseudoneutralization assay (logID50) and percent inhibition of ACE2 binding to SARS-CoV-2 spike S1 protein using day 40 pre-challenge sera samples. Assays represent B.1.351 spike protein and B.1.351 pseudovirus. Correlation analyses were performed using the Spearman method.

FIGS. 9A-9D illustrate the study design and durability of humoral immune responses in rhesus macaques primed with INO-4800. FIG. 9A provides a schematic depicting the prime immunization schedule and sample collection timepoints. Note: The longitudinal collection for the NHPs in the 1 mg dose group ended at Week 35 and for 2 mg dose group at Week 52. FIG. 9B shows longitudinal serum IgG binding titers in rhesus macaques vaccinated with 1 or 2 mg INO-4800 at weeks 0 and 4. Antibody titers in the sera were measured against the wildtype SARS-CoV-2 Spike protein antigen. Antibody titers in the sera were also measured against the SARS-CoV-2 S1, SARS-CoV-2 S2 and RBD proteins (FIG. 9D). FIG. 9C shows longitudinal pseudovirus neutralizing activity (ID₅₀) in NHPs primed with INO-4800, measured against SARS-CoV-2 pseudotyped viral stocks for the ancestral (wild-type; Wuhan-Hu-1) SARS-CoV-2 as well as Alpha (B.1.1.7), Beta (B.1.351), and Gamma (P.1) pseudoviruses.

FIGS. 10A-10C illustrate humoral immune responses following homologous or heterologous boost in INO-4800-primed rhesus macaques. Antibody responses were measured in animals boosted with 1 mg of either the homologous INO-4800 (purple symbols) or heterologous INO-4802 (blue symbols) vaccines on the day of the boost (week 0) and at weeks 2 and 4 post-boost. Red lines and blue lines represent geometric mean titers (GMT) or geometric mean inhibition (GMI) for groups 1 and 2, respectively. FIG. 10A provides a schematic of the boost schedule showing the vaccine groups with the respective animal IDs. FIG. 10B shows serum IgG binding titers in rhesus macaques boosted with INO-4800 or INO-4802. Binding titers were measured against the ancestral, Beta, Delta, Gamma, and Omicron Spike proteins. FIG. 10C illustrates serum pseudovirus neutralizing activity in rhesus macaques boosted with INO-4800 or INO-4802. Neutralizing activity was measured against the ancestral, Beta, Delta, Gamma, and Omicron pseudoviruses. FIG. 10D shows ACE2 blocking activity in the serum collected from rhesus macaques boosted with INO-4800 or INO-4802. Inhibition of ACE2 binding was measured against the ancestral, Beta, Delta, and Gamma Spike proteins. In FIGS. 10B-10D, comparisons between INO-4800- and INO-4802-boosted animals at Weeks 2 and 4 were performed using a Mann Whitney test.

FIGS. 11A and 11B illustrate functional antibody responses following homologous or heterologous boost in INO-4800-primed rhesus macaques. FIG. 11A shows Spearman correlation of ACE2 blocking activity and neutralizing activity among animals boosted with either INO-4800 or INO-4802. Correlations relating to functional antibody responses against the wildtype (left) Beta SARS-CoV-2 (center), and Delta (right) variants at weeks 2 and 4 post-boost are shown. FIG. 11B shows Spearman correlation of the frequency of circulating T follicular helper cells with ACE-2 binding inhibition at week 2 post-boost.

FIG. 12 illustrates the efficacy of INO-4802 protection against WT, B.1.1.7, and P.1 VOCs. As of the time of testing, against all SARS-CoV-2 VOCs tested, maintenance of body weight of INO-4802 vaccinated animals compared to controls is observed.

FIGS. 13A and 13B show human ACE2 blocking of B.1.617.2 spike binding by serum from vaccinated hamsters and weight change in hamsters after challenge with B.1.617.2. For FIG. 13A, Syrian Golden Hamsters received IM+EP immunizations with 10 μg pWT, p.B1.351 or INO-4802 on days 0 and 14. Sera collected on day 22 were tested for capacity to block binding of human ACE-2 to B.1.617.2-spike in an electrochemiluminescent-based ELISA assay (mean % inhibition+/−SEM). Not significant (ns) determined by Welch's t test. For FIG. 13B, animals received ID+EP immunizations with 100 μg INO-4802 on days 0 and 21. Hamsters were challenged on day 70 IN with B.1.617.2 and observed for weight loss. Weight change of INO-4802 vaccinated hamsters compared to unvaccinated animals following challenge with B.1.617.2 (Mann-Whitney-test).

FIGS. 14A-14L illustrate cellular immune responses following homologous or heterologous boost in INO-4800-primed rhesus macaques. T cell responses were measured in animals boosted with 1 mg of either the homologous INO-4800 (FIGS. 14A-14F) or heterologous INO-4802 (FIGS. 14G-14L) vaccines on the day of the boost (week 0) and at week 2 post-boost. FIGS. 14A-14C) CD4 and FIGS. 14D-14F) CD8T cell responses in INO-4800-boosted animals against ancestral or Beta derived peptide pools. FIGS. 14G-11I show CD4 and FIGS. 14J-14L show CD8 T cell responses in INO-4802-boosted animals against ancestral or Beta derived peptide pools. The sum of IFNγ, IL-2, and TNF responses are represented in FIGS. 14C, 14F, 14I, and 14L. Bars represent median.

### DETAILED DESCRIPTION OF ILLUSTRATIVE EMBODIMENTS

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “comprising” is intended to include examples encompassed by the terms “consisting essentially of” and “consisting of”; similarly, the term “consisting essentially of” is intended to include examples encompassed by the term “consisting of.” The present disclosure also contemplates other embodiments “comprising,” “consisting of” and “consisting essentially of” the embodiments or elements presented herein, whether explicitly set forth or not.

It is to be appreciated that certain features of the disclosed materials and methods which are, for clarity, described herein in the context of separate embodiments, may also be provided in combination in a single embodiment. Conversely, various features of the disclosed materials and methods that are, for brevity, described in the context of a single embodiment, may also be provided separately or in any subcombination.

The singular forms “a,” “and” and “the” include plural references unless the context clearly dictates otherwise.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value. When values are expressed as approximations by use of the antecedent “about,” it will be understood that the particular value forms another embodiment. Reference to a particular numerical value includes at least that particular value unless the context clearly dictates otherwise.

“Adjuvant” as used herein means any molecule added to an immunogenic composition or vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′) 2, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

The term “biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extent the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below.

“Fragment” or “immunogenic fragment” with respect to polypeptide sequences means a polypeptide capable of eliciting an immune response in a mammal that cross reacts with a reference full-length SARS-CoV-2 antigen. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases in which the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

The INO-4800 drug product contains 10 mg/mL of the DNA plasmid pGX9501 (or INO-4800) in 1× SSC buffer (150 mM sodium chloride and 15 mM sodium citrate).

The INO-4802 drug product contains 10 mg/mL of the DNA plasmid pGX9527 (or INO-4802) in 1× SSC buffer (150 mM sodium chloride and 15 mM sodium citrate).

“Nucleic acid” or “oligonucleotide” or “polynucleotide” or “nucleic acid molecule” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double-stranded or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, and CMV IE promoter.

“Signal peptide” and “leader sequence” are used interchangeably herein and refer to an amino acid sequence that can be linked at the amino terminus of a SARS-CoV-2 protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants or is in need of being immunized with a herein described immunogenic composition or vaccine. The mammal can be a human, chimpanzee, guinea pig, dog, cat, horse, cow, mouse, hamster, rabbit, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting an animal from a disease through means of preventing, suppressing, repressing, or completely eliminating the disease. Preventing the disease involves administering an immunogenic composition or a vaccine of the present invention to an animal prior to onset of the disease. Suppressing the disease involves administering an immunogenic composition or a vaccine of the present invention to an animal after induction of the disease but before its clinical appearance. Repressing the disease involves administering an immunogenic composition or a vaccine of the present invention to an animal after clinical appearance of the disease.

As used herein, unless otherwise noted, the term “clinically proven” (used independently or to modify the terms “safe” and/or “effective”) shall mean that it has been proven by a clinical trial wherein the clinical trial has met the approval standards of U.S. Food and Drug Administration, EMA or a corresponding national regulatory agency. For example, proof may be provided by the clinical trial(s) described in the examples provided herein.

The term “clinically proven safe”, as it relates to a dose, dosage regimen, treatment or method with a SARS-CoV-2 antigen (for example, a SARS-CoV-2 spike antigen administered as pGX9527 or INO-4802 drug product or a biosimilar thereof) refers to a favorable risk:benefit ratio with an acceptable frequency and/or acceptable severity of treatment-emergent adverse events (referred to as AEs or TEAEs) compared to the standard of care or to another comparator. An adverse event is an untoward medical occurrence in a patient administered a medicinal product. One index of safety is the National Cancer Institute (NCI) incidence of adverse events (AE) graded per Common Toxicity Criteria for Adverse Events CTCAE v4.03.

The terms “clinically proven efficacy” and “clinically proven effective” as used herein in the context of a dose, dosage regimen, treatment or method refer to the effectiveness of a particular dose, dosage or treatment regimen. Efficacy can be measured based on change in the course of the disease in response to an agent of the present invention. For example, a SARS-CoV-2 antigen (for example, a SARS-CoV-2 spike antigen administered as pGX9527 or INO-4802 drug product or a biosimilar thereof) is administered to a patient in an amount and for a time sufficient to induce an improvement, preferably a sustained improvement, in at least one indicator that reflects the severity of the disorder that is being treated. Various indicators that reflect the extent of the subject's illness, disease or condition may be assessed for determining whether the amount and time of the treatment is sufficient. Such indicators include, for example, clinically recognized indicators of disease severity, symptoms, or manifestations of the disorder in question. The degree of improvement generally is determined by a physician, who may make this determination based on signs, symptoms, biopsies, or other test results, and who may also employ questionnaires that are administered to the subject, such as quality-of-life questionnaires developed for a given disease. For example, a SARS-CoV-2 antigen (for example, a SARS-CoV-2 spike antigen administered as pGX9527 or INO-4802 drug product or a biosimilar thereof) may be administered to achieve an improvement in a patient's condition related to SARS-CoV-2 infection. Improvement may be indicated by an improvement in an index of disease activity, by amelioration of clinical symptoms or by any other measure of disease activity.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Nucleic Acid Molecules, Antigens, and Immunogenic Compositions

Provided herein are immunogenic compositions, such as vaccines, comprising a nucleic acid molecule encoding a SARS-CoV-2 spike antigen, a fragment thereof, a variant thereof, or a combination thereof. Also provided herein are immunogenic compositions, such as vaccines, comprising a SARS-CoV-2 spike antigen, a fragment thereof, a variant thereof, or a combination thereof. The immunogenic compositions can be used to protect against and treat any number of strains of SARS-CoV-2, thereby treating, preventing, and/or protecting against SARS-CoV-2-based pathologies. The immunogenic compositions can significantly induce an immune response of a subject administered the immunogenic compositions, thereby protecting against and/or treating SARS-CoV-2 infection.

The immunogenic composition can be a DNA vaccine, a peptide vaccine, or a combination DNA and peptide vaccine. The DNA vaccine can include a nucleic acid molecule encoding the SARS-CoV-2 spike antigen. According to some embodiments, the nucleic acid molecule comprises the nucleic acid sequence of nucleotides 55 to 3831 of SEQ ID NO: 2; the nucleic acid sequence of SEQ ID NO: 2; the nucleic acid sequence of SEQ ID NO: 3; or pGX9527. The nucleic acid molecule can be DNA, RNA, cDNA, a variant thereof, a fragment thereof, or a combination thereof. The nucleic acid molecule can also include additional sequences that encode linker, leader, or tag sequences that are linked to the nucleic acid molecule encoding the SARS-CoV-2 spike antigen by a peptide bond. The peptide vaccine can include a SARS-CoV-2 antigenic peptide, a SARS-CoV-2 antigenic protein (optionally a SARS-CoV-2 spike antigen comprising the amino acid sequence of residues 19 to 1277 of SEQ ID NO: 1 or the amino acid sequence of SEQ ID NO: 1), a variant thereof, a fragment thereof, or a combination thereof. The combination DNA and peptide vaccine can include the above described nucleic acid molecule encoding the SARS-CoV-2 spike antigen and the SARS-CoV-2 spike antigenic peptide or protein, in which the SARS-CoV-2 spike antigenic peptide or protein and the encoded SARS-CoV-2 spike antigen have the same or different amino acid sequence.

The disclosed immunogenic compositions can elicit both humoral and cellular immune responses that target the SARS-CoV-2 spike antigen in the subject administered the immunogenic composition. The disclosed immunogenic compositions can elicit neutralizing antibodies and immunoglobulin G (IgG) antibodies that are reactive with the SARS-CoV-2 spike antigen. The immunogenic compositions can also elicit CD8+ and CD4+ T cell responses that are reactive to the SARS-CoV-2 spike antigen and produce interferon-gamma (IFN-γ), interleukin-2 (IL-2), TNFα, interleukin-4 (IL-4), circulating T follicular helper (Tfh) cells, or any combination thereof.

The immunogenic compositions can induce a humoral immune response in the subject administered the immunogenic composition. The induced humoral immune response can be specific for the SARS-CoV-2 spike antigen. The induced humoral immune response can be reactive with the SARS-CoV-2 spike antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the immunogenic compositions can include an increased level of neutralizing antibodies associated with the subject administered the immunogenic composition as compared to a subject not administered the immunogenic composition or to a subject administered INO-4800. The neutralizing antibodies can be specific for the SARS-CoV-2 spike antigen. The neutralizing antibodies can be reactive with the SARS-CoV-2 spike antigen. The neutralizing antibodies can provide protection against and/or treatment of SARS-CoV-2 infection and its associated pathologies in the subject administered the immunogenic composition.

The humoral immune response induced by the immunogenic compositions can include an increased level of IgG antibodies associated with the subject administered the immunogenic composition as compared to a subject not administered the immunogenic composition or to a subject administered INO-4800. These IgG antibodies can be specific for the SARS-CoV-2 spike antigen. These IgG antibodies can be reactive with the SARS-CoV-2 spike antigen. The level of IgG antibody associated with the subject administered the immunogenic composition can be increased by about 1.5-fold to about 16-fold, about 2-fold to about 12-fold, or about 3-fold to about 10-fold as compared to the subject not administered the immunogenic composition or to a subject administered INO-4800. The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the immunogenic composition or to a subject administered INO-4800.

The immunogenic compositions can induce a cellular immune response in the subject administered the immunogenic composition. The induced cellular immune response can be specific for the SARS-CoV-2 spike antigen. The induced cellular immune response can be reactive to the SARS-CoV-2 spike antigen. The induced cellular immune response can include eliciting a CD8+ T cell response. The elicited CD8+ T cell response can be reactive with the SARS-CoV-2 spike antigen. The elicited CD8+ T cell response can be polyfunctional. The induced cellular immune response can include eliciting a CD8+ T cell response, in which the CD8+ T cells produce interferon-gamma (IFN-γ), interleukin-2, and/or upregulation of CD107a.

The induced cellular immune response can include an increased CD8+ T cell response associated with the subject administered the immunogenic composition as compared to the subject not administered the immunogenic composition or to a subject administered INO-4800. The CD8+ T cell response associated with the subject administered the immunogenic composition can be increased by about 2-fold to about 30-fold, about 3-fold to about 25-fold, or about 4-fold to about 20-fold as compared to the subject not administered the immunogenic composition or to a subject administered INO-4800. The CD8+ T cell response associated with the subject administered the immunogenic composition can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the immunogenic composition or to a subject administered INO-4800.

The cellular immune response induced by the immunogenic composition can include eliciting a CD4+ T cell response. The elicited CD4+ T cell response can be reactive with the SARS-CoV-2 antigen. The elicited CD4+ T cell response can be polyfunctional. The induced cellular immune response can include eliciting a CD4+ T cell response, in which the CD4+ T cells produce IFN-γ, interleukin-2 (IL-2), interleukin-4 (IL-4), Tumour Necrosis Factor alpha (TNFα), or any combination thereof.

The cellular immune response induced by the immunogenic composition can include an increase in circulating Tfh (CXCR5+ PD-1+) cells.

The immunogenic composition of the present invention can have features required of effective immunogenic compositions such as being safe so the immunogenic composition itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The immunogenic composition can further induce an immune response when administered to different tissues such as the muscle or skin. The immunogenic composition can further induce an immune response when parenterally administered, for example by subcutaneous, intradermal, or intramuscular injection, optionally followed by electroporation as described herein.

### a. SARS-CoV-2 Antigen and Nucleic Acid Molecules Encoding the Same

As described above, provided herein are immunogenic compositions comprising a nucleic acid molecule encoding a SARS-CoV-2 spike antigen, a fragment thereof, a variant thereof, or a combination thereof. Also provided herein are immunogenic compositions comprising a SARS-CoV-2 spike antigen, a fragment thereof, a variant thereof, or a combination thereof.

The SARS-CoV-2 spike antigen is capable of eliciting an immune response in a mammal against one or more SARS-CoV-2 strains. The SARS-CoV-2 spike antigen can comprise an epitope(s) that makes it particularly effective as an immunogen against which an anti- SARS-CoV-2 immune response can be induced.

The SARS-CoV-2 antigen can be a consensus antigen derived from two or more strains of SARS-CoV-2. In some embodiments, the SARS-CoV-2 antigen is a SARS-CoV-2 consensus spike antigen. The SARS-CoV-2 consensus spike antigen can be derived from the sequences of spike antigens from multiple strains of SARS-CoV-2, and thus, the SARS-CoV-2 consensus spike antigen is unique. The immunogenic compositions of the present invention are thus widely applicable to multiple strains of SARS-CoV-2 because of the unique sequences of the SARS-CoV-2 consensus spike antigen. These unique sequences allow the vaccine to be protective against multiple strains of SARS-CoV-2, including genetically diverse variants of SARS-CoV-2.

Nucleic acid molecules encoding the SARS-CoV-2 antigen can be modified for improved expression. Modification can include codon optimization, RNA optimization, addition of a kozak sequence for increased translation initiation, and/or the addition of an immunoglobulin leader sequence to increase the immunogenicity of the SARS-CoV-2 spike antigen. The SARS-CoV-2 spike antigen can comprise a signal peptide such as an immunoglobulin signal peptide, for example, but not limited to, an immunoglobulin E (IgE) or immunoglobulin (IgG) signal peptide. In some embodiments, the SARS-CoV-2 spike antigen can comprise a hemagglutinin (HA) tag. The SARS-CoV-2 spike antigen can be designed to elicit stronger and broader cellular and/or humoral immune responses than a corresponding codon optimized spike antigen.

In some embodiments, the SARS-CoV-2 spike antigen comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of residues 19 to 1277 of SEQ ID NO: 1. In some embodiments the SARS-CoV-2 spike antigen comprises the amino acid sequence set forth in residues 19 to 1277 of SEQ ID NO: 1. In some embodiments, the SARS-CoV-2 spike antigen comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of SEQ ID NO: 1. In some embodiments the SARS-CoV-2 spike antigen comprises the amino acid sequence of SEQ ID NO: 1.

In some embodiments the nucleic acid molecule encoding the SARS-CoV-2 spike antigen comprises the nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity to the sequence set forth in nucleotides 55 to 3831 of SEQ ID NO: 2, SEQ ID NO: 2, or SEQ ID NO: 3.

In some embodiments the SARS-CoV-2 spike antigen is operably linked to an IgE leader sequence. In some such embodiments, the SARS-CoV-2 spike antigen comprises the amino acid sequence set forth in SEQ ID NO: 1. In some embodiments, the SARS-CoV-2 spike antigen having an IgE leader sequence is encoded by the nucleotide sequence set forth in SEQ ID NO:2 or SEQ ID NO: 3.

Immunogenic fragments of SEQ ID NO:1 are provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:1. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:1 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:1. Some embodiments relate to immunogenic fragments that have 96% homology to the immunogenic fragments of consensus protein sequences herein. Some embodiments relate to immunogenic fragments that have 97% homology to the immunogenic fragments of consensus protein sequences herein. Some embodiments relate to immunogenic fragments that have 98% homology to the immunogenic fragments of consensus protein sequences herein. Some embodiments relate to immunogenic fragments that have 99% homology to the immunogenic fragments of consensus protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:1. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:1. Immunogenic fragments can be at least 95%, at least 96%, at least 97% at least 98% or at least 99% homologous to fragments of SEQ ID NO:1. In some embodiments, immunogenic fragments include sequences that encode a leader sequence, such as for example an immunoglobulin leader, such as the IgE leader. In some embodiments, fragments are free of coding sequences that encode a leader sequence.

### b. Vector

The immunogenic compositions can comprise one or more vectors that include a nucleic acid molecule encoding the SARS-CoV-2 spike antigen. The one or more vectors can be capable of expressing the spike antigen. The vector can have a nucleic acid sequence containing an origin of replication. The vector can be a plasmid, bacteriophage, bacterial artificial chromosome or yeast artificial chromosome. The vector can be either a self-replicating extrachromosomal vector or a vector which integrates into a host genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

### (1) Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

### (2) Circular and Linear Vectors

The vector may be a circular plasmid, which may transform a target cell by integration into the cellular genome or exist extrachromosomally (e.g., autonomous replicating plasmid with an origin of replication).

The vector can be pVAX, pcDNA3.0, pGX0001, or provax, or any other expression vector capable of expressing DNA encoding the antigen and enabling a cell to translate the sequence to an antigen that is recognized by the immune system.

Also provided herein is a linear nucleic acid immunogenic composition, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

The LEC may be derived from any plasmid capable of being linearized. The plasmid may be capable of expressing the antigen. The plasmid can be pNP (Puerto Rico/34) or pM2 (New Caledonia/99). The plasmid may be WLV009, pVAX, pGX0001, pcDNA3.0, or provax, or any other expression vector capable of expressing DNA encoding the antigen and enabling a cell to translate the sequence to an antigen that is recognized by the immune system.

The LEC can be perM2. The LEC can be perNP. perNP and perMR can be derived from pNP (Puerto Rico/34) and pM2 (New Caledonia/99), respectively.

### (3) Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

### c. Excipients and Other Components of the Immunogenic Compositions

The immunogenic compositions may further comprise a pharmaceutically acceptable excipient. The pharmaceutically acceptable excipient can be functional molecules such as vehicles, carriers, buffers, or diluents. As used herein. “buffer” refers to a buffered solution that resists changes in pH by the action of its acid-base conjugate components. The buffer generally has a pH from about 4.0 to about 8.0, for example from about 5.0 to about 7.0. In some embodiments, the buffer is saline-sodium citrate (SSC) buffer. In some embodiments in which the immunogenic composition comprises a nucleic acid molecule encoding a SARS-CoV-2 spike antigen as described above, the immunogenic composition comprises 10 mg/ml of vector in buffer, for example but not limited to SSC buffer. In some embodiments, the immunogenic composition comprises 10 mg/mL of the DNA plasmid pGX9527 in buffer. In some embodiments, the immunogenic composition is stored at about 2° C. to about 8° C. In some embodiments, the immunogenic composition is stored at room temperature. The immunogenic composition may be stored for at least a year at room temperature. In some embodiments, the immunogenic composition is stable at room temperature for at least a year, wherein stability is defined as a supercoiled plasmid percentage of at least about 80%. In some embodiments, the supercoiled plasmid percentage is at least about 85% following storage for at least a year at room temperature.

The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent may be a polyanion, polycation, including poly-L-glutamate (LGS), or lipid. The transfection facilitating agent is poly-L-glutamate, and the poly-L-glutamate may be present in the immunogenic composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid immunogenic compositions may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the immunogenic composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in an alternative plasmid or are delivered as proteins in combination with the plasmid above in the immunogenic composition. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The immunogenic composition can be formulated according to the mode of administration to be used. According to some embodiments, the immunogenic composition is formulated in a buffer, optionally saline-sodium citrate buffer. For example, the immunogenic composition may formulated at a concentration of 10 mg nucleic acid molecule per milliliter of buffer, optionally a sodium salt citrate buffer. An injectable immunogenic pharmaceutical composition can be sterile, pyrogen free and particulate free. An isotonic formulation or solution can be used. Additives for isotonicity can include sodium chloride, dextrose, mannitol, sorbitol, and lactose. The immunogenic composition can comprise a vasoconstriction agent. The isotonic solutions can include phosphate buffered saline. Immunogenic compositions can further comprise stabilizers including gelatin and albumin. The stabilizers can allow the formulation to be stable at room or ambient temperature for extended periods of time, including LGS or polycations or polyanions.

Also provided herein are articles of manufacture comprising the immunogenic composition. In some embodiments, the article of manufacture is a container holding the immunogenic composition. The container may be, for example but not limited to, a syringe or a vial. The vial may have a stopper pierceable by a syringe.

The immunogenic composition can be packaged in suitably sterilized containers such as ampules, bottles, or vials, either in multi-dose or in unit dosage forms. The containers are preferably hermetically sealed after being filled with a vaccine preparation. Preferably, the vaccines are packaged in a container having a label affixed thereto, which label identifies the vaccine, and bears a notice in a form prescribed by a government agency such as the United States Food and Drug Administration reflecting approval of the vaccine under appropriate laws, dosage information, and the like. The label preferably contains information about the vaccine that is useful to a health care professional administering the vaccine to a patient. The package also preferably contains printed informational materials relating to the administration of the vaccine, instructions, indications, and any necessary required warnings.

### Methods of Vaccination

Also provided herein are methods of treating, protecting against, and/or preventing disease in a subject in need thereof by administering an immunogenic composition of the invention to the subject. Administration of the immunogenic composition to the subject can induce or elicit an immune response in the subject. The induced immune response can be used to treat, prevent, and/or protect against disease, for example, pathologies relating to SARS-CoV-2 infection. The induced immune response in the subject administered the immunogenic composition can provide resistance to one or more SARS-CoV-2 strains.

The induced immune response can include an induced humoral immune response and/or an induced cellular immune response. The humoral immune response can be induced by about 1.5-fold to about 16-fold, about 2-fold to about 12-fold, or about 3-fold to about 10-fold relative to the subject's baseline or to a subject who is not administered the immunogenic composition or to a subject administered INO-4800. The induced humoral immune response can include IgG antibodies and/or neutralizing antibodies that are reactive to the antigen. The induced cellular immune response can include a CD8+ T cell response, which is induced by about 2-fold to about 30-fold, about 3-fold to about 25-fold, or about 4-fold to about 20-fold. The induced cellular immune response can include a CD4+ T cell response, which is induced by about 2-fold to about 30-fold, about 3-fold to about 25-fold, or about 4-fold to about 20-fold. The induced cellular immune response can include an increase in Tfh cells by about 2-fold to about 30-fold, about 3-fold to about 25-fold, or about 4-fold to about 20-fold.

The vaccine dose can be between 1 μg to 10 mg active component/kg body weight/time and can be 20 μg to 10 mg component/kg body weight/time. The vaccine can be administered every 21, 22, 23, 24, 25, 26, 27, 28, 29, 30, 31, 32, 33, 34, 35, 36, 37, 38, 39, 40 or more days or every 3, 4, 5, 6, 7, 8, 9, 10, 11, 12, 13, 14, 15, 16, 17, 18, 19, 20 or more weeks. The number of vaccine doses for effective treatment can be 1, 2, 3, 4, 5, 6, 7, 8, 9, 10, or more.

### a. Administration

The immunogenic composition can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The vaccine may be administered, for example, in one, two, three, four, or more injections. In some embodiments, an initial dose of about 0.5 mg to about 2.0 mg of the nucleic acid molecule is administered to the subject. The initial dose may be administered in one, two, three, or more injections. The initial dose may be followed by administration of one, two, three, four, or more subsequent doses of about 0.5 mg to about 2.0 mg of the nucleic acid molecule about one, two, three, four, five, six, seven, eight, ten, twelve or more weeks after the immediately prior dose. Each subsequent dose may be administered in one, two, three, or more injections. In some embodiments, the immunogenic composition is administered to the subject before, with, or after an additional agent. In some embodiments, the immunogenic composition is administered as a booster following administration of an agent for the treatment of SARS-CoV-2 infection or the treatment or prevention of a disease or disorder associated with SARS-CoV-2 infection. The agent for the treatment of SARS-CoV-2 infection or the treatment or prevention of a disease or disorder associated with SARS-CoV-2 infection may be, for example but not limited to, a SARS-CoV-2 wild-type matched vaccine, pGX9501, INO-4800 drug product or a biosimilar thereof. In one embodiment, the disease or disorder associated with SARS-CoV-2 infection includes, but is not limited to, to Coronavirus Disease 2019 (COVID-19). In some embodiments, the disease or disorder associated with SARS-CoV-2 infection is Multisystem inflammatory syndrome in adults (MIS-A) or Multisystem inflammatory syndrome in children (MIS-C).

The subject can be a mammal, such as a human, a horse, a nonhuman primate, a cow, a pig, a sheep, a cat, a dog, a guinea pig, a rabbit, a rat, a mouse, or a hamster.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety). Parenteral administration may optionally be followed with electroporation as described herein.

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system are described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15-volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA® (Inovio Pharmaceuticals, Blue Bell Pa.) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra® device and system is described in U.S. Pat. No. 7,245,963, the contents of which are herein incorporated by reference. The CELLECTRA® device may be the CELLECTRA 2000® device or CELLECTRA® 3PSP device. The CELLECTRA® 2000 device is configured by the manufacturer to support either ID (intradermal) or IM (intramuscular) administration. The CELLECTRA™ 2000 includes the CELLECTRA™ Pulse Generator, the appropriate applicator, disposable sterile array and disposable sheath (ID only). The DNA plasmid is delivered separately via needle and syringe injection in the area delineated by the electrodes immediately prior to the electroporation treatment.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

### Use in Combination

In some embodiments, the present invention provides a method of treating, protecting against, and/or preventing a SARS-CoV-2 infection, or treating, protecting against, and/or preventing a disease or disorder associated with SARS-CoV-2 infection, in a subject in need thereof by administering a combination of a nucleic acid molecule encoding a SARS-CoV-2 spike antigen as disclosed herein, or fragment or variant thereof, in combination with one or more additional agents for treating, protecting against, and/or preventing of SARS-CoV-2 infection or the treatment or prevention of disease or disorder associated with SARS-CoV-2 infection. In some embodiments, the disease or disorder associated with SARS-CoV-2 infection is Coronavirus Disease 2019 (COVID-19), Multisystem inflammatory syndrome in adults (MIS-A), or Multisystem inflammatory syndrome in children (MIS-C).

The nucleic acid molecule encoding a SARS-CoV-2 spike antigen and additional agent may be administered using any suitable method such that a combination of the nucleic acid molecule encoding a SARS-CoV-2 spike antigen and the additional agent are both present in the subject. In one embodiment, the method may comprise administration of a first composition comprising an agent for the prevention or treatment of SARS-CoV-2 infection or the treatment or prevention of disease or disorder associated with SARS-CoV-2 infection and administration of a second composition comprising a nucleic acid molecule encoding a SARS-CoV-2 spike antigen as disclosed herein less than 1, less than 2, less than 3, less than 4, less than 5, less than 6, less than 7, less than 8, less than 9 or less than 10 days following administration of the first composition comprising the agent for the treatment or prevention of SARS-CoV-2 infection or the treatment or prevention of disease or disorder associated with SARS-CoV-2 infection. In one embodiment, the method may comprise administration of a first composition comprising a nucleic acid molecule encoding a SARS-CoV-2 spike antigen as disclosed herein and administration of a second composition comprising an agent for the treatment or prevention of SARS-CoV-2 infection or the treatment or prevention of disease or disorder associated with SARS-CoV-2 infection less than 1, less than 2, less than 3, less than 4, less than 5, less than 6, less than 7, less than 8, less than 9 or less than 10 days following administration of the nucleic acid molecule encoding a SARS-CoV-2 spike antigen. In one embodiment, the method may comprise administration of a first composition comprising an agent for the treatment or prevention of SARS-CoV-2 infection or the treatment or prevention of disease or disorder associated with SARS-CoV-2 infection and a second composition comprising a nucleic acid molecule encoding a SARS-CoV-2 spike antigen as disclosed herein concurrently. In one embodiment, the method may comprise administration of a single composition comprising an agent for the treatment or prevention of SARS-CoV-2 infection or the treatment or prevention of disease or disorder associated with SARS-CoV-2 infection and a nucleic acid molecule encoding a SARS-CoV-2 spike antigen as disclosed herein, optionally a SARS-CoV-2 spike antigen comprising the amino acid sequence of residues 19 to 1277 of SEQ ID NO: 1 or the amino acid sequence of SEQ ID NO: 1. In accordance with some embodiments of the disclosed methods, the nucleic acid molecule encoding a SARS-CoV-2 spike antigen comprises the nucleic acid sequence of nucleotides 55 to 3831 of SEQ ID NO: 2; the nucleic acid sequence of SEQ ID NO: 2; the nucleic acid sequence of SEQ ID NO: 3; pGX9527, INO-4802 drug product, or a biosimilar thereof.

In some embodiments, the agent for the treatment or prevention of SARS-CoV-2 infection or the treatment or prevention of disease or disorder associated with SARS-CoV-2 infection is a therapeutic agent. In one embodiment, the therapeutic agent is an antiviral agent. In one embodiment, the therapeutic agent is an antibiotic agent.

Non-limiting examples of antibiotics that can be used in combination with the a nucleic acid molecule encoding a SARS-CoV-2 antigen of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

### Administration as a Booster

In one embodiment, the immunogenic composition is administered as a booster vaccine following administration of an initial agent or vaccine for the treatment or prevention of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C). In accordance with the disclosed methods, the initial agent for the treatment or prevention of SARS-CoV-2 infection or the treatment or prevention of disease or disorder associated with SARS-CoV-2 infection may be, for example but not limited to, a SARS-CoV-2 wild-type matched vaccine, pGX9501, INO-4800 or a biosimilar thereof. In some embodiments, the booster vaccine comprises a nucleic acid molecule encoding a SARS-CoV-2 spike antigen, optionally a SARS-CoV-2 spike antigen comprising the amino acid sequence of residues 19 to 1277 of SEQ ID NO: 1 or the amino acid sequence of SEQ ID NO: 1. According to some embodiments, the nucleic acid molecule comprises the nucleic acid sequence of nucleotides 55 to 3831 of SEQ ID NO: 2; the nucleic acid sequence of SEQ ID NO: 2; the nucleic acid sequence of SEQ ID NO: 3; pGX9527, INO-4802 drug product, or a biosimilar thereof.

In some embodiments, the booster vaccine is administered at least once, at least twice, at least 3 times, at least 4 times, or at least 5 times following administration of an initial agent or vaccine for the treatment or prevention of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C). In one embodiment, the booster vaccine is administered at least 8 hours, at least 12 hours, at least 16 hours, at least 20 hours, at least 24 hours, at least 36 hours, at least 48 hours, at least 60 hours, at least 72 hours, at least 4 days, at least 5 days, at least 6 days, at least 1 week at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 1 year or greater than 1 year following administration of an initial agent or vaccine for the treatment or prevention of SARS-CoV-2 infection or the treatment or prevention of a disease or disorder associated with SARS-CoV-2 infection, including, but not limited to COVID-19, Multisystem inflammatory syndrome in adults (MIS-A), or Multisystem inflammatory syndrome in children (MIS-C).

### Use in Assays

In some embodiments, the nucleic acid molecules, or encoded antigens, of the invention can be used in assays in vivo or in vitro. In some embodiments, the nucleic acid molecules, or encoded antigens can be used in assays for detecting the presence of anti-SARS-CoV-2 spike antibodies. Exemplary assays in which the nucleic acid molecules or encoded antigens can be incorporated into include, but are not limited to, Western blot, dot blot, surface plasmon resonance methods, Flow Cytometry methods, various immunoassays, for example, immunohistochemistry assays, immunocytochemistry assays, ELISA, capture ELISA, enzyme-linked immunospot (ELISpot) assays, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, New York; Harlow et al., 1999, Using Antibodies: A Laboratory Manual, Cold Spring Harbor Laboratory Press, NY.

In one embodiment, the SARS-CoV-2 spike antigen, or fragments thereof, of the invention can be used in an assay for intracellular cytokine staining combined with flow cytometry, to assess T-cell immune responses. This assay enables the simultaneous assessment of multiple phenotypic, differentiation and functional parameters pertaining to responding T-cells, most notably, the expression of multiple effector cytokines. These attributes make the technique particularly suitable for the assessment of T-cell immune responses induced by the vaccine of the invention.

In one embodiment, the SARS-CoV-2 spike antigen, or fragments thereof, of the invention can be used in an ELIspot assay. The ELISpot assay is a highly sensitive immunoassay that measures the frequency of cytokine-secreting cells at the single-cell level. In this assay, cells are cultured on a surface coated with a specific capture antibody in the presence or absence of stimuli. In one embodiment, the SARS-CoV-2 spike antigen, or fragments thereof, of the invention can be used as the stimulus in the ELISpot assay.

### Diagnostic Methods

In some embodiments, the invention relates to methods of diagnosing a subject as having SARS-CoV-2 infection or having SARS-CoV-2 antibodies. In some embodiments, the methods include contacting a sample from a subject with a SARS-CoV-2 spike antigen of the invention, or a cell comprising a nucleic acid molecule for expression of the SARS-CoV-2 spike antigen, and detecting binding of an anti-SARS-CoV-2 spike antibody to the SARS-CoV-2 spike antigen of the invention. According to some embodiments, the antigen is encoded by a nucleic acid molecule comprising the nucleic acid sequence of nucleotides 55 to 3831 of SEQ ID NO: 2; the nucleic acid sequence of SEQ ID NO: 2; the nucleic acid sequence of SEQ ID NO: 3; or pGX9527. In some embodiments, the antigen comprises the amino acid sequence of residues 19 to 1277 of SEQ ID NO: 1 or the amino acid sequence of SEQ ID NO: 1. In such an embodiment, binding of an anti-SARS-CoV-2 spike antibody present in the sample of the subject to the antigen, or fragment thereof, of the invention would indicate that the subject is currently infected or was previously infected with SARS-CoV-2.

### Kits and Articles of Manufacture

Provided herein is a kit, which can be used for treating a subject using the method of vaccination described above. The kit can comprise the immunogenic compositions described herein. According to some embodiments, the kit comprises a nucleic acid molecule comprising the nucleic acid sequence of nucleotides 55 to 3831 of SEQ ID NO: 2; the nucleic acid sequence of SEQ ID NO: 2; the nucleic acid sequence of SEQ ID NO: 3; pGX9527, INO-4802 drug product, or a biosimilar thereof.

The kit can also comprise instructions for carrying out the vaccination method described above and/or how to use the kit. Instructions included in the kit can be affixed to packaging material or can be included as a package insert. While instructions are typically written or printed materials, they are not limited to such. Any medium capable of storing instructions and communicating them to an end user is contemplated by this disclosure. Such media include, but are not limited to, electronic storage media (e.g., magnetic discs, tapes, cartridges), optical media (e.g., CD ROM), and the like. As used herein, the term “instructions” can include the address of an internet site which provides instructions.

Further provided herein are articles of manufacture containing the immunogenic composition described herein. In some embodiments, the article of manufacture is a container, such as a vial, optionally a single-use vial. In one embodiment, the article of manufacture is a single-use glass vial equipped with a stopper, which contains the immunogenic composition described herein to be administered. In some embodiments, the vial comprises a stopper, pierceable by a syringe, and a seal. In some embodiments, the article of manufacture is a syringe.

### EXAMPLES

The present invention has multiple aspects, illustrated by the following non-limiting examples.

### Example 1

### Materials and Methods

### pGX9527 and Other Plasmid Constructs

Data on SARS-CoV-2 genome sequence entries (derived from GISAID) covering a four-month period (October 2020-January 2021) were collected from multiple geographic regions (Brazil, Canada, India, Italy, Japan, Nigeria, South Africa, United Kingdom, United States). Mutations in the SARS-CoV-2 Spike sequences were aggregated for each region. Sequence analyses were performed using custom Python scripts (Python Software Foundation, available at https_www_python_org). Molecular modeling of the spike was performed with multiple SARS-CoV-2 spike templates (PDB ID:6XM3, 6VXX, 7K4N) using Prime [Jacobson 2004] from the Bioluminate suite (BioLuminate Release 2018-3, Schrödinger, LLC, New York, N.Y.). Visualization was performed using Bioluminate and Discovery Studio (Discovery Studio 2019, BIOVIA, Dassault Systèmes, San Diego, Calif.).

Data on mutations in the SARS-CoV-2 Spike sequence was aggregated for each region, generating counts of observed regional mutations. The results from the regions were then aggregated to determine a common consensus set of emerging variations in SARS-CoV-2 Spike protein sequences internationally.

Three amino acid changes were placed in the receptor binding domain (RBD) of the SynCon® SARS-CoV-2 Spike to reflect those observed in the SARS-CoV-2 lineage B.1.351 as well as in other emerging variants worldwide. The amino acid changes (K417N/E484K/N501Y) were defined using a reference of the B.1.351 lineage from Tegally H et al. medRxiv 2020. doi.org/10.1101/2020 Dec. 21 20248640) and the PANGO Lineages international lineage reports (Rambaut A et al. Nat Microbiol. 2020. doi: 10.1038/s41564-020-0770-5) as well as data gathered for developing the SynCon® SARS-CoV-2 Spike.

A tandem proline mutation (K986P/V987P) named “2P” was added to the SynCon® SARS-CoV-2 Spike which putatively stabilizes several types of coronavirus spike proteins in a prefusion conformation including that of SARS-CoV-2 (Pallesen J et al. Proc Natl Acad Sci USA. 2017. doi: 10.1073/pnas.1707304114; Kirchdoerfer R N et al. Sci Rep. 2018. doi: 10.1038/s41598-018-34171-7; Xia X. Viruses. 2021 doi: 10.3390/v13010109.).

Aggregation of large numbers of mutations that did not naturally co-occur was avoided to reduce the potential of generating novel non-relevant epitopes. All mutations and changes are numbered according to the canonical SARS-CoV-2 spike sequence numbering scheme.

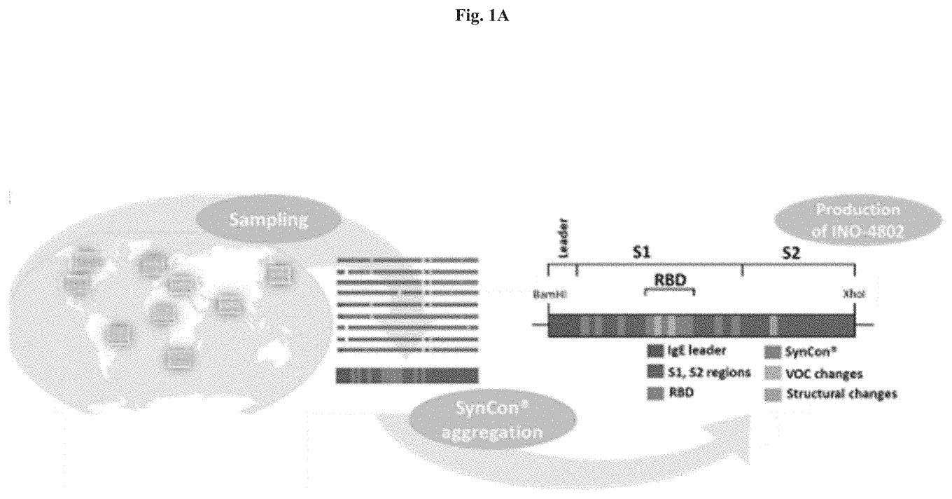

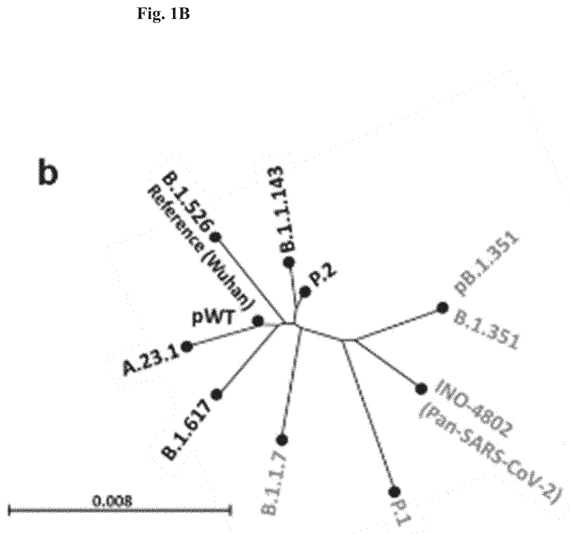

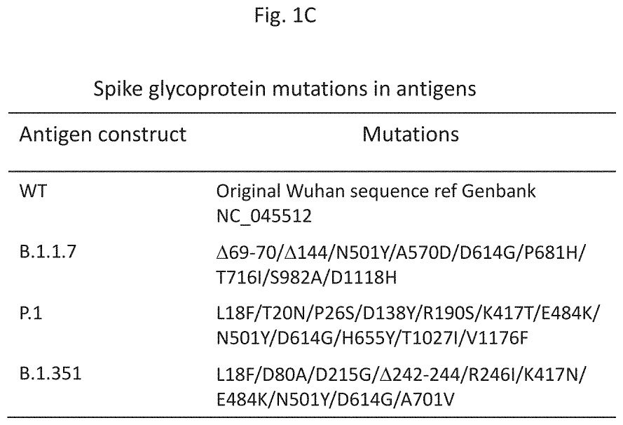

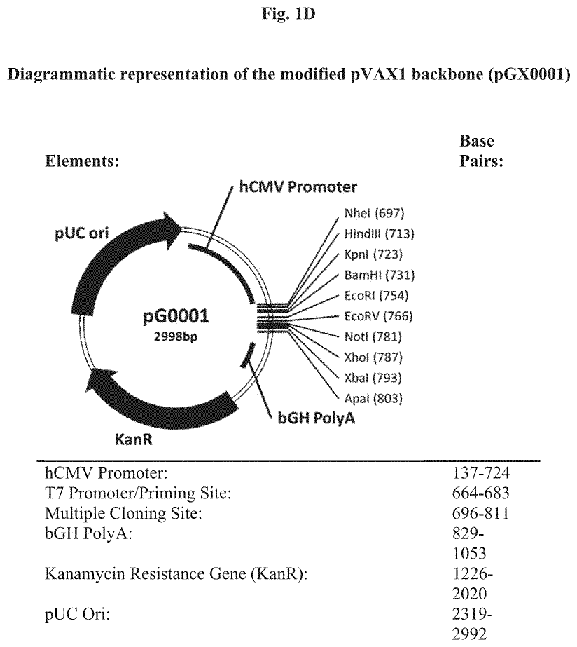

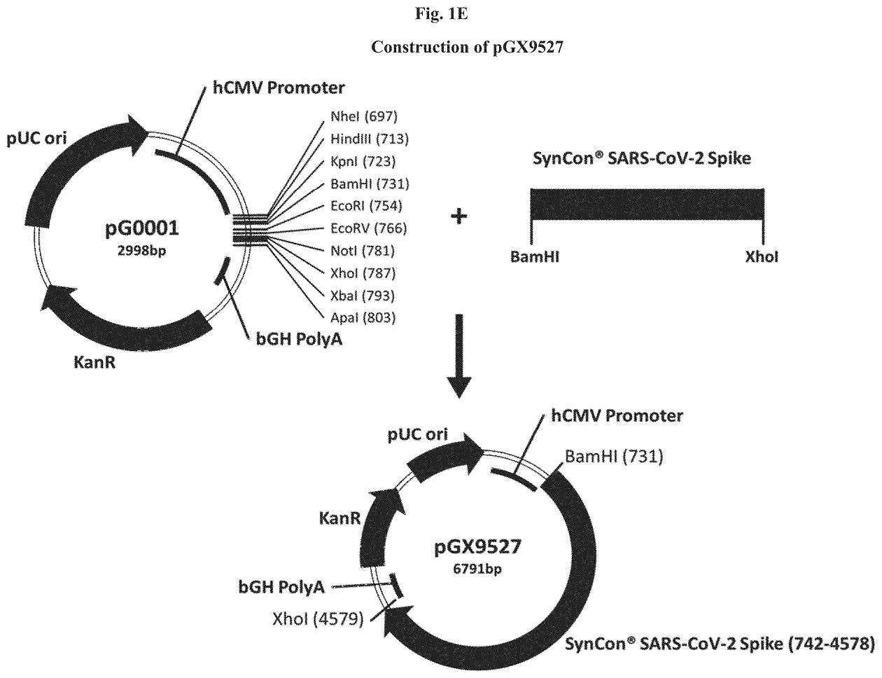

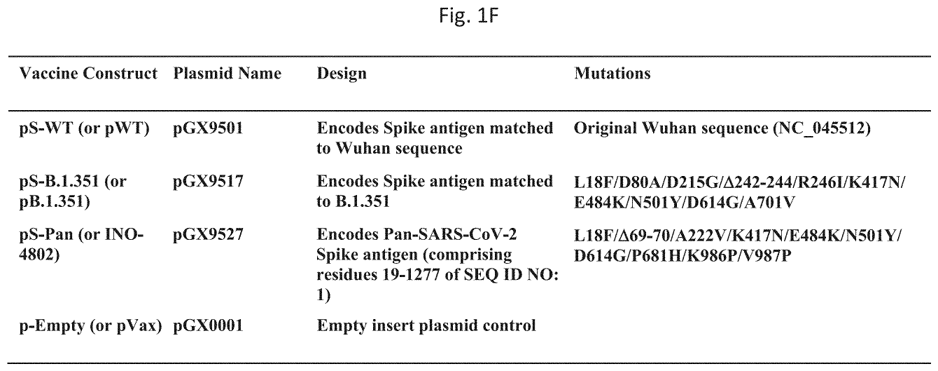

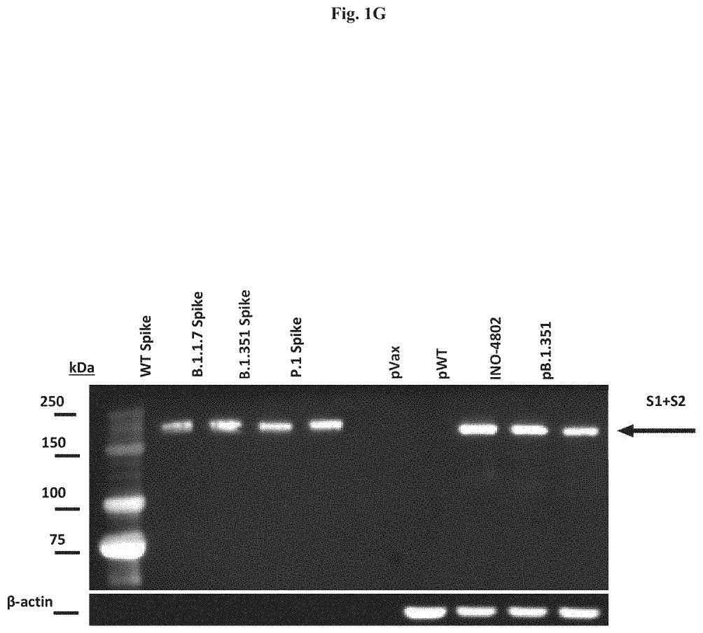

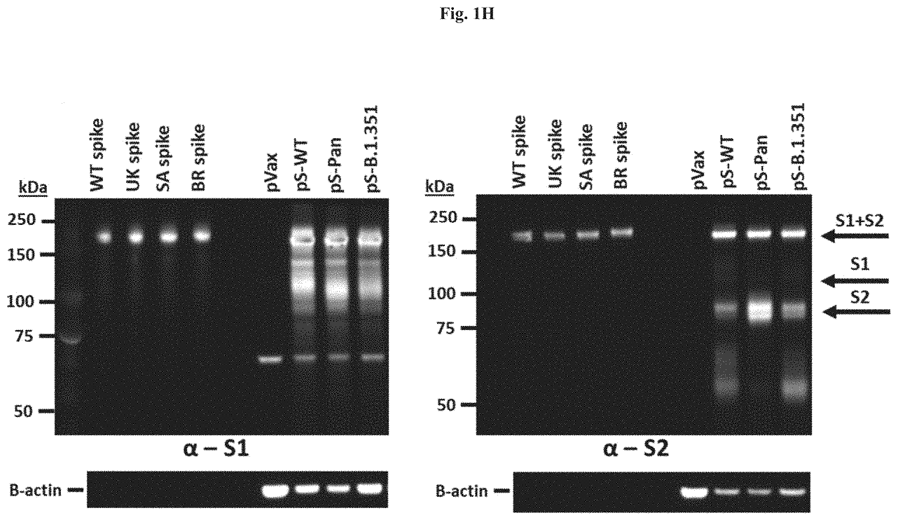

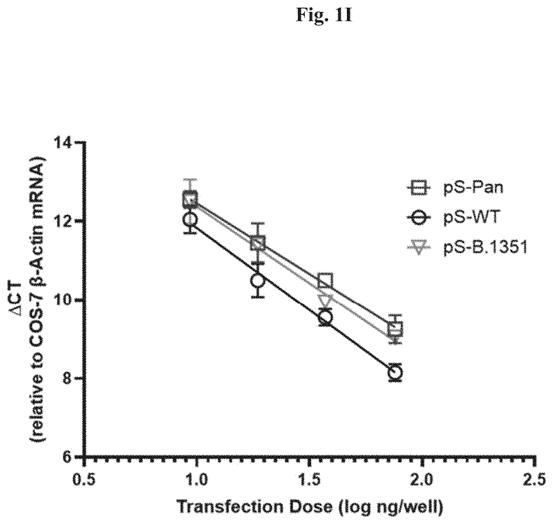

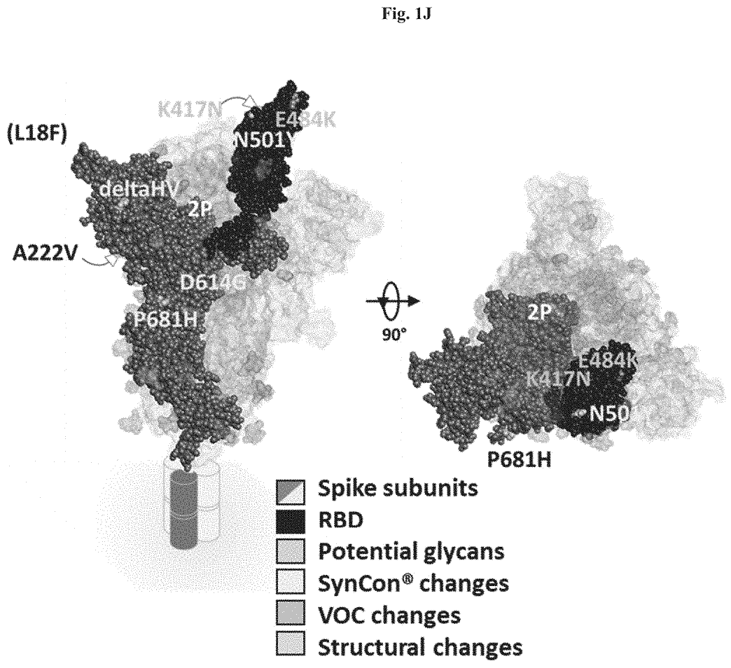

Sequence changes were mapped schematically as well as onto a spike trimer model containing the changes (FIG. 1A and 1J).

The final single construct containing all changes was termed INO-4802. The design strategy results can be visualized using an unrooted phylogenetic tree comparing the spike sequences of INO-4802 to several other constructs used in the studies along with several circulating lineages including multiple VOCs (FIG. 1B). INO-4802 clusters closely but does not overlap with any VOCs. Plasmids matched to WT (pWT) and B.1.351 (pB.1.351) variants were used as control immunogens in this study, they show identity to the matched Spike glycoprotein sequences as expected (FIG. 1F).

An IgE leader sequence also replaced the endogenous SARS-CoV-2 Spike signal peptide sequence.

Included in the construct synthesis was the addition of a Kozak sequence (GCCACC; SEQ ID NO: 14) immediately 5′ of the start codon, in addition to restriction sites for subcloning of the construct into pGX0001 vector (5′ BamHI and 3′XhoI).

Sequence assembly was performed using Geneious Prime® 2020 Feb. 3 (Build 2020 Aug. 25, Biomatters Ltd., Auckland NZ). The optimized DNA sequence was synthesized (Genscript, Piscataway N.J.), digested with BamHI and XhoI, and cloned into the expression vector under the control of the cytomegalovirus immediate-early promoter, generating pGX9527. Strain-matched spike sequences were similarly optimized and cloned into identical restriction site locations into the pGX001 backbone.

Pseudovirus plasmids were designed and constructed as previously described [Andrade V M et al 2021. bioRxiv doi.org/10.1101/2021 Apr. 14 439719].

Dual proline mutations (K986P/V987P, 2P) were added to the SynCon® SARS-CoV-2 Spike antigen. An IgE leader sequence replaced the endogenous SARS-CoV-2 Spike signal peptide sequence. The coding sequence was codon-optimized using Inovio's proprietary optimization algorithm. Included in the construct synthesis was the addition of a Kozak sequence (GCCACC; SEQ ID NO: 14) immediately 5′ of the start codon in addition to restriction sites for subcloning of the construct into pGX0001 vector (5′ BamHI and 3′XhoI). The optimized DNA sequence was synthesized (Genscript, Piscataway, N.J.), digested with BamHI and XhoI, and cloned into the expression vector under the control of the cytomegalovirus immediate-early promoter. pGX9527 (or pS-Pan) is the resulting DNA plasmid expressing the SynCon® SARS-CoV-2 Spike protein (SARS-CoV-2 Spike), driven by a human CMV promoter (hCMV promoter), and with the bovine growth hormone 3′ end poly-adenylation signal (bGH polyA). The pGX0001 backbone includes the kanamycin resistance gene (KanR) and plasmid origin of replication (pUC ori) for production purpose. Those elements are not functional in eukaryotic cells. The original pVAX1 expression vector was obtained from Thermo Fisher Scientific. The map and description of the modified expression vector pVAX1 (pGX0001) are shown in FIG. 1D. Modifications were introduced into pVAX1 to create pGX0001 and are identified based on the reported sequence of pVAX1 available from Thermo Fisher Scientific. These modifications are listed below and no issues have been detected regarding plasmid amplification and antigen transcription and translation. No further changes in the sequence of pGX0001 have been observed to date in any of the plasmid products in the platform using pGX0001 as the backbone.

C>G 241 in hCMV promoter
C>T 1158 backbone, downstream of the bovine growth hormone polyadenylation signal (bGH polyA)
A>- 2092 backbone, downstream of the Kanamycin resistance gene (KanR)
C>T 2493 in pUC origin of replication (pUC ori)
G>C 2969 in very end of pUC Ori upstream of RNASeH site
Base pairs 2, 3 and 4 are changed from ACT to CTG in backbone, upstream of hCMV promoter.

A schematic diagram of pGX9527 is presented in FIG. 1E. pGX9527 includes the following elements:

| Elements: | Base Pairs: |
|---|---|
| hCMV Promoter: | 137-724 |
| SynCon ® SARS-CoV-2 Spike Coding Sequence: | 742-4578 |
| bGH PolyA: | 4622-4846 |
| Kanamycin Resistance gene (KanR): | 5019-5813 |
| pUC Ori: | 6112-6785 |

As shown in FIG. 1E, pGX9527 was made by cloning of the SynCon® SARS-CoV-2 Spike Coding Sequence into pGX0001 at the BamHI and XhoI sites.

Strain-matched spike sequences (pWT, pB.1.351) were similarly optimized and cloned into identical restriction site locations into the pGX0001 backbone. Pseudovirus plasmids were designed and constructed as previously described [Andrade, V. M., et al., INO-4800 DNA Vaccine Induces Neutralizing Antibodies and T cell Activity Against Global SARS-CoV-2 Variants. bioRxiv, 2021: p. 2021 Apr. 14 439719].

### Cell Lines and In Vitro Plasmid Expression

HEK-293T (ATCC® CRL-3216™) and African Green monkey kidney COS-7 (ATCC® CRL-1651™) cell lines were obtained from ATCC (Old Town Manassas, Va.). All cell lines were maintained in DMEM supplemented with 10% fetal bovine serum (FBS) and penicillin-streptomycin.

For in vitro protein expression by Western blot, human embryonic kidney cells, 5.5×10⁵ 293T were transfected with 2.5 μg pDNA in 6-well plates using Lipofectamine 3000 (Invitrogen #L3000015) transfection reagent following the manufacturer's protocol. Transfections were performed in duplicate wells for each plasmid DNA. Forty-eight hours later cell lysates were harvested using Cell Signaling Cell Lysis Buffer (#9803) and duplicate transfection lysates pooled for expression analysis. Proteins were separated on a 4-12% BIS-TRIS gel (ThermoFisher Scientific), then following transfer, blots were incubated with an anti-SARS-CoV spike protein polyclonal antibodies (S1, Sino Biological #40591-T62; S2, Invitrogen #PA1-41165; RBD, Sino Biological #40592-MP01) then visualized with horseradish peroxidase (HRP)-conjugated anti-mouse or anti-rabbit IgG (Bethyl) (GE Amersham). Beta actin was detected using Santa Cruz #SC-47778.

For in vitro RNA expression by qRT-PCR, transfections, RNA purification, cDNA synthesis, and qPCR assay were performed as previously described [Smith, T. R. F., et al., Immunogenicity of a DNA vaccine candidate for COVID-19. Nat Commun, 2020. 11(1): p. 2601; Andrade, V. M., et al., INO-4800 DNA Vaccine Induces Neutralizing Antibodies and T cell Activity Against Global SARS-CoV-2 Variants. bioRxiv, 2021: p. 2021 Apr. 14 439719]. PCR was performed using a single set of primers and probes recognizing the RNA products of all three plasmids (pS-spike forward ATGATCGCCCAGTACACATC (SEQ ID NO: 8), pS-spike reverse CACGCCGATGCCATTAAATC (SEQ ID NO: 9), pS-spike probe AT CACCAGTGGCTGGACATTTGGA (SEQ ID NO: 10)). In a separate reaction, the same quantity of sample cDNA was subjected to PCR using primers and a probe designed (β-actin Forward—GTGACGTGGACATCCGTA AA (SEQ ID NO: 11); β-actin Reverse—CAGGGCAGTAATCTCCTTCTG (SEQ ID NO: 12); β-actin Probe—TACCCTGGCATTGCTGACAGGATG (SEQ ID NO: 13)) for COS-7 cell line β-actin sequences. The primers and probes were synthesized by Integrated DNA Technologies, Inc. and the probes were labeled with 56-FAM and Black Hole Quencher 1.

### In Vivo Immunogenicity

BALB/c mice (6 weeks old, Jackson Laboratory, Bar Harbor, Me.) and Syrian Golden Hamsters (8 weeks old, Envigo, Indianapolis, Ind.) were housed at Acculab (San Diego, Calif.). Mice (n=8/group) received 30 μL intramuscular (IM) injection of 10 μg pDNA immediately followed by electroporation (EP) in the tibialis anterior (TA) muscle on days 0 and 14 of the experiment. Hamsters (n=4/group) received 60 μL IM injection of 90 μg pDNA immediately followed by EP into the TA on days 0, 14 and 236 of the experiment. The CELLECTRA® EP treatment consists of two sets of pulses with 0.2 Amp constant current. Second pulse set is delayed 4 s. Within each set there are two 52 ms pulses with a 198 ms delay between the pulses. Mice were euthanized on day 21 for terminal blood collection and spleens were harvested for cellular assays. Serum was collected from hamsters on days 236 (pre-boost) and 244 (post-boost) by jugular blood collection for pseudovirus-neutralization assay. All animal treatments and procedures were performed at Acculab, and animal testing and research complied with all relevant ethical regulations and studies received ethical approval by the Acculab Institutional Animal Care and Use Committees (IACUC).

### Heterologous Boost with pGX9527 in Syrian Hamsters against Global SARS-CoV-2 Variants

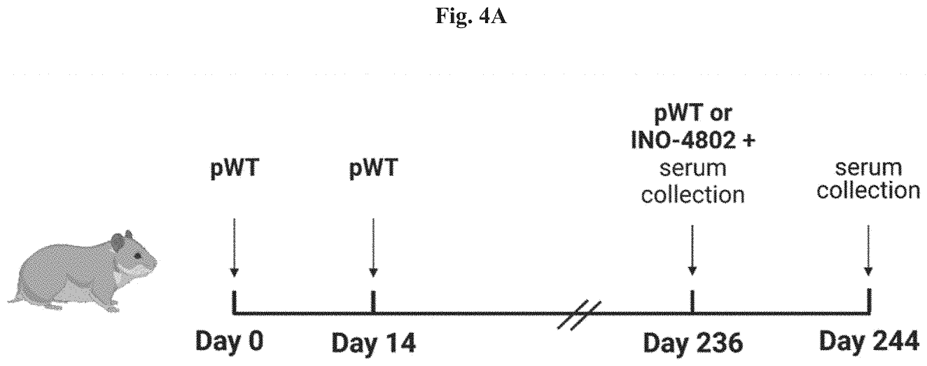

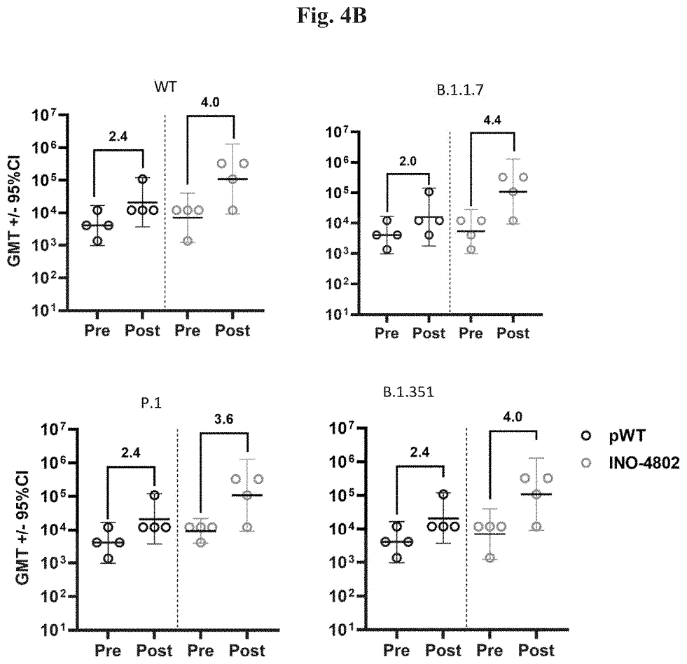

The Syrian Golden hamster is permissible to SARS-CoV-2 infection and is the gold standard small animal model for assessing COVID-19 prophylactics [Baum, A., et al., REGN-COV2 antibodies prevent and treat SARS-CoV-2 infection in rhesus macaques and hamsters. Science, 2020. 370(6520): p. 1110-1115; Chan, J. F., et al., Simulation of the Clinical and Pathological Manifestations of Coronavirus Disease 2019 (COVID-19) in a Golden Syrian Hamster Model: Implications for Disease Pathogenesis and Transmissibility. Clin Infect Dis, 2020. 71(9): p. 2428-2446; Meyer, M., et al., mRNA-1273 efficacy in a severe COVID-19 model: attenuated activation of pulmonary immune cells after challenge. bioRxiv, 2021; Muñoz-Fontela, C., et al., Animal models for COVID-19. Nature, 2020. 586(7830): p. 509-515; Tostanoski, L. H., et al., Ad26 vaccine protects against SARS-CoV-2 severe clinical disease in hamsters. Nat Med, 2020. 26(11): p. 1694-1700.]. This model was employed to test whether pGX9527 could boost the immunity generated by first generation immunogens based on the original wild-type Spike sequence (GenBank RefSeq sequence NC_045512.2 from Wuhan (China)). The immunogenicity of the pWT was tested in hamsters 236 days after receiving 2 doses of the pWT construct (FIG. 4A). Prior to boosting, hamsters were split randomly into two groups of four. The scenario of heterologous (pGX9527) to homologous (pWT construct) boost was compared in terms of magnitude and breadth of humoral responses targeting the VOCs.

### Antigen Binding Assays/ELISAs

Binding ELISAs were performed as described previously (Andrade, V. M., et al., INO-4800 DNA Vaccine Induces Neutralizing Antibodies and T cell Activity Against Global SARS-CoV-2 Variants. bioRxiv, 2021: p. 2021 Apr. 14 439719) except different variants of SARS-CoV-2 S1+S2 spike proteins were used for plate coating. Binding titers were determined after background subtraction of animals vaccinated with mock vector. The S1+S2 wild-type spike protein (Acro Biosystems #SPN-C52H8) contained amino acids 16-1213 of the full spike protein (Accession #QHD43416.1) with R683A and R685A mutations to eliminate the furin cleavage site. The B.1.1.7 and B.1.351, and P.1 S1+S2 variant proteins (Acro Biosystems #SPN-C52H6, #SPN-C52Hc, and #SPN-C52Hg, respectively) additionally contained the following proline substitutions for trimeric protein stabilization: F817P, A892P, A899P, A942P, K986P, and V987P. The B.1.1.7 protein contained the following variant-specific amino acid substitutions: HV69-70del, Y144del, N501Y, A570D, D614G, P681H, T7161, S982A, D1118H; and the B.1.351 protein contained the following substitutions: L18F, D80A, D215G, R2461, K417N, E484K, N501Y, D614G, A701V; and the P.1 protein contained the following:

L18F,T20N,P26S,D138Y,R190S,K417T,E484K,N501Y,D614G,H655Y,T10271,V1176F. Half-area assay plates were coated using 25 μL of 1 μg/mL of protein. Secondary antibodies included IgG (Sigma #A4416), IgG2A (Abcam #ab98698), and IgG1 (Abcam #ab98693) at 1:10,000 dilution.

### Pseudovirus Production

SARS-CoV-2 pseudotyped stocks encoding for the WT, B.1.1.7, P.1, or B.1.351 Spike protein (FIG. 1C) were produced using HEK 293T cells transfected with Lipofectamine 3000 (ThermoFisher) using IgE-SARS-CoV-2 Spike plasmid variants (Genscript) co-transfected with pNL4-3.Luc.R-E-plasmid (NIH AIDS reagent) at a 1:8 ratio. Cell supernatants containing pseudotyped viruses were harvested after 72 h, steri-filtered (Millipore Sigma), and aliquoted for storage at −80° C.

### SARS-CoV-2 Pseudotyped Neutralization

CHO cells stably expressing ACE2 (ACE2-CHOs) to allow permissiveness to SARS-CoV-2 were seeded at 10,000 cells/well. SARS-CoV-2 pseudotyped stocks were titered to yield greater than 30 times the cell only control relative luminescence units (RLU) 72 h post-infection. Sera from vaccinated mice were heat inactivated and serially diluted two-folds starting at 1:16 dilution. Sera were incubated with SARS-CoV-2 pseudotyped virus for 90 min at room temperature. After incubation, sera-pseudovirus mixture was added to ACE2-CHOs and allowed to incubate in a standard incubator (37 degree Celsius, 5% CO₂) for 72 h. After 72 h, cells were lysed using Bright-Glo™ Luciferase Assay (Promega) and RLU was measured using an automated luminometer. Neutralization titers (ID₅₀) were calculated using GraphPad Prism 8 and defined as the reciprocal serum dilution at which RLU were reduced by 50% compared to RLU in virus control wells after subtraction of background RLU in cell control wells.

### COVID-19 Convalescent Serum Samples

Two sets of convalescent donor sera were used in this study, each consisting of 10 donors from the USA. One set included donors that tested positive for SARS-CoV-2 infection in March-April 2020, while the other set consisted donors that tested positive in October 2020. Samples were collected between 14 and 71 days from the onset of symptoms. 15 donors (75%) had disease symptoms classified as mild, 1 donor (5%) was asymptomatic, and 4 donors (20%) had moderate disease symptoms. Seven donors were male, and thirteen were female. Ages ranged from 19 to 60 years. Serum samples were sourced from BioIVT.

### SARS-CoV-2 Spike ELISpot Assay

Mouse Peripheral mononuclear cells (PBMCs) post-vaccination with plasmid were stimulated in vitro with 15-mer peptides (overlapping by 9 amino acids) spanning the full-length Spike protein sequence of the indicated variants. Variant peptide pools (GenScript, custom) included the following changes to match published deletions/mutation in each variant: B.1.1.7 variant (delta69-70, delta144, N501Y, A570D, D614G, P681H, T716I, S982A, D1118H), P.1 variant (L18F, T20N, P26S, D138Y, R190S, K417T, E484K, N501Y, D614G, H655Y, V1176F), and B.1.351 variant (L18F, D80A, D215G, delta242-244, R246I, K417N, E484K, N501Y, D614G, A701V). Cells were incubated overnight in an incubator with peptide pools at a concentration of 1 μg per ml per peptide in a precoated ELISpot plate, (MabTech, Mouse IFNγ ELISpot Plus). Cells were washed off, and the plates were developed via a biotinylated anti-IFN-γ detection antibody followed by a streptavidin-enzyme conjugate resulting in visible spots. Each spot corresponds to an individual cytokine-secreting cell. After plates were developed, spots were scanned and quantified using the CTL S6 Micro Analyzer (CTL) with ImmunoCapture and ImmunoSpot software. Values are shown as the background-subtracted average of measured triplicates. The ELISpot assay qualification determined that 12 spot forming units was the lower limit of detection. Thus, anything above this cutoff is considered to be a signal of an antigen specific cellular response.

### INO-4802 SARS-CoV-2 Spike Flow Cytometry Assays

Mouse splenocytes were also used for intracellular cytokine staining (ICS) analysis and visualized using flow cytometry. One million splenocytes in 200 μL complete RPMI media were stimulated for six hours (37° C., 5% CO₂) with DMSO (negative control), PMA and ionomycin (positive control, 100 ng/mL and 2 μg/mL, respectively), or with the indicated peptide pools (225 ug/mL). Variant peptide pools included the following changes to match published deletions/mutation in each variant: B.1.1.7 variant (delta69-70, delta144, N501Y, A570D, D614G, P681H, T716I, S982A, D1118H), P.1 variant (L18F, T20N, P26S, D138Y, R190S, K417T, E484K, N501Y, D614G, H655Y, V1176F), and B.1.351 variant (L18F, D80A, D215G, K417N, E484K, N501Y, D614G, A701V). After stimulation, cells were washed in PBS for live/dead staining (Life Technologies Live/Dead aqua fixable viability dye), and then stained with extracellular markers. The cells were then fixed and permeabilized (eBioscience™ Intracellular Fixation and Permeabilization Buffer Set) and then stained for the indicated intracellular cytokines using fluorescently conjugated antibodies (Table 1).

TABLE 1

| Tube | Channel | Marker/Cytokine |
|---|---|---|
| 1 | FITC | CD107a |
| 2 | V500 | Live/Dead Fix Aqua |
| 3 | PerCP-Cy5.5 | CD4 |
| 4 | BV786 | CD8 |
| 5 | APC-Cy7 | CD3 |
| 6 | BV605 | IFNγ |
| 7 | AlexaFluor 700 | IL2 |
| 8 | AlexaFluor 647 | IL4 |
| 9 | V450 | TNF |

In a separate flow cytometry assay, circulating T follicular helper (Tfh) cells were assessed in vaccinated mice using a whole blood staining strategy. 100 μL of whole blood was obtained 2 weeks after the second dose of either pGX9527 or pVax. Whole blood was directly stained using the same viability dye and fluorescently conjugated antibodies described above for CD3, CD4, and CD8. The antibody cocktail also included the canonical Tfh markers CXCR5-biotin (BD Biosciences), PD-1 PE-CF594 (BD Biosciences), and ICOS BV650 (BD Biosciences) in the presence of Fc block (BD Biosciences). Following a 45-minute incubation at 4° C., whole blood was lysed using FACS Lysing Solution (BD Biosciences) according to the manufacturer's instructions. Cells were then stained with streptavidin-BV421 (BD Biosciences) for 40 minutes at 4 C, fixed, and acquired on a FACSCelesta flow cytometer (BD Biosciences). Data were analyzed using FlowJo software. Tfh cells were identified as CD4+CXCR5+PD-1+T cells.

### Data Analysis

GraphPad Prism 8.1.2 (GraphPad Software, San Diego, USA) was used for graphical and statistical analysis of data sets. P values of <0.05 were considered statistically significant. A nonparametric Mann-Whitney test was used to assess statistical significance when comparing two groups and a by Kruskal-Wallis test (ANOVA) with Dunn multiple comparisons test when comparing three or more groups.

### Results

### Design Strategy of a pan-SARS-CoV-2 Vaccine

The strategy employed to create a pan-SARS-CoV-2 vaccine candidate is described in a step by step manner (FIG. 1A). SARS-CoV-2 genome sequence entries (derived from GISAID) covering a four-month period (October 2020-January 2021) were collected from multiple geographic regions (Brazil, Canada, India, Italy, Japan, Nigeria, South Africa, United Kingdom, United States) to provide a broadly representative pool of current and emerging variants. Consistent mutations in the SARS-CoV-2 Spike sequences were aggregated for each region. The survey detected large numbers of low frequency mutations which if included wholesale would result in a sequence too divergent from real circulating variants. To prevent this, mutations were manually curated and selected for inclusion. Any low frequency mutation was only considered if it was widespread across multiple geographical locations. The results from each of these regions were then aggregated to determine a common set of overlapping mutations from emerging variations in SARS-CoV-2 Spike protein sequences to generate a single SARS-CoV-2 SynCon® Spike immunogen. Manual sequence inspection and observations derived from spike molecular models informed decisions on number and placement of mutations from the pool of aggregated mutations (FIG. 1J). By design, aggregation of large numbers of mutations that did not naturally co-occur was avoided to reduce the potential of generating novel non-relevant epitopes. For ease of understanding, all mutations and changes are numbered according to the canonical SARS-CoV-2 spike sequence numbering scheme. Additional amino acid changes were added in the receptor binding domain (RBD) of the SynCon® SARS-CoV-2. A tandem proline mutation (K986P/V987P) named “2P” was then added to the SynCon® SARS-CoV-2 Spike to augment protein stability [Pallesen, J., et al., Immunogenicity and structures of a rationally designed prefusion MERS-CoV spike antigen. Proc Natl Acad Sci USA, 2017. 114(35): p. E7348-e7357; Xia, X., Domains and Functions of Spike Protein in Sars-Cov-2 in the Context of Vaccine Design. Viruses, 2021. 13(1).]. The final single construct containing all changes was termed INO-4802. The design strategy results can be visualized using an unrooted phylogenetic tree comparing the spike sequences of INO-4802 to several other constructs used in the studies along with several circulating lineages including multiple VOCs. INO-4802 (pS-Pan) occupies a position in the tree that skews it toward multiple VOCs, but is not identical with any, reflective of its consensus-based derivation (FIG. 1B). Plasmids matched to wild-type (pWT) and B.1.351 (pB.1.351) variants used as controls show identity to the matched Spike glycoprotein sequences as expected. (FIG. 1F).

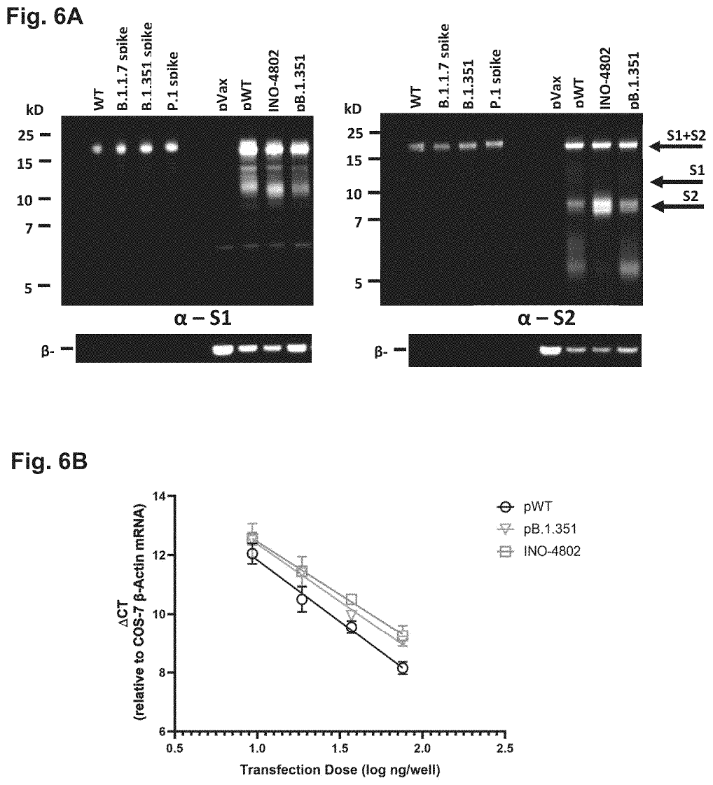

To confirm expression of the immunogens, in vitro Spike protein production in HEK-293T cells after transfection with the corresponding plasmid constructs was measured by Western blot analysis using a cross-reactive antibody against the RBD region of the SARS-CoV-2 Spike (“S”) protein on cell lysates. Western blots of the lysates of HEK-293T cells transfected with pWT (or pS-WT), pB.1.351 (or pS-B.1.351), or pS-Pan (pGX9527, INO-4802) constructs revealed bands approximate to the predicted Spike protein molecular weight, 140-142 kDa, (FIG. 1C). Similar results were observed when using antibodies specific to the S1 or S2 regions of the spike as well (FIG. 6A). Spike transgene RNA expression was also confirmed by RT-PCR analysis of COS-7 cells transfected with pWT, pB.1.351, and INO-4802 plasmids (FIG. 6B). In summary, in vitro studies revealed the expression of the Spike transgene at both the RNA and protein level after transfection of cell lines with all three candidate vaccine constructs.

Vaccination with pGX9527 induces binding and neutralizing antibodies against SARS-CoV-2 variants.

The immunogenicity of each of pWT, pB.1.351, and pGX9527 was evaluated in the BALB/c mouse model. Mice were dosed with 10 μg plasmid DNA (pDNA) on day 0 and 14, and sera samples were collected on day 21 for evaluation.

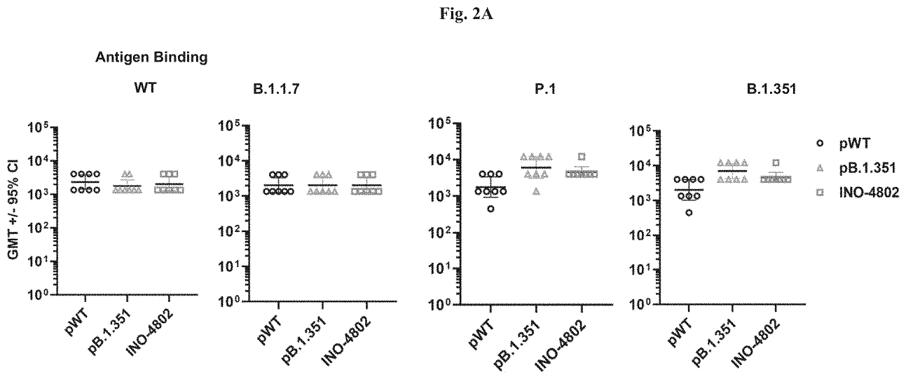

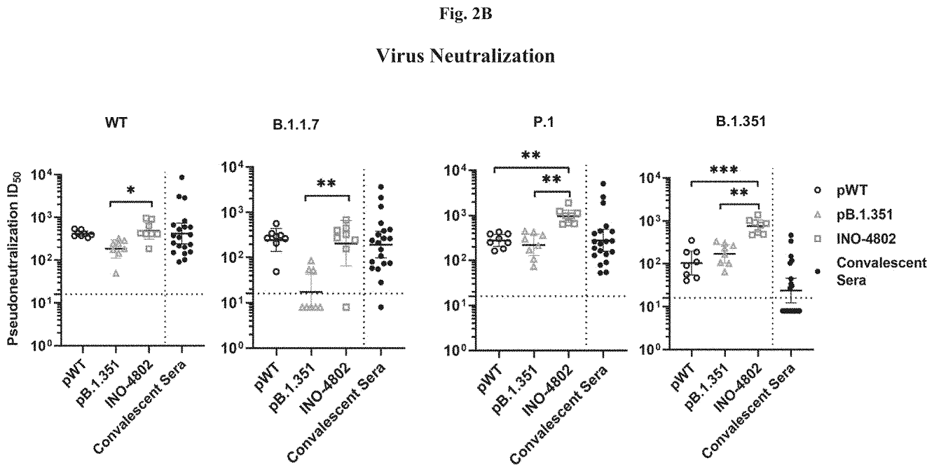

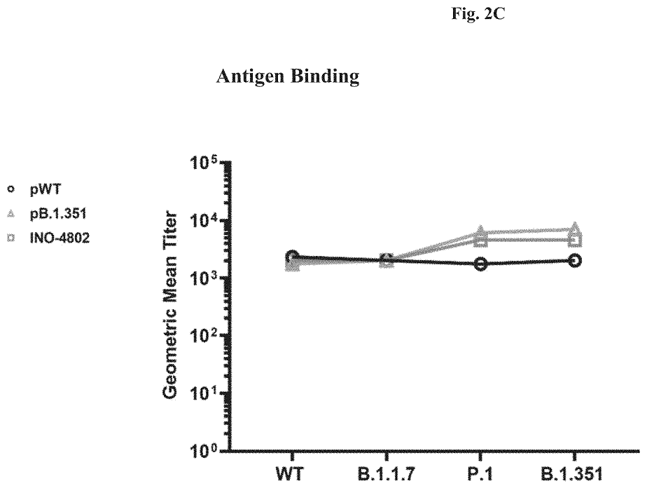

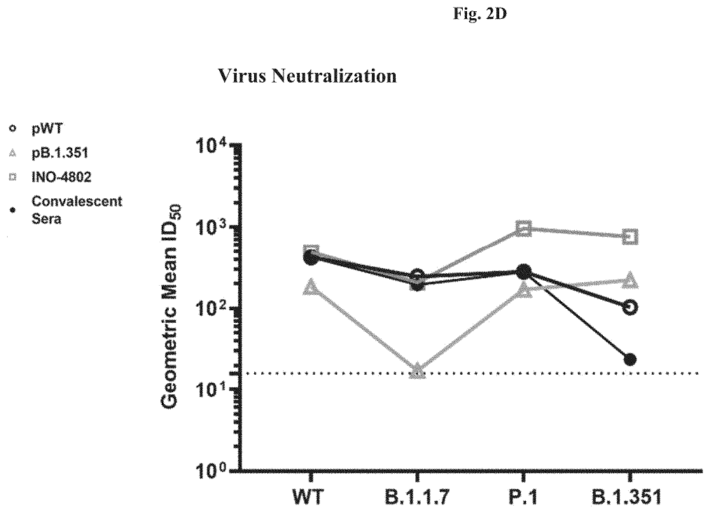

IgG binding titers against the full Spike protein of the WT and variants including B.1.1.7, P.1, and B.1 .351 were evaluated by ELISA. Immunization with pWT, pB.1.351, and pGX9527 induced similar antibody binding titers against the WT and B.1.1.7 variants (FIG. 2A and 2C). Compared to pWT vaccinated animals, there was a small increase in binding titers against P.1 and B.1.351 antigens in mice receiving the pB.1.351 and pGX9527 plasmids.

The functional ability of the antibodies raised in the vaccinated animals immunized with one of the three plasmids to neutralize SARS-CoV-2 was measured. Neutralizing antibody levels against SARS-CoV-2 were measured by a pseudo neutralization assay in the sera of immunized mice (FIGS. 2B and 2D). In the pWT-vaccinated animals, neutralizing activity was similar across WT (425±338-544), B.1.1.7 (288±49-564) and P.1 (295±161-430), but significantly reduced for the B.1.351 (133±40-353) pseudovirus. These results are in line with the other vaccines matched to the WT spike antigen [Garcia-Beltran, W. F., et al., Multiple SARS-CoV-2 variants escape neutralization by vaccine-induced humoral immunity. Cell, 2021; Zhou, D., et al., Evidence of escape of SARS-CoV-2 variant B.1.351 from natural and vaccine-induced sera. Cell, 2021.]. In the animals receiving the matched p.B.1.351 vaccine, neutralizing activity was similar across WT (207.2±49-323), P.1 (261±72.5-445) and B.1.351 (195±64-333), but significantly reduced against B.1.1.7 (29±8-84). In contrast to the matched vaccines, pGX9527-vaccinated mice demonstrated strong neutralizing activity against all variants assessed (548, 317, 816 and 1026, for WT, B.1.1.7, B.1.351 and P.1, respectively). Compared to the matched pWT vaccine, there was a significantly higher neutralizing activity against P.1 and B.1.351 variants in the sera of pGX9527-vaccinated mice. In addition, immunization with pGX9527 showed a significantly higher neutralization titers against all variants, compared to the titers in animals receiving the matched pB.1.351.

Comparison of pGX9527-vaccinated mice sera to human convalescent sera (HCS) collected in 2020 from donors in the United States demonstrates similar neutralizing activity against the WT and B.1.1.7 variants, while neutralizing activity against the P.1 and B.1.351 variants demonstrates enhanced activity in the pGX9527-vaccinated mice sera compared with HCS (P.1 962±703-1317 vs 279±157-495 and B.1.351 765±552 1058 vs 24±12-46 ID50 mean±range for INO-4802 and HCS, respectively).

In summary, pGX9527 demonstrates significantly enhanced neutralizing activity against P.1 and B.1.351 variants while maintaining a strong response against the WT and B.1.1.7 variants, indicating a significant advantage over variant-matched vaccines (FIG. 2D).

### pGX9527 Stimulates T Cell Activity against Global SARS-CoV-2 Variants

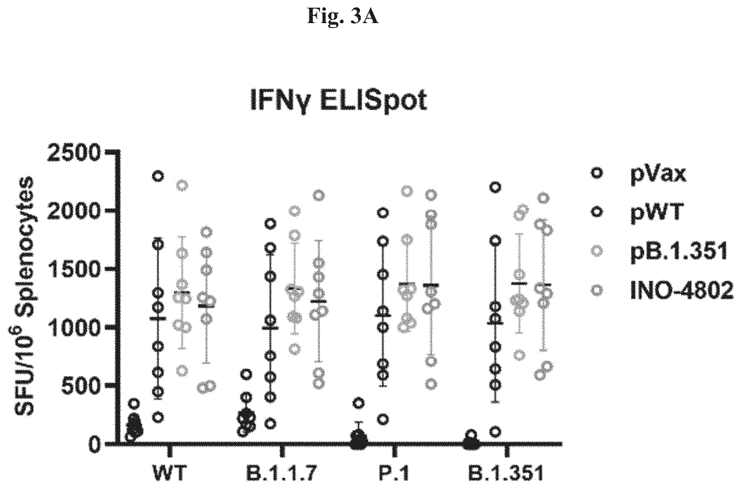

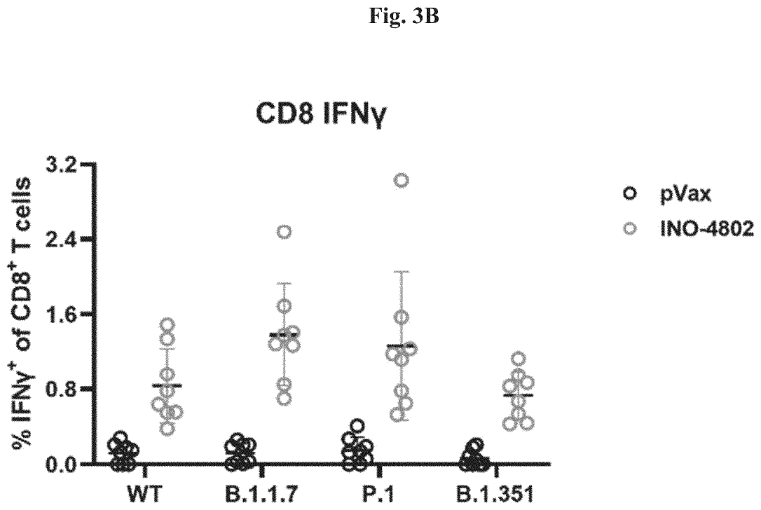

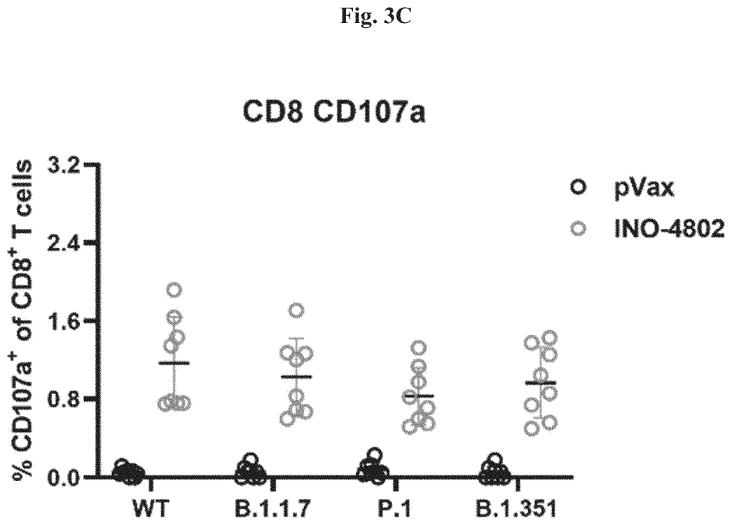

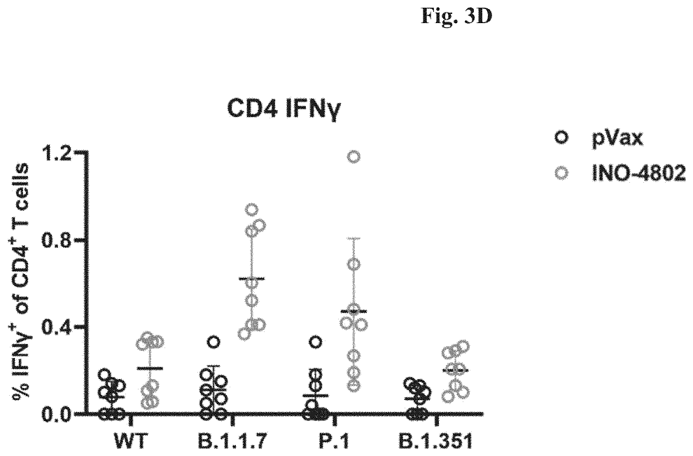

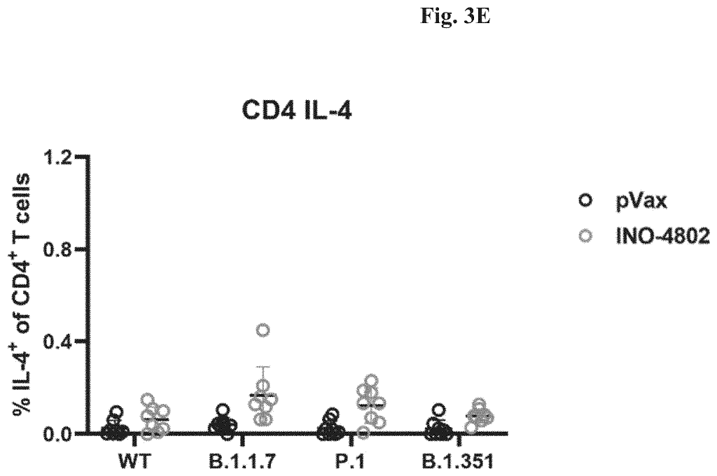

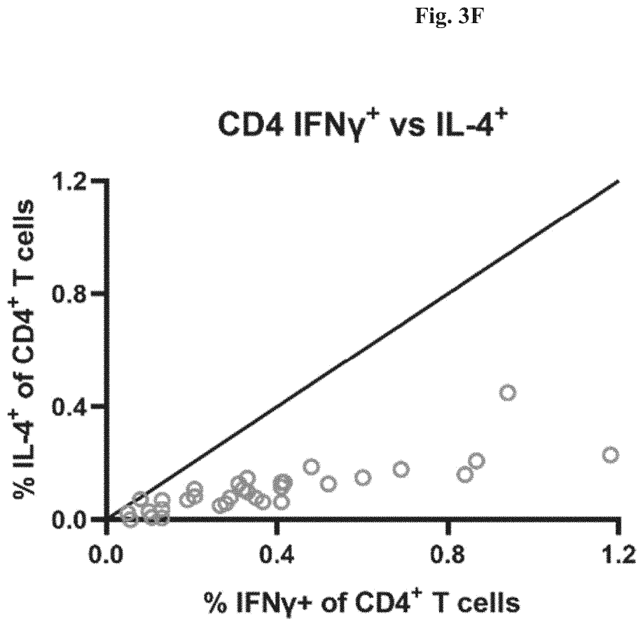

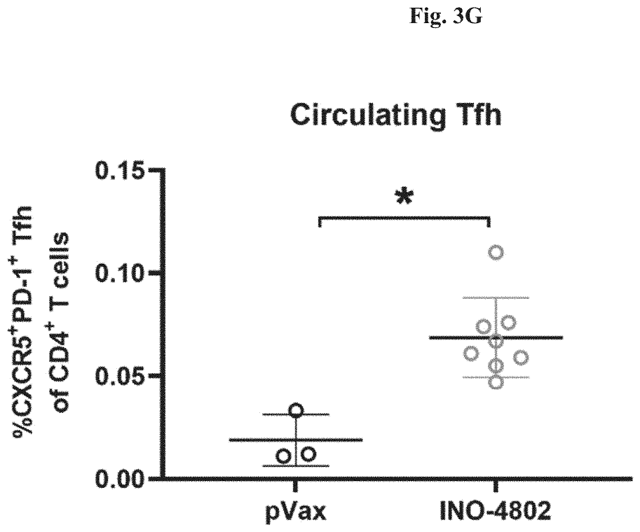

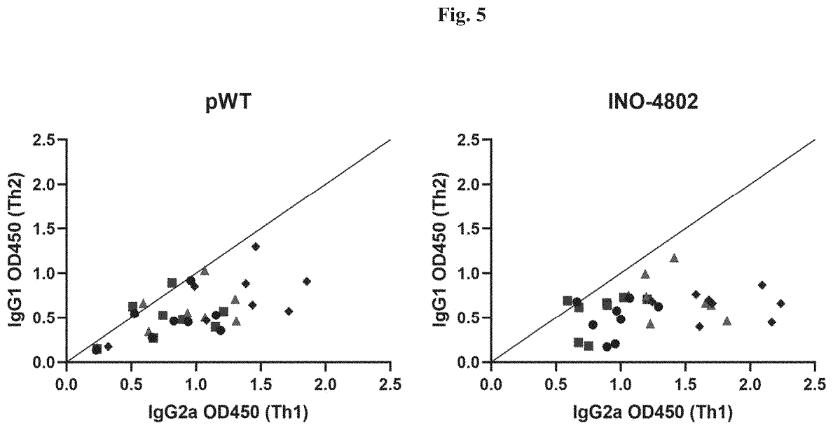

SARS-CoV-2 challenge data in T cell-depleted animals, along with studies indicating reduced disease incidence in individuals harboring pre-existing cross-reactive T cells with SARS-CoV-2 support the importance of COVID-19 vaccines induced cellular immunity [Patel, A., et al., Intradermal delivery of a synthetic DNA vaccine protects macaques from Middle East respiratory syndrome coronavirus. JCI Insight, 2021; Sekine, T., et al., Robust T Cell Immunity in Convalescent Individuals with Asymptomatic or Mild COVID-19. Cell, 2020. 183(1): p. 158-168.e14; Sun, J., et al., Generation of a Broadly Useful Model for COVID-19 Pathogenesis, Vaccination, and Treatment. Cell, 2020. 182(3): p. 734-743.e5.]. T cell responsiveness following vaccination with pGX9527 was thus examined. Splenocytes from mice vaccinated with pWT, pB.1.351, or pGX9527 were stimulated with peptides spanning the WT, B.1.1.7, P.1, and B.1.351 variant Spike proteins. pWT, pB.1.351 and pGX9527 demonstrated induction of T cell responses as measured by IFNγ ELISpot against all variants (FIG. 3A). A similar broad T cell reactivity across SARS-CoV-2 VOC was recently reported in human INO-4800 vaccinees [Andrade, V. M., et al., INO-4800 DNA Vaccine Induces Neutralizing Antibodies and T cell Activity Against Global SARS-CoV-2 Variants. bioRxiv, 2021: p. 2021 Apr. 14 439719.]. The phenotype of CD4 and CD8 T cell responses was characterized by intracellular cytokine staining on splenocytes isolated from pGX9527-vaccinated mice. Antigen-specific T cells producing IFNγ were observed in both CD4 and CD8 compartments (FIGS. 3B and 3D). Additionally, CD8 T cells showed expression of CD107a, a marker of cytolytic potential (FIG. 3C). The balance of T_{H}1 and T_{H}2 expressing cells was evaluated based on cytokine expression profile for T_{H}1 driving IFNγ and T_{H}2 driving IL4 production. CD4 T cells showed greater expression of the canonical T_{H}1 cytokine IFNγ relative to IL-4 (FIGS. 3D-3F), consistent with T_{H}1-skewed T cell responses following pGX9527 vaccination. Further T_{H}1 vs T_{H}2 evaluation was performed by measuring the induction of IgG2A and IgG1 isotype antibodies. ELISA assay results revealed a higher percentage of IgG2A antibodies compared to IgG1 antibodies in animals vaccinated with pWT and pGX9527, indicative of a T_{H}1-biased response (FIG. 5).

Circulating T follicular helper (Tfh) cells are largely representative of a memory CD4 T cell population in the blood that correlates with neutralizing antibody responses, and Tfh cells have been found to be increased in the blood of mice receiving SARS-CoV-2 mRNA vaccines [Crotty, S., T Follicular Helper Cell Biology: A Decade of Discovery and Diseases. Immunity, 2019. 50(5): p. 1132-1148; Locci, M., et al., Human circulating PD-1+CXCR3-CXCR5+ memory Tfh cells are highly functional and correlate with broadly neutralizing HIV antibody responses. Immunity, 2013. 39(4): p. 758-69; Vogel, A. B., et al., BNT162b vaccines protect rhesus macaques from SARS-CoV-2. Nature, 2021. 592(7853): p. 283-289]. Accordingly, it was sought to determine whether Tfh cells were induced following administration of the plasmids. Mice receiving two doses of pGX9527 showed a significantly higher frequency of Tfh cells in circulation relative to mice receiving control pVax (FIG. 3G), suggesting enrichment of circulating Tfh cells at an early timepoint post vaccination.

### Heterologous Boost with pGX9527 in Syrian Hamsters against Global SARS-CoV-2 Variants

The immunogenicity of the pWT was tested in hamsters 236 days after receiving 2 doses of the pWT construct (FIG. 4A). Prior to boosting, hamsters were split randomly into two groups of four. The scenario of heterologous (pGX9527) to homologous (pWT construct) boost was compared in terms of magnitude and breadth of humoral responses targeting the VOCs. VOC antigen binding titers were increased across all variants tested (FIG. 4B). Enhanced levels in the pGX9527 construct group compared to the homologous boost group (pWT GMTs 21044 and pGX9527 109,350) were observed. As the pre-boost levels in the pGX9527 group were slightly higher than the homologous group, the fold changes in binding titers between groups were assessed. A trend towards higher log2-fold increase of endpoint binding titers after boost with pGX9527 (3.6-4.4 log2-fold change) compared to boost with pWT (2.0-2.4 log2 fold change) was observed. These results suggested a broad humoral response of greater magnitude after boosting with the pGX9527 construct compared to homologous construct.

### Discussion

Presented here are the preclinical immunogenicity results for a pan-SARS-CoV-2 DNA vaccine construct (pGX9527) designed with SynCon® technology to provide broad immune response against SARS-CoV-2 Spike antigen on emerging VOCs. In both a primary and heterologous boost vaccine regimen, pGX9527 induced broadly neutralizing antibodies and T cell responses against WT, B.1.1.7, P.1, and B.1.351 SARS-CoV-2 Spike variants in BALB/c mice. In contrast, the cross-neutralizing activity for strain matched vaccines (pWT and pB.1.351) were limited. Importantly, hamsters vaccinated with INO-4802 were completely protected from challenge with B.1.351 (Example 2). Initial data demonstrates the potential for pGX9527 as a pan-SARS-CoV-2 vaccine countermeasure to emerging VOCs.

Current COVID-19 vaccines authorized for emergency use in the US were designed early in the pandemic to match the initial outbreak SARS-CoV-2 strain. The natural mutation rate in RNA viruses inevitably spurred selection for new variants and accumulating evidence points to some critical variants showing enhanced spread, ability to escape known neutralizing antibodies, or leading to higher hospitalization rates [Wibmer, C. K., et al., SARS-CoV-2 501Y.V2 escapes neutralization by South African COVID-19 donor plasma. Nat Med, 2021. 27(4): p. 622-625; Funk, T., et al., Characteristics of SARS-CoV-2 variants of concern B.1.1.7, B.1.351 or P.1: data from seven EU/EEA countries, weeks 38/2020 to 10/2021. Euro Surveill, 2021. 26(16); Volz, E., et al., Assessing transmissibility of SARS-CoV-2 lineage B.1.1.7 in England. Nature, 2021.]. Vaccine trials have already reported reduced efficacy in geographical regions in which the B.1.351 VOC is present [Madhi, S. A., et al., Efficacy of the ChAdOx1 nCoV-19 Covid-19 Vaccine against the B.1.351 Variant. N Engl J Med, 2021; Abdool Karim, S. S. and T. de Oliveira, New SARS-CoV-2 Variants—Clinical, Public Health, and Vaccine Implications. N Engl J Med, 2021; Shinde, V., et al., Preliminary Efficacy of the NVX-CoV2373 Covid-19 Vaccine Against the B.1.351 Variant. medRxiv, 2021: p. 2021 Feb. 25 21252477.]. In response, vaccines matched to B.1.351 have been designed [Wu, K., et al., Variant SARS-CoV-2 mRNA vaccines confer broad neutralization as primary or booster series in mice. bioRxiv, 2021: p. 2021 Apr. 13 439482.]. In addition to the pan-SARS-CoV-2 vaccine construct pGX9527, a DNA vaccine matched to the B.1.351 VOC was tested as a comparator matched VOC strain design. However, while immune responses to the matched antigen were observed, functional antibody responses to other tested variants was reduced (FIG. 2B). These data indicate that focusing on single variant, matched strain approach is to some extent effective but may limit cross-protective potential against diverse novel VOCs, at worst becoming obsolete if facing rapid variant shifts during the development process. This concern is not theoretical. Nearly simultaneous occurrences of B.1.1.7 and B.1.351, followed by P.1 a short time later, show how rapidly the variant landscape can shift. Recent SARS-CoV-2 infection rate surges in India demonstrate the speed at which changes can occur [Mallapaty, S., India's massive COVID surge puzzles scientists. Nature, 2021. 592(7856): p. 667-668.].

In the design process of pGX9527, a broad antigen design strategy using SynCon® technology was utilized as a potential mitigation solution to the limited coverage provided by a matched strain approach. A single construct design consisting of an antigen representative of multiple viral strains while retaining sufficient identity to generate effective and broad immunity is a favorable solution that presents fewer production barriers as defined by cost of goods and formulation complexity. Application of the SynCon® antigen design platform in conjunction with rational design choices allowed generation of a pan-SARS-CoV-2 vaccine design, pGX9527 (FIG. 1A, 1B). This approach was evaluated in terms of the broadness of the immune response induced by pGX9527 against the VOC.

As detailed in this study, humoral immunogenicity results in BALB/c mice demonstrated the consensus-based approach provided broad cross-reactive neutralizing antibody responses against all the VOC tested (FIG. 2B). While the matched pWT construct and pGX9527 performed similarly in neutralization assays against WT and B.1.1.7-matched pseudotyped virus, strain-matched vaccination with pB.1.351 construct showed poor overall performance against B.1.1.7. This may be due to the unique cluster of mutations in the B.1.351 Spike compared to either WT or to pGX9527, making it a less ideal antigen for promoting a B.1.1.7-directed humoral response. B.1.351 has multiple unique changes to the N-terminal domain (NTD) which along with the RBD contains potent neutralization sites [McCallum, M., et al., N-terminal domain antigenic mapping reveals a site of vulnerability for SARS-CoV-2. bioRxiv, 2021]. Generation of neutralizing response to B.1.351 alone may come at the cost of a lack of neutralizing antibody response to more diverse lineages. pGX9527 demonstrated superiority to both pWT and pB.1.351 in inducing functional antibodies against P.1 and B.1.351-matched pseudoviruses (FIG. 2D). The greater magnitude of neutralizing activity induced by pGX9527 compared to pB.1.351 against P.1 VOC may be less surprising than those against B.1.351 VOC, as the pB.1.351 was strain-matched in this case. Added design features in pGX9527 to promote antigen stability, notably the 2P mutation, may be an important structural advantage to account for this observed difference. Comparison of pGX9527-vaccinated mouse sera with COVID-19 HCS demonstrated a stark increase in neutralizing activity against the P.1 and B.1.351 variant highlighting pGX9527's ability to provide broad neutralizing activity against multiple variants.

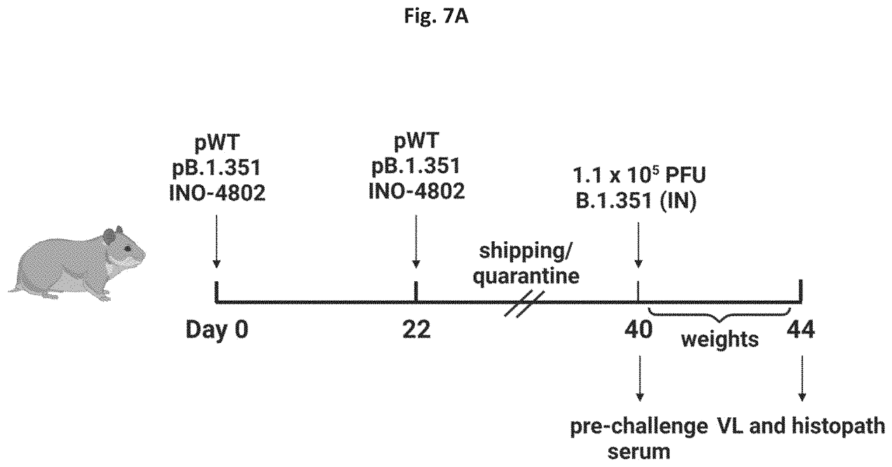

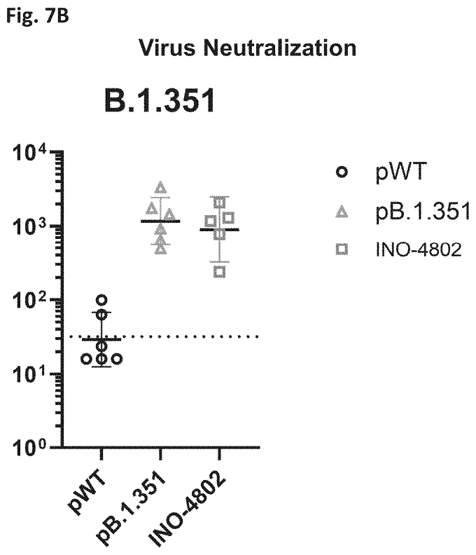

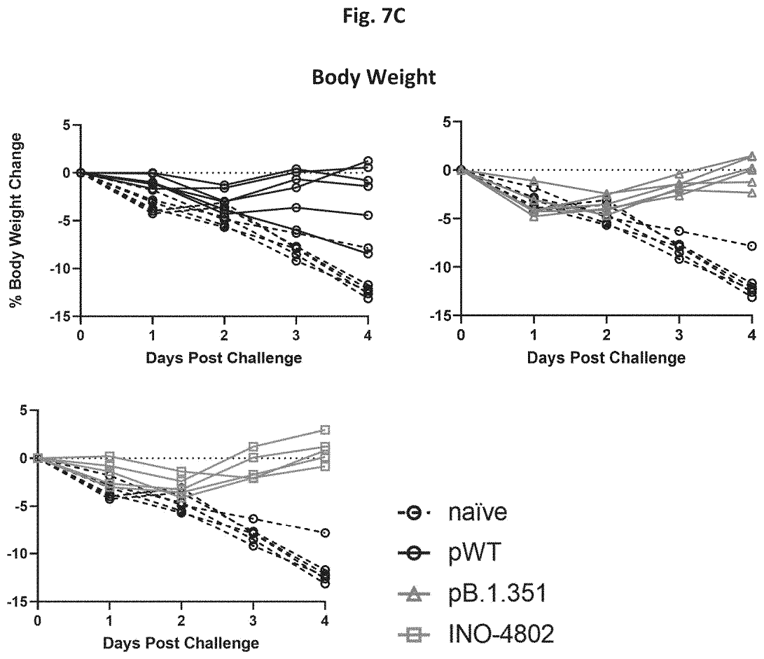

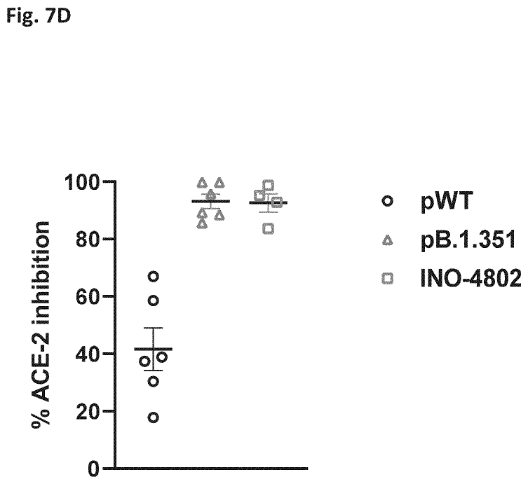

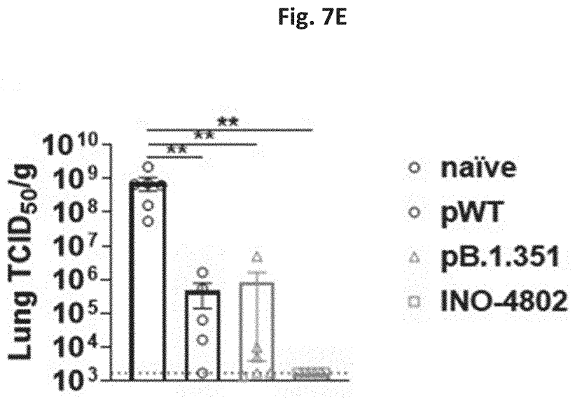

Vaccination with pWT, pB1.351 and pGX9527 resulted in comparably strong IFNγ ELISpot responses against WT and VOC-matched peptide pools in a murine model (FIG. 3A-3G). Lack of differentiation between the vaccine constructs in respect to level of T cell immunity against VOC Spike antigens was expected. The highly diverse and linear epitope dependent T cell compartment is less impacted than the structurally dependent functional antibody response. The results are supported by the maintenance of pWT T cell immunity against the same panel of VOC [Andrade, V. M., et al., INO-4800 DNA Vaccine Induces Neutralizing Antibodies and T cell Activity Against Global SARS-CoV-2 Variants. bioRxiv, 2021: p. 2021 Apr. 14 439719], as well as data in COVID-19 exposed donors and vaccinees showing a negligible impact of SARS-CoV-2 variants on CD4 and CD8 T cell responses [Tarke, A., et al., Negligible impact of SARS-CoV-2 variants on CD4 (+) and CD8 (+) T cell reactivity in COVID-19 exposed donors and vaccinees. bioRxiv, 2021.]. In addition to ELISpot analysis, additional functional T cell assays were performed. pGX9527 vaccination induced CD107a/IFN-γ+ cross-reactive CD8 T cells and Tfh cells, cell populations involved in cytotoxic and augmenting B cell responses, respectively (FIGS. 3B and 3C). Support for an important role of T cells was provided in the SARS-CoV-2 challenge study (FIG. 4). In the B.1.351 SARS-CoV-2 challenge study (Example 2), significant protection against both weight loss and reduction in lung viral loads in animals immunized with the pWT vaccine was observed (FIG. 7A-7E). In contrast to the decline in neutralizing antibodies in pWT immunized animals against B.1.351, the level of T cell activity against the VOCs was fully maintained (FIG. 3A-3G). Such observations support the role of the T cell compartment in providing protection against COVID-19 disease.

In addition to the use of pGX9527 as a prime vaccine, pGX9527 was evaluated in a heterologous boost regimen (FIG. 4A). pGX9527 boosting was assessed approximately 8 months after priming with pWT vaccine in the Syrian Golden hamster model. Delaying the pGX9527 boost by approximately 8 months provided time for the maturation of the immune response from the first round of vaccination and potentially antigenic imprinting to WT spike antigen. However, initial humoral immunogenicity readout suggests strong boost of binding antibody titers across the panel of WT and VOC antigens. The increase in binding titers against all the VOCs tested was greater than same dose boost of pWT.

Pseudotyped virus nAb titers measured here exceed levels reported to correlate with protection in relevant animal models (FIG. 2B & [McMahan, K., et al., Correlates of protection against SARS-CoV-2 in rhesus macaques. Nature, 2021. 590(7847): p. 630-634]).

### Example 2

The Syrian Golden hamster model was employed to assess efficacy of pGX9527 to confer protection against VOC challenge (FIG. 7A).

### Methods

### In Vivo Protection

Syrian Golden Hamsters (8 weeks old, Envigo, Indianapolis, Ind.) (n=6/group) received 100 μL intradermal injection of 95 μg pDNA (pWT, pB.1.351, or pGX9527) immediately followed by EP delivered by CELLECTRA®-3P into the left flank on days 0 and 22 of the experiment. Control animals (n=6) were left untreated. 40 days following the initial pDNA treatment, intranasal inoculation (IN) was performed on Ketamine/Xylazine anesthetized hamsters. The animals were challenged with 5.00×10\hat{ }6 TCID50/10,875,0 PFU (Bioqual SARS-CoV-2 RSA P4 Lot: 020521-105) (B.1.351), with a total volume of 100 μL per animal (50 μL/nostril). Post-challenge, the animals were weighed daily, beginning the day of challenge. Serum was collected from hamsters on day 40 (pre-challenge) and 44 (post-challenge) by jugular blood collection for pseudovirus-neutralization assay. 4 days post challenge animals were euthanized, and lung tissue was collected for measurement of viral loads and histopathological evaluation.

### TCID50 Assay for Determination of Infectious Viral Load

For infectious titer determination from lungs samples, TCID50 assay was performed on the lung tissues harvested from the hamsters. To set up the assay, frozen lung tissue is placed in 15 mL conical tube on wet ice containing 0.5 mL media and homogenized 10-30 secs (Probe, Omni International: 32750H). The tissue homogenate is spun to remove debris at 2000 g ,4° C. for 10 min. The supernatant is passed through a strainer that is placed on original vial, placing vials on wet ice. 20 μL of this supernatant is tested in the assay in quadruplicate in a 96 well plate format.

To perform this assay, Vero TMPRSS2 cells are plated at 25,000 cells per well in DMEM+10% FBS+Gentamicin. The plate is incubated at 37° C., 5.0% CO₂. The cells should be 80-100% confluent the following day. When the 80-100% is confirmed, the media is aspirated out and replaced with 180 μL of DMEM+2% FBS+gentamicin. Then 20 μL of the sample is added to top row in quadruplicate. The top row is mixed 5 times with a pipette and titer down 20 μL, representing 10-fold dilutions. The pipette tips are disposed of between each row and the mixing is repeated until the last row on the plate. The plates for the samples are incubated again at 37° C., 5.0% CO₂ for 4 days. After 4 days, visually inspect for CPE. Non-infected wells will have a clear confluent cell layer. Infected cells will have cell rounding. The presence of CPE is recorded as a plus (+) and absence of CPE as minus (−). The TCID50 is then calculated using the Read-Muench formula.

### Pseudovirus Production

SARS-CoV-2 pseudotyped stocks encoding for the WT, B.1.1.7, P.1, or B.1.351 Spike protein (Table 2) were produced using HEK 293T cells transfected with Lipofectamine 3000 (ThermoFisher) using IgE-SARS-CoV-2 S plasmid variants (Genscript) co-transfected with pNL4-3.Luc.R-E-plasmid (NIH AIDS reagent) at a 1:8 ratio. Cell supernatants containing pseudotyped viruses were harvested after 72 h, steri-filtered (Millipore Sigma), and aliquoted for storage at −80° C.

TABLE 2

| Pseudoviral | |
|---|---|
| construct | Mutations |
| WT | Original Wuhan sequence ref Genbank (NC_045512) |
| B.1.1.7 | Δ69-70/Δ144/N501Y/A570D/D614G/P681H/ |
| | T716I/S982A/D1118H |
| P.1 | L18F/T20N/P26S/D138Y/R190S/K417T/E484K/ |
| | N501Y/D614G/H655Y/T1027I/V1176F |
| B.1.351 | L18F/D80A/D215G/Δ242-244/R246I/K417N/ |
| | E484K/N501Y/D614G/A701V |

### SARS-CoV-2 Pseudotyped Neutralization

CHO cells stably expressing ACE2 (ACE2-CHOs) to allow permissiveness to SARS-CoV-2, were seeded at 10,000 cells/well. SARS-CoV-2 pseudotyped stocks were titered to yield greater than 30 times the cell only control relative luminescence units (RLU) 72 h post-infection. Sera from vaccinated mice were heat inactivated and serially diluted two-folds starting at 1:16 dilution. Sera were incubated with SARS-CoV-2 pseudotyped virus for 90 min at room temperature. After incubation, sera-pseudovirus mixture was added to ACE2-CHOs and allowed to incubate in a standard incubator (37 degree Celsius, 5% CO₂) for 72 h. After 72 h, cells were lysed using Bright-Glo™ Luciferase Assay (Promega) and RLU was measured using an automated luminometer. Neutralization titers (ID50) were calculated using GraphPad Prism 8 and defined as the reciprocal serum dilution at which RLU were reduced by 50% compared to RLU in virus control wells after subtraction of background RLU in cell control wells.

### Ace2 Neutralization Assay

For this assay, V-Plex COVID-19 Ace2 Neutralization Panel V Kit from MSD was used. The manufacturer's procedure was followed when performing the assay. Briefly, 150 ul/well of Blocker A solution was added to the assay plate. Plate was sealed and incubated for one hour at room temperature with shaking (700 rpm). Hamster sera were diluted 1:30 dilution (2 ul of sera into 58 ul of Diluent 100) on the storage plates. Calibrator Reagent used for obtaining a standard curve was also prepared on the storage plate, by diluting it 1:10 in the Diluent 100 for the highest concentration, and then 1:4 in six subsequent steps in Diluent 100 buffer, and keeping the 8th well for diluent only, which would be used as a blank in the calculations. Assay plate was then washed 3 times with 200 ul/well of 1× MSD wash buffer. 25 ul/well of diluted samples and calibrator were added to the assay plate in duplicate. The assay plate was sealed and incubated for one hour at room temperature with shaking (700 rpm). Sulfo-Tag ACE2 Protein reagent was diluted 1:200 and 25 ul/well was added to each well of the plate. The assay plate was sealed and incubated for one hour at room temperature with shaking (700 rpm) and then washed 3 times with 200 ul/well of 1× MSD wash buffer. 150 ul/well of MSD Gold Read Buffer was added to each well, and plate was immediately read on Meso Sector 600 machine. Data were analyzed using MSD Discovery Workbench Software and % inhibition was calculated as % inhibition=(1−(average sample ECL signal/average ECL signal of diluent only))×100.

### Results

All hamsters receiving two doses of pGX9527 (“INO-4802”) were protected from weight loss after B.1.351 live virus challenge (FIG. 7C). Following initial transient decline in body weight animals began to recover from weight loss beyond day 2 post-challenge. Interestingly, 4 of 5 pGX9527-vaccinated animals exceeded their pre-challenge weights by the end of the 4-day challenge study. In contrast, naïve animals continued to decline in body weight until necropsy.

Serum from pGX9527 (“INO-4802”)-immunized hamsters taken at the time of live virus challenge neutralized both WT (mean ID50 672.2) and B.1.351 pseudovirus in vitro (mean ID50 1121) (FIG. 7B).

As shown in FIG. 7D, serum of vaccinated hamsters inhibits binding of the human host receptor ACE-2 to B.1.351 SARS-CoV-2 spike protein in vitro. Following two immunizations with WT-matched spike pDNA vaccine (pWT), serum of vaccinated hamsters inhibits binding of the human ACE-2 receptor protein to the B.1.351 SARS-CoV-2 spike protein (mean 41.65% inhibition) (FIG. 7D). Binding inhibition of ACE-2 is significantly more efficacious with serum from INO-4802 immunized hamsters (mean 92.59% inhibition) compared to pWT serum (p=0.000729, parametric t-test). Serum of INO-4802 immunized hamsters is as potent as serum of hamsters immunized with the B.1.351-matched spike vaccine (pB.1.351) to inhibit binding of ACE-2 to B.1.351 spike (mean 93.10% inhibition).

### INO-4802 Confers Protection Against VOCs in Syrian Golden Hamsters

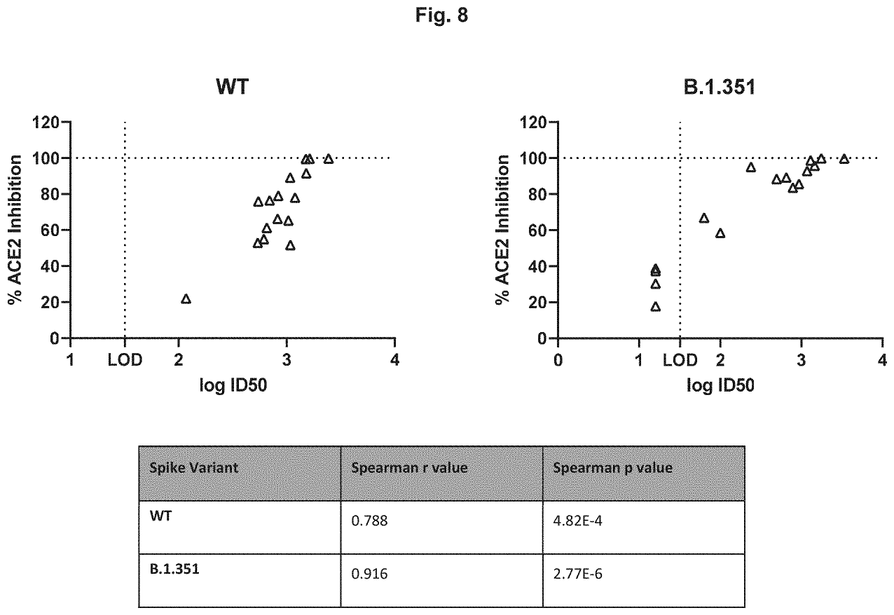

The Syrian Golden hamster model was employed to test whether the INO-4802 vaccine could confer protection against B.1.351 challenge (FIG. 7A). Hamsters receiving two doses of either pB.1.351 or INO-4802 developed high titers of functional antibodies against the B.1.351 variant, as evidenced by strong ACE-2 blocking (FIG. 7D) and pseudovirus neutralizing activity (FIG. 7B). Spearman correlation was performed between the ACE-2 blocking and pseudovirus neutralization assays and showed significant correlation between assays (r=0.916, p=2.77E-06, FIG. 8). Following challenge, vaccinated hamsters showed only a transient decline in body weight and began to recover from weight loss beyond day 2 post-challenge, while naïve animals continued to decline in body weight until necropsy on day 4 (FIG. 7C). Viral titers were undetectable in the lungs of INO-4802-vaccinated hamsters at necropsy (FIG. 7E). Lung viral loads were also significantly reduced in hamsters vaccinated with the pWT and pB.1.351-matched constructs (FIG. 7E). Furthermore, day 44 log TCID50 viral titers in the lung showed significant negative correlation with log ID50 B.1.351 pseudovirus assay results by Spearman correlation (r=−0.543, p=0.027).

Significant protection against both weight loss and reduction in lung viral loads in animals immunized with the pWT vaccine was observed (FIG. 7A-7E).

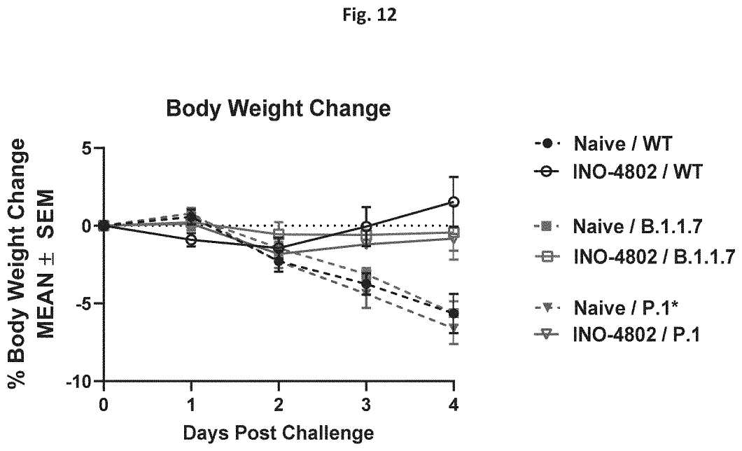

In addition to the B.1.351 variant, the efficacy of INO-4802 protection against WT, B.1.1.7, and P.1 VOCs was tested. As of the time of testing, against all SARS-CoV-2 VOCs tested, maintenance of body weight of INO-4802 vaccinated animals compared to controls was observed (FIG. 12).

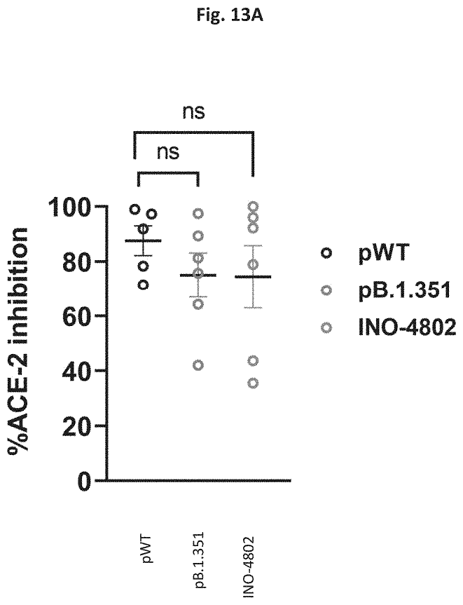

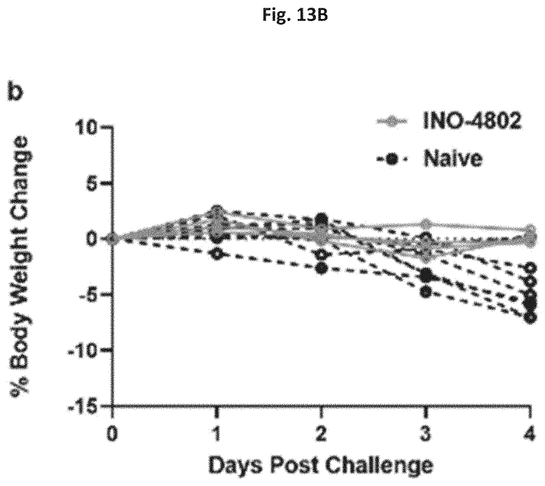

FIGS. 13A and 13B show human ACE2 blocking of B.1.617.2 spike binding by serum from vaccinated hamsters and weight change in hamsters after challenge with B.1.617.2. For FIG. 13A, Syrian Golden Hamsters received IM+EP immunizations with 10 μg pWT, p.B1.351 or INO-4802 on days 0 and 14. Sera collected on day 22 were tested for capacity to block binding of human ACE-2 to B.1.617.2-spike in an electrochemiluminescent-based ELISA assay (mean % inhibition+/−SEM). Not significant (ns) determined by Welch's t test. For FIG. 13B, animals received ID+EP immunizations with 100 μg INO-4802 on days 0 and 21. Hamsters were challenged on day 70 IN with B.1.617.2 and observed for weight loss. Weight change of INO-4802 vaccinated hamsters compared to unvaccinated animals following challenge with B.1.617.2 (Mann-Whitney-test).

### Example 3 Enhanced Immunity to SARS-CoV-2 Variants of Concern Following Prime-Boost Vaccination in Nonhuman Primates

This example evaluates the immunogenicity of a prime-boost regimen in nonhuman primates. Rhesus macaques received primary immunization with INO-4800, a first-generation DNA vaccine matched to SARS-CoV-2 Spike protein of the original strain and currently in clinical development. One year later, the immunized animals were randomized and received either homologous boost with INO-4800 or heterologous boost with INO-4802. Following the boost, all animals showed significantly increased levels of functional antibody responses with neutralizing and ACE2 blocking activity against multiple SARS-CoV-2 VOCs. These data indicate homologous or heterologous prime-boost strategies with the INO-4800 and INO-4802 DNA vaccines enhance broad humoral responses against emerging SARS-CoV-2 variants.

### Materials & Methods

Animals and Immunizations. All rhesus macaque experiments were approved by the Institutional Animal Care and Use Committee at Bioqual (Rockville, Md.), an Association for Assessment and Accreditation of Laboratory Animal Care (AAALAC) International accredited facility. Nine Chinese rhesus macaques (five males, and four females roughly 4 years of age, ranging from 4.48 kg-8.50 kg) were randomized prior to injection and were initially immunized with one or two 1 mg injections of the SARS-CoV-2 DNA vaccine INO-4800 drug product at weeks 0 and 4 by a minimally invasive intradermal electroporation (ID-EP) administration using the CELLECTRA 2000® Adaptive Constant Current Electroporation Device with a 3P array (Inovio Pharmaceuticals). Approximately one year post prime immunization, study animals were randomized and received a boost immunization at 1 mg per dose of either INO-4800 drug product or INO-4802 drug product by ID-EP administration. Sera samples collected at each timepoint were used to evaluate binding titers, pseudovirus neutralization, intracellular cytokine staining (ICS), and ACE2 blocking activity and to isolate peripheral blood mononuclear cells (PBMC) and serum.

### Peripheral Blood Mononuclear Cell Isolation and IFN-γ Enzyme-Linked Immunospot (ELISpot)

Blood was collected from each study animal into sodium citrate cell preparation tubes (CPT, BD Biosciences). The tubes were centrifuged to separate plasma and lymphocytes, according to the manufacturer's protocol. Samples from the prime immunization were transported by same-day shipment on cold-packs from Bioqual to The Wistar Institute, and boost samples were shipped overnight to Inovio Pharmaceuticals for PBMC isolation. PBMCs were washed, and residual red blood cells were removed using ammonium-chloride-potassium (ACK) lysis buffer. Cells were counted using a ViCell counter (Beckman Coulter) and resuspended in RPMI 1640 (Corning), supplemented with 10% fetal bovine serum (Seradigm), and 1% penicillin/streptomycin (Gibco). Fresh cells were then plated for IFNγ ELISpot assay to detect cellular responses.

Monkey IFN-γ ELISpotPro plates (Mabtech, Sweden, Cat #3421M-2APW-10) were prepared according to the manufacturer's protocol. Freshly isolated PBMCs were added to each well at 200,000 cells per well in the presence of either 1) SARS-CoV-2-specific peptide pools, 2) R10 with DMSO (negative control), or 3) anti-CD3 positive control (Mabtech, 1:1000 dilution), in triplicate. Plates were incubated overnight at 37° C., 5% CO₂, then after a minimum incubation of 18 hours, plates were developed according to the manufacturer's protocol. Spots were imaged using a CTL Immunospot plate reader and antigen-specific responses determined by subtracting the R10-DMSO negative control wells from the wells stimulated with peptide pools.

Antigen-binding ELISA. Nunc plates were coated with 1 ug/mL recombinant SARS-CoV-2 S1+S2 spike proteins and binding titers were determined after background subtraction of animals vaccinated with mock vector. For prime immunization samples, ninety-six well immunosorbent plates (NUNC) were coated with 1 μg/mL recombinant SARS-CoV-2 S1+S2 ECD protein (Sino Biological 40589-V08B1), S1 protein (Sino Biological 40591-V08H), S2 protein (Sino Biological 40590-V08B), or receptor-binding domain (RBD) protein (Sino Biological 40595-V05H) in PBS overnight at 4° C. For boost samples, ELISA half-area plates were coated with 1 μg/mL recombinant spike Wild-Type spike protein, Alpha (B.1.1.7), Beta (B.1.351), Gamma (P.1), Delta (B.1.617.2), and Omicron (B.1.1.529) full length spike variant proteins (Acro Biosystems #SPN-C52H8, #SPN-C52Hc, #SPN-C52Hg, #SPN-C52He, and #SPN-C52Hz, respectively). Secondary antibodies included IgG (Bethyl #A140-202P) at 1:50,000, IgG2A (Abcam #ab98698), and IgG1 (Abcam #ab98693) at 1:10,000 dilution. Plates were washed three times with PBS+0.05% Tween20 (PBS-T) and blocked with 3% FBS in PBS-T for 2 hours at room temperature (RT). Sera from vaccinated macaques were serially diluted in PBS-T+1% FBS, added to the washed ELISA plates, and then incubated for 2 hours at RT. Plates were then washed and incubated with an anti-monkey IgG conjugated to horseradish peroxidase (Bethyl A140-202P) 1 hour at RT. Within 30 minutes of development, plates were read at 450 nm using a Biotek Synergy2 plate reader.

### Pseudovirus Neutralization Assay

SARS-CoV-2 pseudovirus stocks encoding for the wild-type (WT), Alpha (B.1.1.7), Beta (P.1), Gamma (B.1.351), Delta (B.1.617.2), or Omicron (B.1.1.529) Spike protein were produced using HEK 293T cells transfected with Lipofectamine 3000 (ThermoFisher) using IgE-SARS-CoV-2 S plasmid variants (Genscript) co-transfected with pNL4-3.Luc.R-E-plasmid (NIH AIDS reagent). To assess neutralizing activity of serum antibodies, CHO cells stably expressing ACE2 (ACE2-CHOs—Creative Biolabs) were used as target cells at 10,000 cells/well. Sera was heat inactivated and serially diluted prior to incubation with the different SARS-CoV-2 variant pseudoviruses. After a 90-minute incubation, sera-pseudovirus mixture was added to ACE2-CHOs, then 72 hours later, cells were lysed using Bright-Glo™ Luciferase Assay (Promega) and RLU was measured using an automated luminometer. Neutralization titers (ID₅₀) were calculated using GraphPad Prism 8 and defined as the reciprocal serum dilution that is reduced by 50% compared to the signal in the infected control wells.

### Flow Cytometry

Thawed, cryopreserved PBMCs were assessed to determine the frequency of circulating T follicular helper (Tfh) using a panel which included the following antibodies: CD3 (BD Biosciences; clone SP34-2), CD4 (BD Biosciences; clone L200), CXCR5 (eBioscience; clone MU5UBEE), and PD-1 (BioLegend; clone EH12.2H7). Tfh cells were identified as CD3+/CD4+/CXCR5+/PD-1+. Samples were acquired on a BD Celesta flow cytometer and analysed using FlowJo software version 10.7 (Treestar Inc.).

### Meso Scale Discovery ACE2 Blocking Assay

Functional antibody responses were also assessed based on inhibition of ACE2 blocking to SARS-CoV-2 Spike protein (and VOC Spike proteins). For these assays, the Meso Scale Discovery (MSD) V-PLEX SARS-CoV-2 ACE2 Neutralization Kit, Panels 5 and 14, were used according to the manufacturer's instructions with the MSD Sector S 600 instrument. Briefly, MSD plates containing SARS-CoV-2 Spike proteins (wildtype, B.1.1.7, B.1.351, P.1, and B.1.617.2) were blocked, washed, and incubated with sera from vaccinated animals at a 1:27 dilution. Plates were then washed and incubated with SULFO-TAG ACE2 and developed according to the manufacturer's protocol. Functional antibody activity was measured as % inhibition of binding of SULFO-TAG ACE2 to Spike protein.

### Peripheral Blood Mononuclear Cell (PBMC) Isolation and Intracellular Cytokine Staining (ICS)

Blood was collected from each study animal into sodium citrate cell preparation tubes (CPT, BD Biosciences). The tubes were centrifuged to separate plasma and lymphocytes, according to the manufacturer's protocol. Samples from the prime immunization were transported by same-day shipment on cold-packs from Bioqual to The Wistar Institute, and boost samples were shipped overnight to Inovio Pharmaceuticals for PBMC isolation. PBMCs were washed, and residual red blood cells were removed using ammonium-chloride-potassium (ACK) lysis buffer. Cells were counted using a ViCell counter (Beckman Coulter) and cryopreserved in 90% fetal bovine serum (FBS)/10% dimethyl sulfoxide (DMSO). For ICS assays, cells were thawed in RPMI 1640 (Corning), supplemented with 10% fetal bovine serum (Seradigm), and 1% penicillin/streptomycin (Gibco).

For ICS, following an overnight rest at 37° C., PBMCs (1×10⁶/sample) were added to each well and stimulated with either 1) SARS-CoV-2-specific peptide pools, 2) R10 with DMSO (negative control), or 3) eBioscience Cell Stimulation Cocktail containing phorbol 12-myristate 13-acetate (PMA) and ionomycin (Invitrogen, 1:1000 dilution) in the presence of GolgiStop™ and GolgiPlug™ (Invitrogen) and anti-CD28/CD49d. Plates were incubated for 6 hours at 37° C., 5% CO₂, washed, and then stained using an antibody cocktail containing anti-CD3 APC-Cy7, anti-CD4 PerCP-Cy5.5, anti-CD8 BV786, and LIVE/DEAD Fixable Aqua Dead Cell Stain (Invitrogen). Cells were then fixed, permeabilized (eBioscience Foxp3/Transcription Factor Fixation/Permeabilization Kit; ThermoFisher), and stained for intracellular cytokines using an antibody cocktail containing anti-IFNγ BV605, anti-IL-2 BV650, and anti-TNFα APC-R700. Cells were then washed, resuspended and acquired on a BD FACS Celesta. Data were analyzed using FlowJo™ v10.7 Software (BD Life Sciences).

### Results

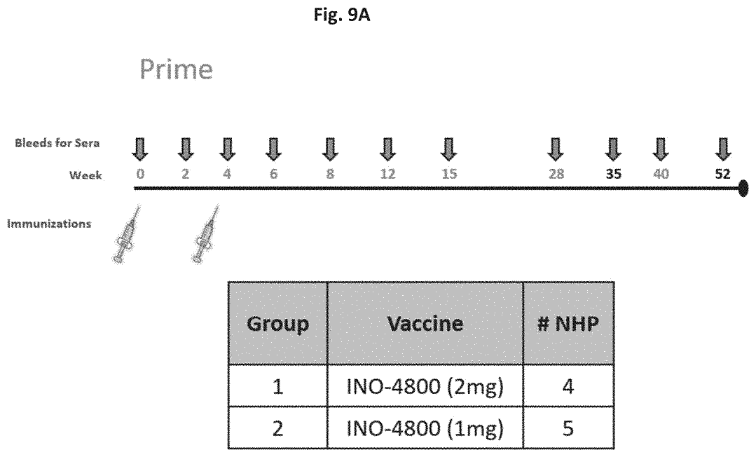

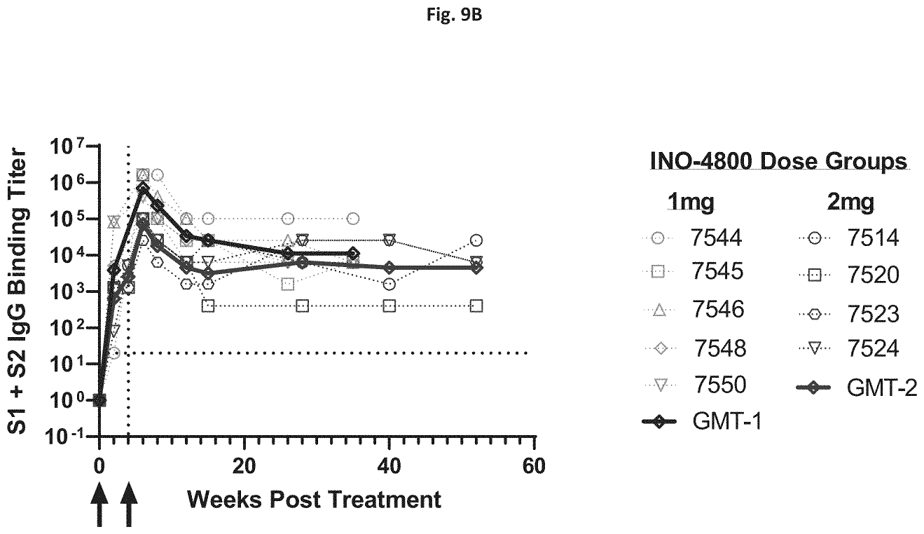

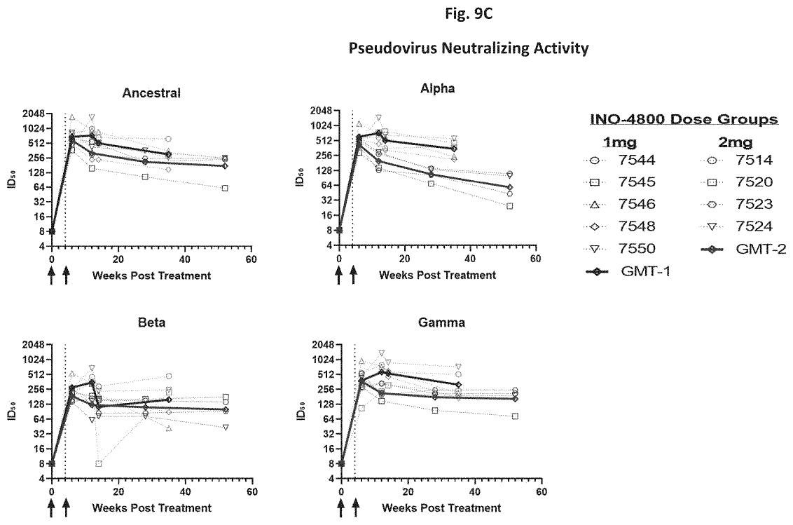

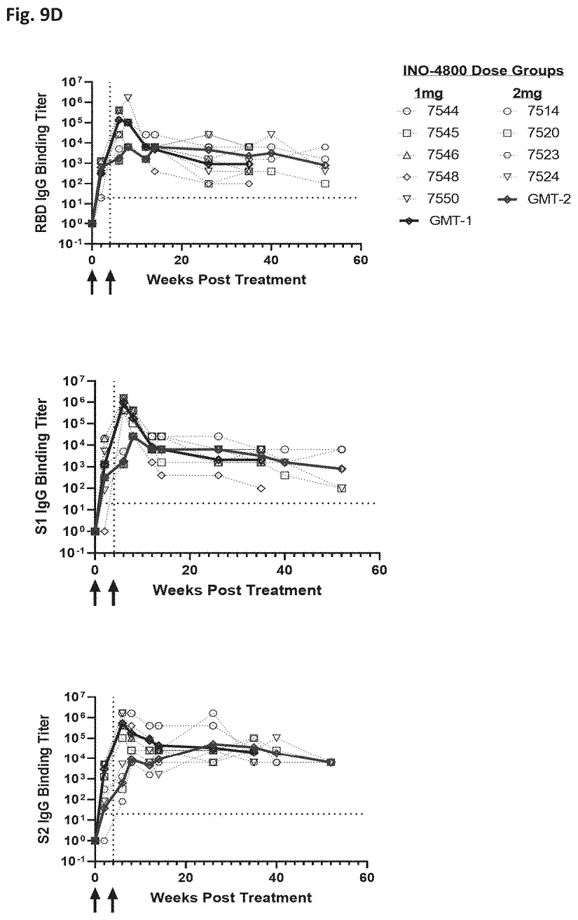

Durability following INO-4800 primary immunization. Initial studies investigated the durability of immune responses in non-human primates (NHPs) primed with INO-4800. NHPs were immunized at week 0 and 4 with either a 1 mg or 2 mg dose of INO-4800, and blood was collected over the course of one year (FIG. 9A). It should be noted that, for FIGS. 9A-9D, the NHPs were initially treated on staggered schedules, and therefore the data from the prime immunization portion of the study show collected data points for NHP IDs #7544, 7545, 7546, 7548, 7550 terminating at Week 35 and for others, IDs #7514, 7520, 7523, 7524, terminating at Week 52. An enzyme-linked immunosorbent assay (ELISA) was used to measure levels of binding antibodies in the serum. Peak antibody titers were observed at week 6 with a geometric mean endpoint titer of 258,032, two weeks following the second immunization (FIG. 9B). Detectable levels of binding antibodies persisted in the serum for the duration of the study, and at the final timepoint prior to boosting, the 1 mg dose group had geometric mean endpoint titers of 11143 for the S1+S2 ECD. The 2 mg dose group had geometric mean endpoint titers of 4525 for the S1+S2 ECD. Similar trends were also observed in the levels of binding antibodies against the SARS-CoV-2 S1, SARS-CoV-2 S2 and RBD proteins (FIG. 9D).

Functional antibody responses were measured in a pseudovirus neutralization assay against the SARS-CoV-2 ancestral, Alpha, Beta and Gamma variants of concern (VOCs) which were in circulation during this time period. Immunization with INO-4800 resulted in the induction of neutralizing antibodies that were increased over baseline for all VOCs (FIG. 9C). SARS-CoV-2 VOC neutralizing antibody responses were durable and remained elevated over baseline at the last collected timepoint, with the 1 mg dose group having a geometric mean titer (GMT) of 301 against ancestral SARS-CoV-2, 349 for Alpha, 158 for Beta, and 317 for Gamma. NHP #7545 showed reduced neutralizing activity at Week 14 for Beta which was attributed to sampling error during plating. The 2 mg dose group had a GMT of 174.6 for the wild-type variant, 58.2 for Alpha, 100.3 for Beta, and 164.2 for Gamma. Together, these data illustrate that the primary INO-4800 vaccination schedule induced SARS-CoV-2 specific antibodies harboring neutralizing activity that were maintained over the period of 35-52 weeks.

### Humoral Responses Following Delivery of Either INO-4800 or INO-4802.

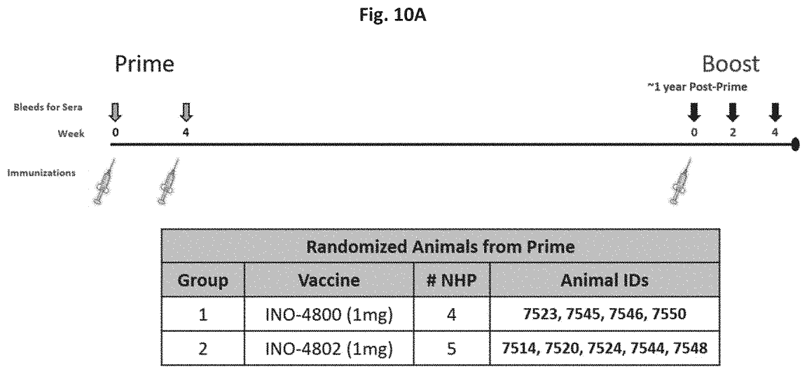

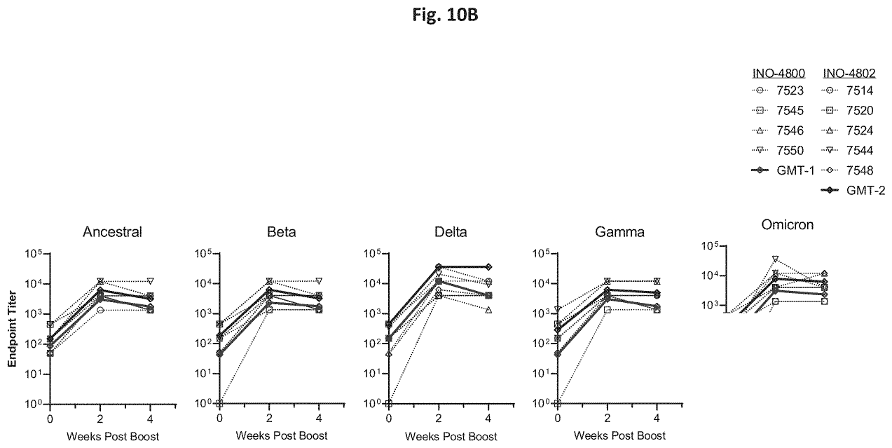

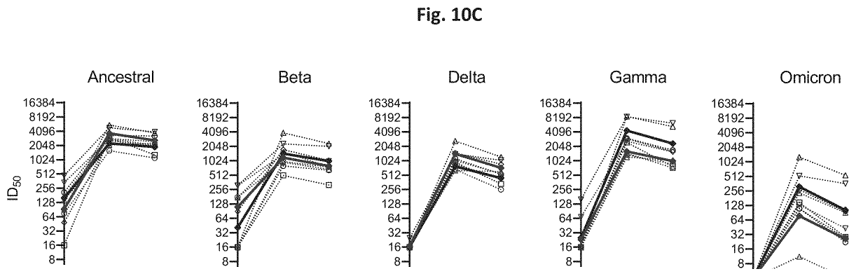

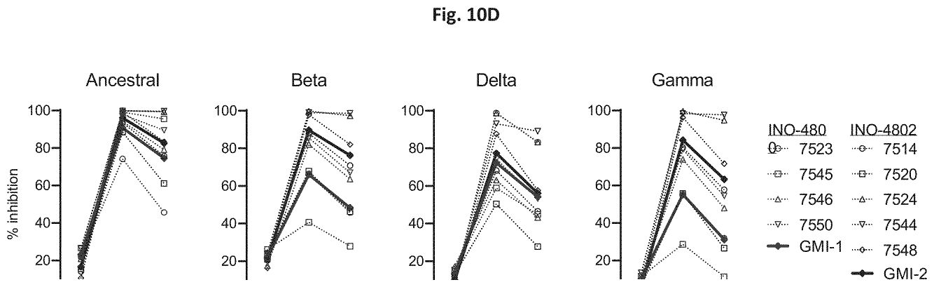

INO-4800 and INO-4802 were evaluated as booster vaccines. The same rhesus macaques that were initially primed with INO-4800 were randomized into two groups and boosted with either INO-4800, homologous to the original vaccine, or INO-4802, an updated pan-SARS-CoV-2 Spike immunogen in a heterologous boost regimen. Rhesus macaques #7544, 7545, 7546, 7548, 7550 were boosted 43 weeks after the initial vaccination while NHPs #7514, 7520, 7523, 7524, were boosted at 64 weeks after the initial vaccination (FIG. 10A).

The homologous boost with INO-4800 resulted in the induction of antibody titers at two weeks post-boost that were increased over pre-boost levels (FIG. 10B). Increases in binding antibody levels showed similar patterns against the ancestral, Beta, Delta, Gamma, and Omicron Spike proteins, with GMTs of 87, 43, 342, 43, and 43, respectively, pre-boost and 3077, 2338, 21044, 3077, and 3077, respectively, post-boost. Likewise, heterologous boost with INO-4802 also led to increased binding antibodies against all variants tested with GMTs of 150, 187, 44, 290, and 187, respectively, pre-boost and 6285, 6285, 6285, 6285, and 7829, respectively, two weeks post-boost for the wild-type, Beta, Delta, Gamma, and Omicron variants (FIG. 10B). Binding titers against any of the variants were not significantly different between INO-4800- and INO-4802-boosted animals at either Week 2 or Week 4.

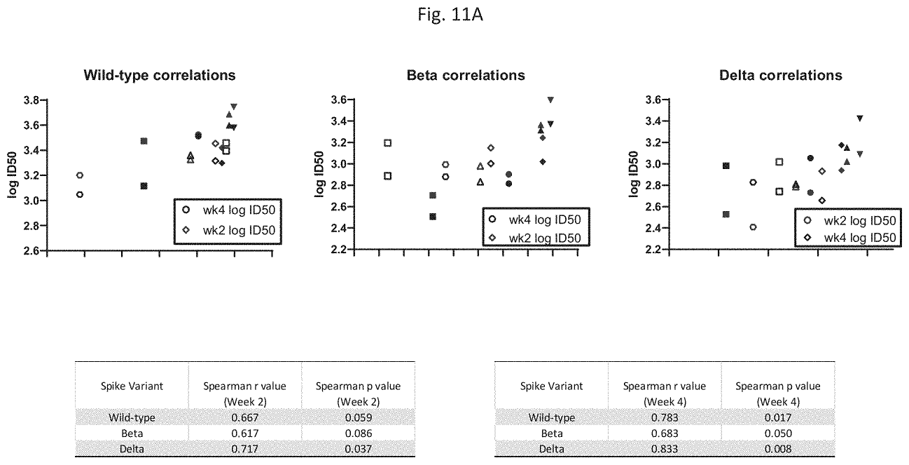

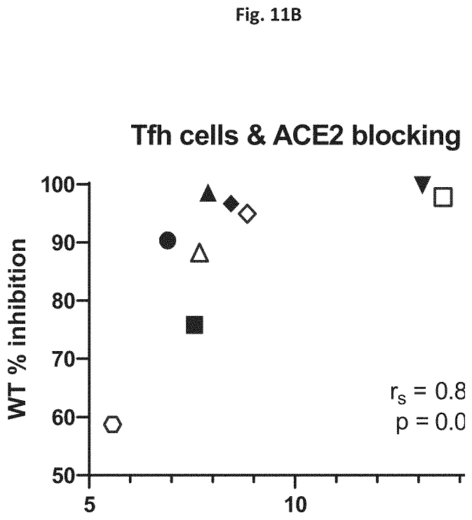

Neutralizing activity against the ancestral, Beta, Delta, Gamma, and Omicron variants was assessed by a pseudovirus neutralization assay, which revealed increased neutralizing antibody responses against all SARS-CoV-2 variants in animals boosted with either INO-4800 or INO-4802 (FIG. 10C). The GMTs at Week 2 for the NHPs after the homologous INO-4800 boost were 2286.2, 1199.3, 785.6, 1596.1, and 78.3 against the ancestral, Beta, Delta, Gamma, and Omicron pseudoviruses, respectively. The GMTs at Week 2 for the NHPs after the heterologous INO-4802 boost were 3712.0, 1452.1, 1434.8, 4389.6, and 312.9 against the ancestral, Beta, Delta, Gamma, and Omicron pseudoviruses, respectively. At Week 2, INO-4802-boosted NHPs showed significantly greater neutralizing activity against the Gamma and Delta pseudoviruses than animals boosted with INO-4800 (P=0.0317 and 0.0317, respectively), although by Week 4, there was not a significant difference between the boost groups. INO-4800- and INO-4802-boosted animals did not show a significant difference in neutralization of the ancestral, Beta, and Omicron pseudoviruses at either timepoint. As an additional readout of functional antibody responses, ACE2/SARS-CoV-2 Spike interaction blocking activity of serum antibodies was measured using a Meso Scale Discovery (MSD) assay, by quantifying the level of inhibition of ACE2 binding to a panel of variant SARS-CoV-2 Spike proteins. In line with the pseudovirus neutralization data, all animals showed an increase in the level of functional anti-SARS-CoV-2 antibodies in their serum following the boost immunization (FIG. 10D). ACE2 blocking activity against any of the variants was not significantly different between INO-4800- and INO-4802-boosted animals at either Week 2 or Week 4 (P=0.4127, 0.0635, 0.7937, and 0.0635 against the ancestral, Beta, Delta, and Gamma VOCs, respectively, at Week 2 and P=0.7302, 0.0635, 0.6032, and 0.1111 against the ancestral, Beta, Delta, and Gamma VOCs, respectively, at Week 4. Positive correlations between pseudovirus neutralization and inhibition of the ACE2/SARS-CoV-2 Spike interaction were observed (FIG. 11A), supporting the overall functional antibody responses observed in animals receiving either booster vaccine.

T follicular helper cells (Tfh) cells were next evaluated. The frequency of circulating Tfh cells positively correlated with ACE2 blocking activity at week 2 in animals boosted with INO-4800 and INO-4802 (FIG. 11B), supporting the generation of functional antibody responses following a boost with SARS-CoV-2 DNA vaccines. Together, these data show an augmentation of humoral responses following a boost with either INO-4800 or INO-4802 in the context of existing SARS-CoV-2 immunity, possibly increasing the breadth of immune response against multiple VOCs.

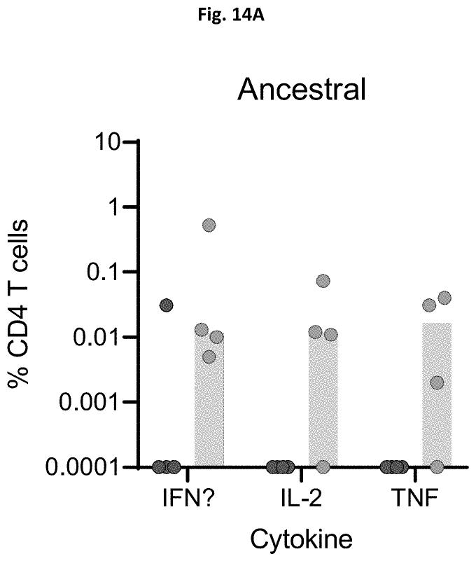

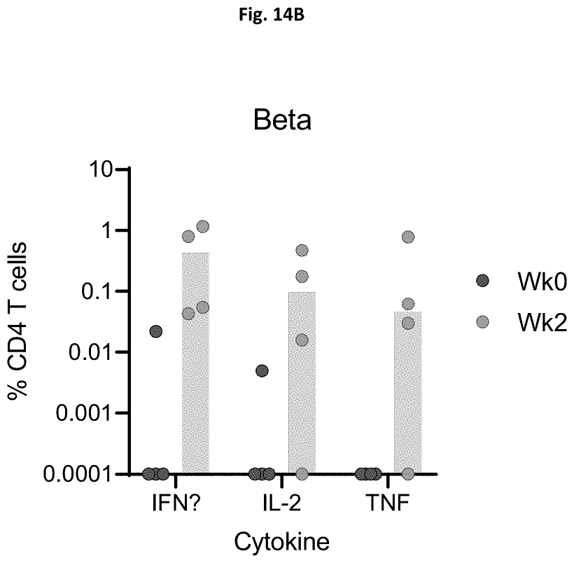

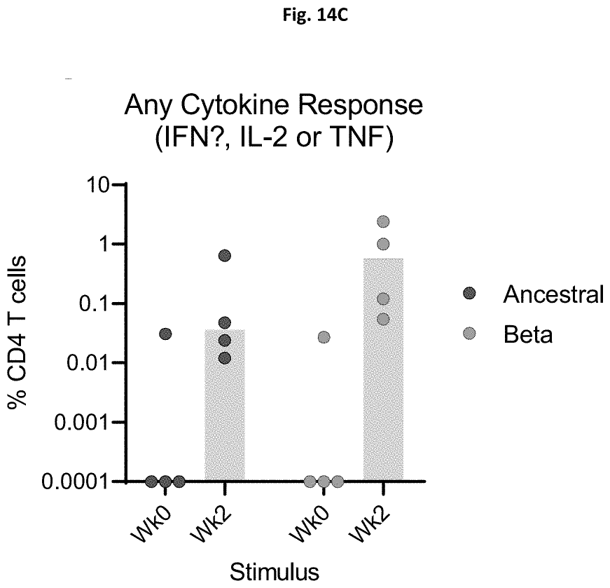

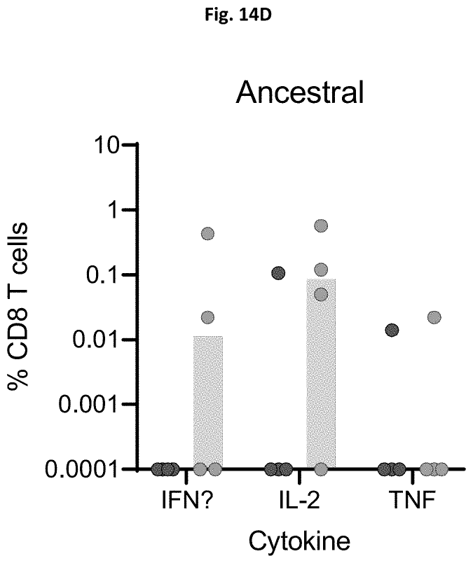

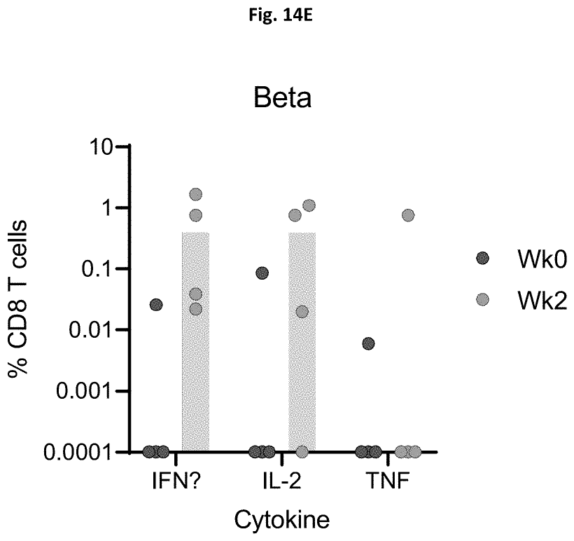

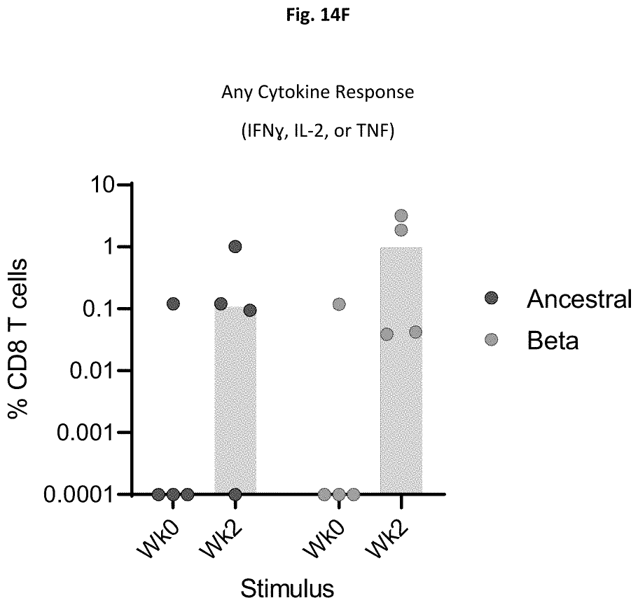

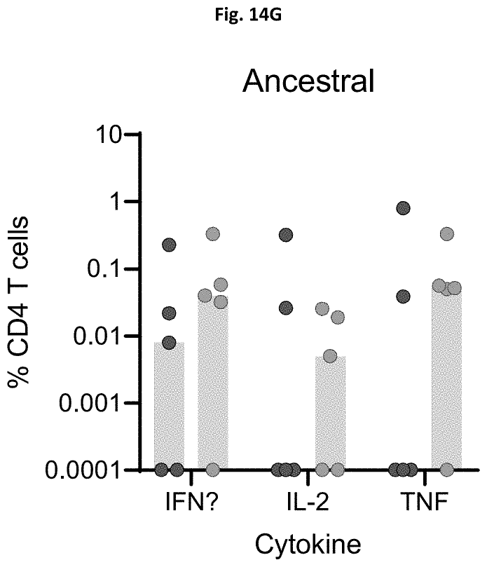

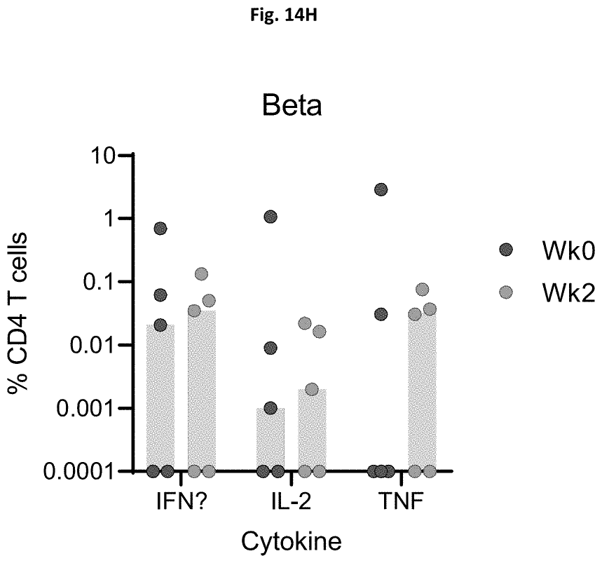

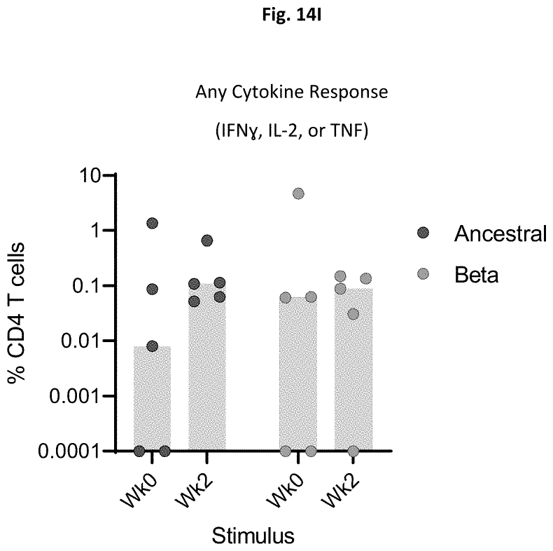

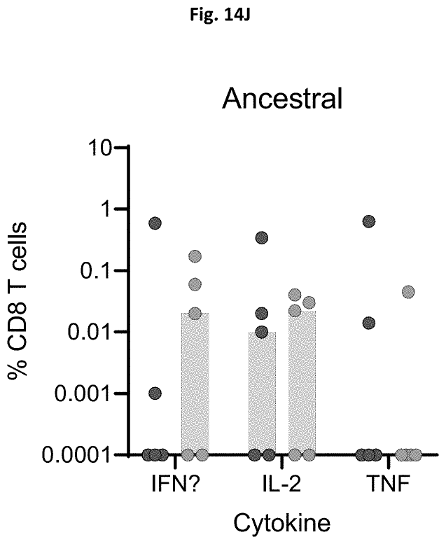

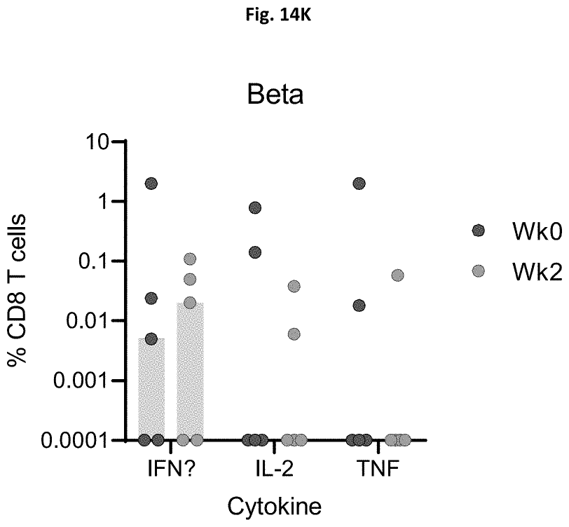

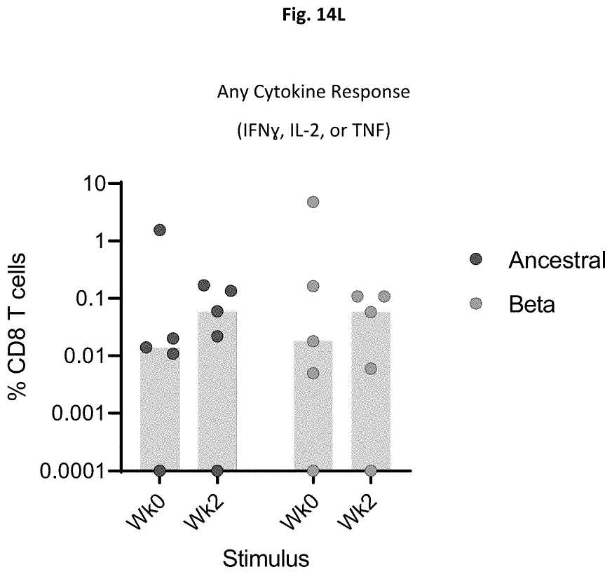

Induction of cellular responses by INO-4800 or INO-4802. Intracellular cytokine staining (ICS) was performed on peripheral blood mononuclear cells (PBMCs) stimulated with peptides matching the ancestral or Beta SARS-CoV-2 Spike proteins to evaluate cellular responses in rhesus macaques boosted with either INO-4800 or INO-4802. Antigen-specific CD4 and CD8 T cell responses were observed in animals boosted with either vaccine (FIGS. 14A-14L). The magnitude of cellular responses was generally greater at 2 weeks post-boost relative to pre-boost levels and showed that boosting with INO-4800 induced CD4 T cell responses that were maintained across the ancestral and Beta variants (FIGS. 14A-14C). Phenotypic analysis of the CD4 T cell responses at Week 2 showed IFNγ secretion in all animals and IL-2 and TNF secretion in 3 of 4 animals (FIGS. 14A, 14B). Similar responses were observed in the CD8 compartment at Week 2, showing secretion of IFNγ (2 of 4 animals for ancestral and 4 of 4 animals for Beta) and IL-2 (3 of 4 animals for each VOC) (FIGS. 14D-14F).

Alternatively, boost with INO-4802 also induced CD4 T cell responses in most animals (FIGS. 14G-14I). Here, CD4 T cell responses against the ancestral and Beta variants at Week 2 were characterized by the secretion of IFNγ (4 of 5 animals and 3 of 5 animals, respectively), IL-2 (3 of 5 animals for both VOCs), and TNF (4 of 5 animals and 3 of 5 animals, respectively) (FIGS. 14G-14H). Most INO-4802-boosted animals also showed responses in the CD8 compartment at Week 2 which were predominantly characterized by the secretion of IFNγ (3 of 5 animals for both VOCs) and IL-2 (3 of 5 animals and 2 of 5 animals, for ancestral and Beta respectively) (FIGS. 14J-14K).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### ILLUSTRATIVE EMBODIMENTS

Embodiment 1. A nucleic acid molecule encoding a Severe Acute Respiratory Syndrome coronavirus 2 (SARS-CoV-2) spike antigen, the nucleic acid molecule comprising:
the nucleic acid sequence of nucleotides 55 to 3831 of SEQ ID NO: 2;
the nucleic acid sequence of SEQ ID NO: 2; or
the nucleic acid sequence of SEQ ID NO: 3.

Embodiment 2. A nucleic acid molecule encoding a SARS-CoV-2 spike antigen, wherein the SARS-CoV-2 spike antigen comprises:
the amino acid sequence set forth in residues 19 to 1277 of SEQ ID NO: 1; or
the amino acid sequence of SEQ ID NO: 1.

Embodiment 3. An expression vector comprising the nucleic acid molecule according to Embodiment 1 or Embodiment 2.

Embodiment 4. The expression vector according to Embodiment 3, wherein the nucleic acid molecule is operably linked to a regulatory element selected from a promoter and a poly-adenylation signal.

Embodiment 5. The expression vector according to Embodiment 3 or Embodiment 4, wherein the vector is a plasmid or viral vector.

Embodiment 6. An immunogenic composition comprising an effective amount of the expression vector according to any one of Embodiments 3-5.

Embodiment 7. The immunogenic composition according to Embodiment 6 further comprising a pharmaceutically acceptable excipient.

Embodiment 8. The immunogenic composition according to Embodiment 7 wherein the pharmaceutically acceptable excipient comprises a buffer, optionally saline-sodium citrate buffer.

Embodiment 9. The immunogenic composition of Embodiment 8, wherein the composition is formulated at a concentration of 10 mg per milliliter of a sodium salt citrate buffer.

Embodiment 10. The immunogenic composition according to any one of Embodiments 6-9, further comprising an adjuvant.

Embodiment 11. A SARS-CoV-2 spike antigen comprising:
the amino acid sequence set forth in residues 19 to 1277 of SEQ ID NO: 1; or
the amino acid sequence of SEQ ID NO: 1.

Embodiment 12. A vaccine for the prevention or treatment of Severe Acute Respiratory Syndrome coronavirus 2 (SARS-CoV-2) infection comprising an effective amount of the nucleic acid molecule of Embodiment 1 or 2, the vector of any one of Embodiments 3-5, or the antigen of Embodiment 11.

Embodiment 13. The vaccine according to Embodiment 12, further comprising a pharmaceutically acceptable excipient.

Embodiment 14. The vaccine according to Embodiment 13, wherein the pharmaceutically acceptable excipient comprises a buffer, optionally sodium salt citrate buffer.

Embodiment 15. The vaccine according to Embodiment 14, formulated at a concentration of 10 mg of nucleic acid per milliliter of a sodium salt citrate buffer.

Embodiment 16. The vaccine according to any one of Embodiments 12 to 15, further comprising an adjuvant.

Embodiment 17. A method of inducing an immune response against Severe Acute Respiratory Syndrome coronavirus 2 (SARS-CoV-2) in a subject in need thereof, the method comprising administering an effective amount of the nucleic acid molecule of Embodiment 1 or 2, the vector of any one of Embodiments 3-5, the immunogenic composition of any one of Embodiments 6-10, the antigen of Embodiment 11, or the vaccine of any one of Embodiments 12-16 to the subject.

Embodiment 18. A method of protecting a subject in need thereof from infection with Severe Acute Respiratory Syndrome coronavirus 2 (SARS-CoV-2), the method comprising administering an effective amount of the nucleic acid molecule of Embodiment 1 or 2, the vector of any one of Embodiments 3-5, the immunogenic composition of any one of Embodiments 6-10, the antigen of Embodiment 11, or the vaccine of any one of Embodiments 12-16 to the subject.

Embodiment 19. A method of protecting a subject in need thereof from a disease or disorder associated with infection with Severe Acute Respiratory Syndrome coronavirus 2 (SARS-CoV-2), the method comprising administering an effective amount of the nucleic acid molecule of Embodiment 1 or 2, the vector of any one of Embodiments 3-5, the immunogenic composition of any one of Embodiments 6-10, the antigen of Embodiment 11, or the vaccine of any one of Embodiments 12-16 to the subject.

Embodiment 20. A method of treating a subject in need thereof against Severe Acute Respiratory Syndrome coronavirus 2 (SARS-CoV-2) infection, the method comprising administering an effective amount of the nucleic acid molecule of Embodiment 1 or 2, the vector of any one of Embodiments 3-5, the immunogenic composition of any one of Embodiments 6-10, the antigen of Embodiment 11, or the vaccine of any one of Embodiments 12-16 to the subject, wherein the subject is thereby resistant to one or more SARS-CoV-2 strains.

Embodiment 21. The method of any one of Embodiments 17 to 20, wherein administering comprises at least one of electroporation and injection.

Embodiment 22. The method of any one of Embodiments 17 to 20, wherein administering comprises parenteral administration followed by electroporation.

Embodiment 23. The method of any one of Embodiments 17 to 22, wherein an initial dose of about 0.5 mg to about 2.0 mg of nucleic acid is administered to the subject, optionally wherein the initial dose is 0.5 mg, 1.0 mg or 2.0 mg of nucleic acid.

Embodiment 24. The method of Embodiment 23, wherein a subsequent dose of about 0.5 mg to about 2.0 mg of nucleic acid is administered to the subject about four weeks after the initial dose, optionally wherein the subsequent dose is 0.5 mg, 1.0 mg or 2.0 mg of nucleic acid.

Embodiment 25. The method of Embodiment 24, wherein one or more further subsequent doses of about 0.5 mg to about 2.0 mg of nucleic acid is administered to the subject at least twelve weeks after the initial dose, optionally wherein the further subsequent dose is 0.5 mg, 1.0 mg, or 2.0 mg of nucleic acid.

Embodiment 26. The method of any one of Embodiments 17 to 25, comprising administering pGX9527, INO-4802 or a biosimilar thereof to the subject.

Embodiment 27. The method of any one of Embodiments 17 to 26, further comprising administering to the subject at least one additional agent for the prevention or treatment of SARS-CoV-2 infection or the treatment or prevention of a disease or disorder associated with SARS-CoV-2 infection, optionally wherein the at least one additional agent comprises a SARS-CoV-2 wild-type-matched vaccine, pGX9501, INO-4800 or a biosimilar thereof.

Embodiment 28. The method of Embodiment 27 wherein the nucleic acid molecule, vector, the immunogenic composition, antigen, or vaccine is administered to the subject before, concurrently with, or after the additional agent.

Embodiment 29. Use of the nucleic acid molecule of Embodiment 1 or 2, the vector of any one of Embodiments 3-5, the immunogenic composition of any one of Embodiments 6-10, the antigen of Embodiment 11, or the vaccine of any one of Embodiments 12-16 in a method of inducing an immune response against Severe Acute Respiratory Syndrome coronavirus 2 (SARS-CoV-2) in a subject in need thereof

Embodiment 30. Use of the nucleic acid molecule of Embodiment 1 or 2, the vector of any one of Embodiments 3-5, the immunogenic composition of any one of Embodiments 6-10, the antigen of Embodiment 11, or the vaccine of any one of Embodiments 12-16 in a method of protecting a subject from infection with Severe Acute Respiratory Syndrome coronavirus 2 (SARS-CoV-2).

Embodiment 31. Use of the nucleic acid molecule of Embodiment 1 or 2, the vector of any one of Embodiments 3-5, the immunogenic composition of any one of Embodiments 6-10, the antigen of Embodiment 11, or the vaccine of any one of Embodiments 12-16 in a method of protecting a subject from a disease or disorder associated with infection with Severe Acute Respiratory Syndrome coronavirus 2 (SARS-CoV-2).

Embodiment 32. Use of the nucleic acid molecule of Embodiment 1 or 2, the vector of any one of Embodiments 3-5, the immunogenic composition of any one of Embodiments 6-10, the antigen of Embodiment 11, or the vaccine of any one of Embodiments 12-16 in a method of treating a subject in need thereof against Severe Acute Respiratory Syndrome coronavirus 2 (SARS-CoV-2) infection.

Embodiment 33. The use of any one of Embodiments 29 to 32 in combination with at least one additional agent for the prevention or treatment of SARS-CoV-2 infection or the treatment or prevention of a disease or disorder associated with SARS-CoV-2 infection, optionally wherein the at least one additional agent comprises a SARS-CoV-2 wild-type matched vaccine, pGX9501, INO-4800 or a biosimilar thereof.

Embodiment 34. The use of any one of Embodiments 29 to 33, wherein the nucleic acid molecule, the vector, the immunogenic composition, the antigen, or the vaccine is administered to the subject by at least one of electroporation and injection.

Embodiment 35. The use of Embodiment 34, wherein the nucleic acid molecule, the vector, the immunogenic composition, the antigen, or the vaccine is parenterally administered to the subject followed by electroporation.

Embodiment 36. The use of any one of Embodiments 29 to 35, wherein an initial dose of about 0.5 mg to about 2.0 mg of nucleic acid is administered to the subject, optionally wherein the initial dose is 0.5 mg, 1.0 mg, or 2.0 mg of nucleic acid.

Embodiment 37. The use of Embodiment 36, wherein a subsequent dose of about 0.5 mg to about 2.0 mg of nucleic acid is administered to the subject about four weeks after the initial dose, optionally wherein the subsequent dose is 0.5 mg, 1.0 mg, or 2.0 mg of nucleic acid.

Embodiment 38. The use of Embodiment 37, wherein a further subsequent dose of about 0.5 mg to about 2.0 mg of nucleic acid is administered to the subject at least twelve weeks after the initial dose, optionally wherein the further subsequent dose is 0.5 mg, 1.0 mg, or 2.0 mg of nucleic acid.

Embodiment 39. The use of any one of Embodiments 29 to 38, wherein the immunogenic composition comprises pGX9527, INO-4802 or a biosimilar thereof.

Embodiment 40. Use of the nucleic acid molecule of Embodiment 1 or 2, the vector of any one of Embodiments 3-5, or the antigen of Embodiment 11 in the preparation of a medicament.

Embodiment 41. Use of the nucleic acid molecule of Embodiment 1 or 2, the vector of any one of Embodiments 3-5, or the antigen of Embodiment 11 in the preparation of a medicament for treating or protecting against infection with Severe Acute Respiratory Syndrome coronavirus 2 (SARS-CoV-2).

Embodiment 42. Use of the nucleic acid molecule of Embodiment 1 or 2, the vector of any one of Embodiments 3-5, or the antigen of Embodiment 11 in the preparation of a medicament for protecting a subject in need thereof from a disease or disorder associated with infection with Severe Acute Respiratory Syndrome coronavirus 2 (SARS-CoV-2).

SEQUENCES

```
SEQ ID NO: 1 SARS-CoV-2 Consensus Spike Antigen amino acid insert sequence of
pGX9527 (IgE leader sequence underlined):
      1 MDWTWILFLV AAATRVHSSQ CVNFTTRTQL PPAYTNSFTR GVYYPDKVFR SSVLHSTQDL
    61 FLPFFSNVTW FHAISGTNGT KRFDNPVLPF NDGVYFASTE KSNIIRGWIF GTTLDSKTQS
  121 LLIVNNATNV VIKVCEFQFC NDPFLGVYYH KNNKSWMESE FRVYSSANNC TFEYVSQPFL
  181 MDLEGKQGNF KNLREFVFKN IDGYFKIYSK HTPINLVRDL PQGFSVLEPL VDLPIGINIT
  241 RFQTLLALHR SYLTPGDSSS GWTAGAAAYY VGYLQPRTFL LKYNENGTIT DAVDCALDPL
  301 SETKCTLKSF TVEKGIYQTS NFRVQPTESI VRFPNITNLC PFGEVFNATR FASVYAWNRK
  361 RISNCVADYS VLYNSASFST FKCYGVSPTK LNDLCFTNVY ADSFVIRGDE VRQIAPGQTG
  421 NIADYNYKLP DDFTGCVIAW NSNNLDSKVG GNYNYLYRLF RKSNLKPFER DISTEIYQAG
  481 STPCNGVKGF NCYFPLQSYG FQPTYGVGYQ PYRVVVLSFE LLHAPATVCG PKKSTNLVKN
  541 KCVNFNFNGL TGTGVLTESN KKFLPFQQFG RDIADTTDAV RDPQTLEILD ITPCSFGGVS
  601 VITPGTNTSN QVAVLYQGVN CTEVPVAIHA DQLTPTWRVY STGSNVFQTR AGCLIGAEHV
  661 NNSYECDIPI GAGICASYQT QTNSHRRARS VASQSIIAYT MSLGAENSVA YSNNSIAIPT
  721 NFTISVTTEI LPVSMTKTSV DCTMYICGDS TECSNLLLQY GSFCTQLNRA LTGIAVEQDK
  781 NTQEVFAQVK QIYKTPPIKD FGGFNFSQIL PDPSKPSKRS FIEDLLFNKV TLADAGFIKQ
  841 YGDCLGDIAA RDLICAQKFN GLTVLPPLLT DEMIAQYTSA LLAGTITSGW TFGAGAALQI
  901 PFAMQMAYRF NGIGVTONVL YENQKLIANQ FNSAIGKIQD SLSSTASALG KLQDVVNQNA
  961 QALNTLVKQL SSNFGAISSV LNDILSRLDP PEAEVQIDRL ITGRLQSLQT YVTQQLIRAA
1021 EIRASANLAA TKMSECVLGQ SKRVDFCGKG YHLMSFPQSA PHGVVFLHVT YVPAQEKNFT
1081 TAPAICHDGK AHFPREGVFV SNGTHWFVTQ RNFYEPQIIT TDNTFVSGNC DWIGIVNNT
1141 VYDPLQPELD SFKEELDKYF KNHTSPDVDL GDISGINASV VNIQKEIDRL NEVAKNLNES
1201 LIDLQELGKY EQYIKWPWYI WLGFIAGLIA IVMVTIMLCC MTSCCSCLKG CCSCGSCCKF
1261 DEDDSEPVLK GVKLHYT
SEQ ID NO: 2 Nucleic acid sequence encoding SARS-CoV-2 Consensus Spike
Antigen amino acid insert sequence of pGX9527 (IgE leader sequence
underlined):
atggattggacttggattctctttctcgttgctgcagccacacgcgttcatagcagcca
gtgtgtgaacttcaccaccagaacacagctgcctcctgcctacaccaacagcttcaccag
aggagtctactacccagacaaagtcttcagaagctctgtgctgcacagcacccaggacct
gttcctgcctttcttcagcaacgtgacctggttccacgccatctctggcaccaacggcac
caagagatttgacaaccctgttcttcctttcaatgatggcgtgtactttgccagcacaga
gaagagcaacatcatccgaggctggatctttggcaccaccctggacagcaaaacccagag
cctgctgatcgtgaacaacgccaccaacgtggtcatcaaggtgtgtgagttccagttctg
caatgaccctttcctgggcgtgtactaccacaagaacaacaagtcctggatggagtctga
gttcagagtctacagctctgccaacaactgcacatttgaatatgtgtcccagcctttcct
gatggacctggagggcaagcagggcaactttaagaacctgagagaatttgtgttcaagaa
catcgatggctacttcaagatctacagcaagcacacacccatcaacctggtgagagacct
gcctcagggcttctctgtgctggagcctctggtggacctgcccatcggcatcaacatcac
cagattccagaccctgctggccctgcacagaagctacctgaccccaggagacagcagcag
cggctggacagctggagctgctgcctactacgtgggctacctgcagcccaggaccttcct
gctgaagtacaacgaaaatggcaccatcacagatgctgttgactgtgccctggaccctct
tagcgagaccaagtgcaccctgaagtccttcacagtggagaaaggcatctaccagaccag
caacttccgagtgcagccaacagagagcatcgtgagatttccaaacatcaccaacctgtg
cccttttggagaagtcttcaatgccaccagatttgcttctgtgtacgcctggaacagaaa
aagaatcagcaactgtgtggctgactactctgtgctgtacaactctgcctccttctccac
cttcaagtgctatggagtctctccaaccaagctgaatgacctgtgcttcaccaacgtgta
tgctgacagctttgtgatcagaggagatgaagtgcggcagattgctcctggccagacagg
caacattgctgactacaactacaagctgcctgatgacttcacaggctgtgtcatcgcctg
gaacagcaacaacctggacagcaaggtgggcggcaactacaactacctgtacagactttt
caggaagagcaacctgaagccttttgaaagagacatctccacagagatctaccaggctgg
cagcacaccctgcaatggtgtgaagggcttcaactgctacttccctctgcagagctacgg
cttccagccaacatatggcgtgggctaccagccttacagagtggtggtgctgtcctttga
gctgctgcacgcccctgccacagtgtgtggccccaagaagagcaccaacctggtgaagaa
caaatgtgtgaacttcaatttcaatggcctgacaggcacaggagtgctgacagagagcaa
caagaagtttcttcctttccagcagtttggaagagacattgctgacaccacagatgctgt
gagagatcctcagaccctggagatcctggatatcacaccctgctcctttggaggagtttc
tgtcatcacacctggcaccaataccagcaaccaagtggctgtgctgtaccaaggagtgaa
ttgcacagaagtgcctgtggccatccacgctgaccagctgacacccacctggagagtgta
cagcacaggcagcaatgttttccagacaagagctggctgcctgattggagcagagcacgt
gaacaacagctatgaatgtgacatccctattggagctggcatctgtgccagctaccagac
ccaaaccaacagccacagaagagccagatctgtggccagccagagcatcatcgcctacac
catgagcctgggagctgagaactctgtggcctacagcaacaacagcatcgccatccccac
caacttcaccatctctgtgaccacagagatcctgcctgtgtccatgaccaagacatctgt
ggactgcaccatgtacatctgtggagacagcacagaatgcagcaacctgctgctgcagta
cggctccttctgcacccagctgaacagagccctgacaggcatcgctgtggagcaggacaa
gaacacacaggaagtgtttgcccaggtgaagcagatctacaaaacaccacccatcaagga
ctttggaggcttcaatttctcccaaatcctgcctgaccccagcaagccttccaagagaag
cttcattgaagacctgctgttcaacaaagtgaccctggctgatgctggcttcatcaagca
gtatggagactgcctgggagacattgctgccagagacctgatctgtgcccagaagtttaa
tggcctgactgtgctgcctcctctgctgacagatgaaatgatcgcccagtacacatctgc
cctgctggctggcaccatcaccagtggctggacatttggagctggagctgccctgcagat
cccttttgccatgcagatggcctacagatttaatggcatcggcgtgacccagaacgtgct
gtacgagaaccagaagctgatcgccaaccagttcaactctgccatcggcaagatccagga
cagcctgagcagcacagcctctgccctgggcaagctgcaggatgtggtgaaccaaaacgc
ccaggccctgaacaccctggtgaagcagctgagcagcaactttggagccatctcctctgt
gctgaatgacatcctgagccggctggaccctccagaagcagaagtgcagatcgacagact
catcacaggccgcctgcagagcctgcagacctacgtgacccagcagctgatcagagctgc
tgagatccgggcctctgccaacctggctgccaccaagatgtcagaatgtgtgctgggcca
gagcaaaagagtggacttctgtggcaaaggctaccacctgatgtccttccctcagtctgc
tcctcacggcgtggtgttcctgcacgtgacctacgtgcctgcccaggagaagaacttcac
cacagctcctgccatctgccacgatggcaaggcccacttcccaagagaaggtgtctttgt
gtccaatggcacccactggttcgtgacccagagaaacttctacgagcctcagatcatcac
cacagacaacacatttgtgtctggcaactgtgatgtggtcatcggcatcgtgaacaacac
agtttatgaccctctgcagcctgagctggacagcttcaaagaagagctggacaagtactt
caagaaccacacatctccagatgtggacctgggagacatctctggcatcaatgcctctgt
ggtgaacatccagaaggaaattgacaggctgaacgaagtggccaagaacctgaacgaaag
cctcatcgacctgcaggagctgggcaagtacgagcagtacatcaagtggccttggtacat
ctggctgggcttcatcgctggcctcatcgccatcgtgatggtgaccatcatgctgtgctg
catgaccagctgctgctcttgcctgaagggctgctgcagctgtggcagctgctgcaagtt
tgatgaagatgactctgagcctgtgctgaagggcgtgaagctgcactacacatgataa
SEQ ID NO: 3 Single strand DNA sequence of pGX9527
      1 gctgcttcgc gatgtacggg ccagatatac gcgttgacat tgattattga ctagttatta
    61 atagtaatca attacggggt cattagttca tagcccatat atggagttcc gcgttacata
  121 acttacggta aatggcccgc ctggctgacc gcccaacgac ccccgcccat tgacgtcaat
  181 aatgacgtat gttcccatag taacgccaat agggactttc cattgacgtc aatgggtgga
  241 gtatttacgg taaactgccc acttggcagt acatcaagtg tatcatatgc caagtacgcc
  301 ccctattgac gtcaatgacg gtaaatggcc cgcctggcat tatgcccagt acatgacctt
  361 atgggacttt cctacttggc agtacatcta cgtattagtc atcgctatta ccatggtgat
  421 gcggttttgg cagtacatca atgggcgtgg atagcggttt gactcacggg gatttccaag
  481 tctccacccc attgacgtca atgggagttt gttttggcac caaaatcaac gggactttcc
  541 aaaatgtcgt aacaactccg ccccattgac gcaaatgggc ggtaggcgtg tacggtggga
  601 ggtctatata agcagagctc tctggctaac tagagaaccc actgcttact ggcttatcga
  661 aattaatacg actcactata gggagaccca agctggctag cgtttaaact taagcttggt
  721 accgagctcg gatccgccac catggattgg acttggattc tctttctcgt tgctgcagcc
  781 acacgcgttc atagcagcca gtgtgtgaac ttcaccacca gaacacagct gcctcctgcc
  841 tacaccaaca gcttcaccag aggagtctac tacccagaca aagtcttcag aagctctgtg
  901 ctgcacagca cccaggacct gttcctgcct ttcttcagca acgtgacctg gttccacgcc
  961 atctctggca ccaacggcac caagagattt gacaaccctg ttcttccttt caatgatggc
1021 gtgtactttg ccagcacaga gaagagcaac atcatccgag gctggatctt tggcaccacc
1081 ctggacagca aaacccagag cctgctgatc gtgaacaacg ccaccaacgt ggtcatcaag
1141 gtgtgtgagt tccagttctg caatgaccct ttcctgggcg tgtactacca caagaacaac
1201 aagtcctgga tggagtctga gttcagagtc tacagctctg ccaacaactg cacatttgaa
1261 tatgtgtccc agcctttcct gatggacctg gagggcaagc agggcaactt taagaacctg
1321 agagaatttg tgttcaagaa catcgatggc tacttcaaga tctacagcaa gcacacaccc
1381 atcaacctgg tgagagacct gcctcagggc ttctctgtgc tggagcctct ggtggacctg
1441 cccatcggca tcaacatcac cagattccag accctgctgg ccctgcacag aagctacctg
1501 accccaggag acagcagcag cggctggaca gctggagctg ctgcctacta cgtgggctac
1561 ctgcagccca ggaccttcct gctgaagtac aacgaaaatg gcaccatcac agatgctgtt
1621 gactgtgccc tggaccctct tagcgagacc aagtgcaccc tgaagtcctt cacagtggag
1681 aaaggcatct accagaccag caacttccga gtgcagccaa cagagagcat cgtgagattt
1741 ccaaacatca ccaacctgtg cccttttgga gaagtcttca atgccaccag atttgcttct
1801 gtgtacgcct ggaacagaaa aagaatcagc aactgtgtgg ctgactactc tgtgctgtac
1861 aactctgcct ccttctccac cttcaagtgc tatggagtct ctccaaccaa gctgaatgac
1921 ctgtgcttca ccaacgtgta tgctgacagc tttgtgatca gaggagatga agtgcggcag
1981 attgctcctg gccagacagg caacattgct gactacaact acaagctgcc tgatgacttc
2041 acaggctgtg tcatcgcctg gaacagcaac aacctggaca gcaaggtggg cggcaactac
2101 aactacctgt acagactttt caggaagagc aacctgaagc cttttgaaag agacatctcc
2161 acagagatct accaggctgg cagcacaccc tgcaatggtg tgaagggctt caactgctac
2221 ttccctctgc agagctacgg cttccagcca acatatggcg tgggctacca gccttacaga
2281 gtggtggtgc tgtcctttga gctgctgcac gcccctgcca cagtgtgtgg ccccaagaag
2341 agcaccaacc tggtgaagaa caaatgtgtg aacttcaatt tcaatggcct gacaggcaca
2401 ggagtgctga cagagagcaa caagaagttt cttcctttcc agcagtttgg aagagacatt
2461 gctgacacca cagatgctgt gagagatcct cagaccctgg agatcctgga tatcacaccc
2521 tgctcctttg gaggagtttc tgtcatcaca cctggcacca ataccagcaa ccaagtggct
2581 gtgctgtacc aaggagtgaa ttgcacagaa gtgcctgtgg ccatccacgc tgaccagctg
2641 acacccacct ggagagtgta cagcacaggc agcaatgttt tccagacaag agctggctgc
2701 ctgattggag cagagcacgt gaacaacagc tatgaatgtg acatccctat tggagctggc
2761 atctgtgcca gctaccagac ccaaaccaac agccacagaa gagccagatc tgtggccagc
2821 cagagcatca tcgcctacac catgagcctg ggagctgaga actctgtggc ctacagcaac
2881 aacagcatcg ccatccccac caacttcacc atctctgtga ccacagagat cctgcctgtg
2941 tccatgacca agacatctgt ggactgcacc atgtacatct gtggagacag cacagaatgc
3001 agcaacctgc tgctgcagta cggctccttc tgcacccagc tgaacagagc cctgacaggc
3061 atcgctgtgg agcaggacaa gaacacacag gaagtgtttg cccaggtgaa gcagatctac
3121 aaaacaccac ccatcaagga ctttggaggc ttcaatttct cccaaatcct gcctgacccc
3181 agcaagcctt ccaagagaag cttcattgaa gacctgctgt tcaacaaagt gaccctggct
3241 gatgctggct tcatcaagca gtatggagac tgcctgggag acattgctgc cagagacctg
3301 atctgtgccc agaagtttaa tggcctgact gtgctgcctc ctctgctgac agatgaaatg
3361 atcgcccagt acacatctgc cctgctggct ggcaccatca ccagtggctg gacatttgga
3421 gctggagctg ccctgcagat cccttttgcc atgcagatgg cctacagatt taatggcatc
3481 ggcgtgaccc agaacgtgct gtacgagaac cagaagctga tcgccaacca gttcaactct
3541 gccatcggca agatccagga cagcctgagc agcacagcct ctgccctggg caagctgcag
3601 gatgtggtga accaaaacgc ccaggccctg aacaccctgg tgaagcagct gagcagcaac
3661 tttggagcca tctcctctgt gctgaatgac atcctgagcc ggctggaccc tccagaagca
3721 gaagtgcaga tcgacagact catcacaggc cgcctgcaga gcctgcagac ctacgtgacc
3781 cagcagctga tcagagctgc tgagatccgg gcctctgcca acctggctgc caccaagatg
3841 tcagaatgtg tgctgggcca gagcaaaaga gtggacttct gtggcaaagg ctaccacctg
3901 atgtccttcc ctcagtctgc tcctcacggc gtggtgttcc tgcacgtgac ctacgtgcct
3961 gcccaggaga agaacttcac cacagctcct gccatctgcc acgatggcaa ggcccacttc
4021 ccaagagaag gtgtctttgt gtccaatggc acccactggt tcgtgaccca gagaaacttc
4081 tacgagcctc agatcatcac cacagacaac acatttgtgt ctggcaactg tgatgtggtc
4141 atcggcatcg tgaacaacac agtttatgac cctctgcagc ctgagctgga cagcttcaaa
4201 gaagagctgg acaagtactt caagaaccac acatctccag atgtggacct gggagacatc
4261 tctggcatca atgcctctgt ggtgaacatc cagaaggaaa ttgacaggct gaacgaagtg
4321 gccaagaacc tgaacgaaag cctcatcgac ctgcaggagc tgggcaagta cgagcagtac
4381 atcaagtggc cttggtacat ctggctgggc ttcatcgctg gcctcatcgc catcgtgatg
4441 gtgaccatca tgctgtgctg catgaccagc tgctgctctt gcctgaaggg ctgctgcagc
4501 tgtggcagct gctgcaagtt tgatgaagat gactctgagc ctgtgctgaa gggcgtgaag
4561 ctgcactaca catgataact cgagtctaga gggcccgttt aaacccgctg atcagcctcg
4621 actgtgcctt ctagttgcca gccatctgtt gtttgcccct cccccgtgcc ttccttgacc
4681 ctggaaggtg ccactcccac tgtcctttcc taataaaatg aggaaattgc atcgcattgt
4741 ctgagtaggt gtcattctat tctggggggt ggggtggggc aggacagcaa gggggaggat
4801 tgggaagaca atagcaggca tgctggggat gcggtgggct ctatggcttc tactgggcgg
4861 ttttatggac agcaagcgaa ccggaattgc cagctggggc gccctctggt aaggttggga
4921 agccctgcaa agtaaactgg atggctttct tgccgccaag gatctgatgg cgcaggggat
4981 caagctctga tcaagagaca ggatgaggat cgtttcgcat gattgaacaa gatggattgc
5041 acgcaggttc tccggccgct tgggtggaga ggctattcgg ctatgactgg gcacaacaga
5101 caatcggctg ctctgatgcc gccgtgttcc ggctgtcagc gcaggggcgc ccggttcttt
5161 ttgtcaagac cgacctgtcc ggtgccctga atgaactgca agacgaggca gcgcggctat
5221 cgtggctggc cacgacgggc gttccttgcg cagctgtgct cgacgttgtc actgaagcgg
5281 gaagggactg gctgctattg ggcgaagtgc cggggcagga tctcctgtca tctcaccttg
5341 ctcctgccga gaaagtatcc atcatggctg atgcaatgcg gcggctgcat acgcttgatc
5401 cggctacctg cccattcgac caccaagcga aacatcgcat cgagcgagca cgtactcgga
5461 tggaagccgg tcttgtcgat caggatgatc tggacgaaga gcatcagggg ctcgcgccag
5521 ccgaactgtt cgccaggctc aaggcgagca tgcccgacgg cgaggatctc gtcgtgaccc
5581 atggcgatgc ctgcttgccg aatatcatgg tggaaaatgg ccgcttttct ggattcatcg
5641 actgtggccg gctgggtgtg gcggaccgct atcaggacat agcgttggct acccgtgata
5701 ttgctgaaga gcttggcggc gaatgggctg accgcttcct cgtgctttac ggtatcgccg
5761 ctcccgattc gcagcgcatc gccttctatc gccttcttga cgagttcttc tgaattatta
5821 acgcttacaa tttcctgatg cggtattttc tccttacgca tctgtgcggt atttcacacc
5881 gcatcaggtg gcacttttcg gggaaatgtg cgcggaaccc ctatttgttt atttttctaa
5941 atacattcaa atatgtatcc gctcatgaga caataaccct gataaatgct tcaataatag
6001 cacgtgctaa aacttcattt ttaatttaaa aggatctagg tgaagatcct ttttgataat
6061 ctcatgacca aaatccctta acgtgagttt tcgttccact gagcgtcaga ccccgtagaa
6121 aagatcaaag gatcttcttg agatcctttt tttctgcgcg taatctgctg cttgcaaaca
6181 aaaaaaccac cgctaccagc ggtggtttgt ttgccggatc aagagctacc aactcttttt
6241 ccgaaggtaa ctggcttcag cagagcgcag ataccaaata ctgttcttct agtgtagccg
6301 tagttaggcc accacttcaa gaactctgta gcaccgccta catacctcgc tctgctaatc
6361 ctgttaccag tggctgctgc cagtggcgat aagtcgtgtc ttaccgggtt ggactcaaga
6421 cgatagttac cggataaggc gcagcggtcg ggctgaacgg ggggttcgtg cacacagccc
6481 agcttggagc gaacgaccta caccgaactg agatacctac agcgtgagct atgagaaagc
6541 gccacgcttc ccgaagggag aaaggcggac aggtatccgg taagcggcag ggtcggaaca
6601 ggagagcgca cgagggagct tccaggggga aacgcctggt atctttatag tcctgtcggg
6661 tttcgccacc tctgacttga gcgtcgattt ttgtgatgct cgtcaggggg gcggagccta
6721 tggaaaaacg ccagcaacgc ggccttttta cggttcctgg ccttttgctg gccttttgct
6781 cacatgttct t
SEQ ID NO: 4 Single strand DNA sequence of pGX9501:
      1 gctgcttcgc gatgtacggg ccagatatac gcgttgacat tgattattga ctagttatta
    61 atagtaatca attacggggt cattagttca tagcccatat atggagttcc gcgttacata
  121 acttacggta aatggcccgc ctggctgacc gcccaacgac ccccgcccat tgacgtcaat
  181 aatgacgtat gttcccatag taacgccaat agggactttc cattgacgtc aatgggtgga
  241 gtatttacgg taaactgccc acttggcagt acatcaagtg tatcatatgc caagtacgcc
  301 ccctattgac gtcaatgacg gtaaatggcc cgcctggcat tatgcccagt acatgacctt
  361 atgggacttt cctacttggc agtacatcta cgtattagtc atcgctatta ccatggtgat
  421 gcggttttgg cagtacatca atgggcgtgg atagcggttt gactcacggg gatttccaag
  481 tctccacccc attgacgtca atgggagttt gttttggcac caaaatcaac gggactttcc
  541 aaaatgtcgt aacaactccg ccccattgac gcaaatgggc ggtaggcgtg tacggtggga
  601 ggtctatata agcagagctc tctggctaac tagagaaccc actgcttact ggcttatcga
  661 aattaatacg actcactata gggagaccca agctggctag cgtttaaact taagcttggt
  721 accgagctcg gatccgccac catggattgg acttggattc tctttctcgt tgctgcagcc
  781 acacgcgttc atagcagcca gtgtgtgaac ctgaccacca gaacacagct gcctcctgcc
  841 tacaccaaca gcttcaccag aggagtctac tacccagaca aagtcttcag aagctctgtg
  901 ctgcacagca cccaggacct gttcctgcct ttcttcagca acgtgacctg gttccacgcc
  961 atccacgtgt ctggcaccaa cggcaccaag agatttgaca accctgttct tcctttcaat
1021 gatggcgtgt actttgccag cacagagaag agcaacatca tccgaggctg gatctttggc
1081 accaccctgg acagcaaaac ccagagcctg ctgatcgtga acaacgccac caacgtggtc
1141 atcaaggtgt gtgagttcca gttctgcaat gaccctttcc tgggcgtgta ctaccacaag
1201 aacaacaagt cctggatgga gtctgagttc agagtctaca gctctgccaa caactgcaca
1261 tttgaatatg tgtcccagcc tttcctgatg gacctggagg gcaagcaggg caactttaag
1321 aacctgagag aatttgtgtt caagaacatc gatggctact tcaagatcta cagcaagcac
1381 acacccatca acctggtgag agacctgcct cagggcttct ctgccctgga gcctctggtg
1441 gacctgccca tcggcatcaa catcaccaga ttccagaccc tgctggccct gcacagaagc
1501 tacctgaccc caggagacag cagcagcggc tggacagctg gagctgctgc ctactacgtg
1561 ggctacctgc agcccaggac cttcctgctg aagtacaacg aaaatggcac catcacagat
1621 gctgttgact gtgccctgga ccctcttagc gagaccaagt gcaccctgaa gtccttcaca
1681 gtggagaaag gcatctacca gaccagcaac ttccgagtgc agccaacaga gagcatcgtg
1741 agatttccaa acatcaccaa cctgtgccct tttggagaag tcttcaatgc caccagattt
1801 gcttctgtgt acgcctggaa cagaaaaaga atcagcaact gtgtggctga ctactctgtg
1861 ctgtacaact ctgcctcctt ctccaccttc aagtgctatg gagtctctcc aaccaagctg
1921 aatgacctgt gcttcaccaa cgtgtatgct gacagctttg tgatcagagg agatgaagtg
1981 cggcagattg ctcctggcca gacaggcaag attgctgact acaactacaa gctgcctgat
2041 gacttcacag gctgtgtcat cgcctggaac agcaacaacc tggacagcaa ggtgggcggc
2101 aactacaact acctgtacag acttttcagg aagagcaacc tgaagccttt tgaaagagac
2161 atctccacag agatctacca ggctggcagc acaccctgca atggtgtgga aggcttcaac
2221 tgctacttcc ctctgcagag ctacggcttc cagccaacaa atggcgtggg ctaccagcct
2281 tacagagtgg tggtgctgtc ctttgagctg ctgcacgccc ctgccacagt gtgtggcccc
2341 aagaagagca ccaacctggt gaagaacaaa tgtgtgaact tcaatttcaa tggcctgaca
2401 ggcacaggag tgctgacaga gagcaacaag aagtttcttc ctttccagca gtttggaaga
2461 gacattgctg acaccacaga tgctgtgaga gatcctcaga ccctggagat cctggatatc
2521 acaccctgct cctttggagg agtttctgtc atcacacctg gcaccaatac cagcaaccaa
2581 gtggctgtgc tgtaccaaga tgtgaattgc acagaagtgc ctgtggccat ccacgctgac
2641 cagctgacac ccacctggag agtgtacagc acaggcagca atgttttcca gacaagagct
2701 ggctgcctga ttggagcaga gcacgtgaac aacagctatg aatgtgacat ccctattgga
2761 gctggcatct gtgccagcta ccagacccaa accaacagcc caagaagagc cagatctgtg
2821 gccagccaga gcatcatcgc ctacaccatg agcctgggag ctgagaactc tgtggcctac
2881 agcaacaaca gcatcgccat ccccaccaac ttcaccatct ctgtgaccac agagatcctg
2941 cctgtgtcca tgaccaagac atctgtggac tgcaccatgt acatctgtgg agacagcaca
3001 gaatgcagca acctgctgct gcagtacggc tccttctgca cccagctgaa cagagccctg
3061 acaggcatcg ctgtggagca ggacaagaac acacaggaag tgtttgccca ggtgaagcag
3121 atctacaaaa caccacccat caaggacttt ggaggcttca atttctccca aatcctgcct
3181 gaccccagca agccttccaa gagaagcttc attgaagacc tgctgttcaa caaagtgacc
3241 ctggctgatg ctggcttcat caagcagtat ggagactgcc tgggagacat tgctgccaga
3301 gacctgatct gtgcccagaa gtttaatggc ctgactgtgc tgcctcctct gctgacagat
3361 gaaatgatcg cccagtacac atctgccctg ctggctggca ccatcaccag tggctggaca
3421 tttggagctg gagctgccct gcagatccct tttgccatgc agatggccta cagatttaat
3481 ggcatcggcg tgacccagaa cgtgctgtac gagaaccaga agctgatcgc caaccagttc
3541 aactctgcca tcggcaagat ccaggacagc ctgagcagca cagcctctgc cctgggcaag
3601 ctgcaggatg tggtgaacca aaacgcccag gccctgaaca ccctggtgaa gcagctgagc
3661 agcaactttg gagccatctc ctctgtgctg aatgacatcc tgagccggct ggacaaggtg
3721 gaagcagaag tgcagatcga cagactcatc acaggccgcc tgcagagcct gcagacctac
3781 gtgacccagc agctgatcag agctgctgag atccgggcct ctgccaacct ggctgccacc
3841 aagatgtcag aatgtgtgct gggccagagc aaaagagtgg acttctgtgg caaaggctac
3901 cacctgatgt ccttccctca gtctgctcct cacggcgtgg tgttcctgca cgtgacctac
3961 gtgcctgccc aggagaagaa cttcaccaca gctcctgcca tctgccacga tggcaaggcc
4021 cacttcccaa gagaaggtgt ctttgtgtcc aatggcaccc actggttcgt gacccagaga
4081 aacttctacg agcctcagat catcaccaca gacaacacat ttgtgtctgg caactgtgat
4141 gtggtcatcg gcatcgtgaa caacacagtt tatgaccctc tgcagcctga gctggacagc
4201 ttcaaagaag agctggacaa gtacttcaag aaccacacat ctccagatgt ggacctggga
4261 gacatctctg gcatcaatgc ctctgtggtg aacatccaga aggaaattga caggctgaac
4321 gaagtggcca agaacctgaa cgaaagcctc atcgacctgc aggagctggg caagtacgag
4381 cagtacatca agtggccttg gtacatctgg ctgggcttca tcgctggcct catcgccatc
4441 gtgatggtga ccatcatgct gtgctgcatg accagctgct gctcttgcct gaagggctgc
4501 tgcagctgtg gcagctgctg caagtttgat gaagatgact ctgagcctgt gctgaagggc
4561 gtgaagctgc actacacatg ataactcgag tctagagggc ccgtttaaac ccgctgatca
4621 gcctcgactg tgccttctag ttgccagcca tctgttgttt gcccctcccc cgtgccttcc
4681 ttgaccctgg aaggtgccac tcccactgtc ctttcctaat aaaatgagga aattgcatcg
4741 cattgtctga gtaggtgtca ttctattctg gggggtgggg tggggcagga cagcaagggg
4801 gaggattggg aagacaatag caggcatgct ggggatgcgg tgggctctat ggcttctact
4861 gggcggtttt atggacagca agcgaaccgg aattgccagc tggggcgccc tctggtaagg
4921 ttgggaagcc ctgcaaagta aactggatgg ctttcttgcc gccaaggatc tgatggcgca
4981 ggggatcaag ctctgatcaa gagacaggat gaggatcgtt tcgcatgatt gaacaagatg
5041 gattgcacgc aggttctccg gccgcttggg tggagaggct attcggctat gactgggcac
5101 aacagacaat cggctgctct gatgccgccg tgttccggct gtcagcgcag gggcgcccgg
5161 ttctttttgt caagaccgac ctgtccggtg ccctgaatga actgcaagac gaggcagcgc
5221 ggctatcgtg gctggccacg acgggcgttc cttgcgcagc tgtgctcgac gttgtcactg
5281 aagcgggaag ggactggctg ctattgggcg aagtgccggg gcaggatctc ctgtcatctc
5341 accttgctcc tgccgagaaa gtatccatca tggctgatgc aatgcggcgg ctgcatacgc
5401 ttgatccggc tacctgccca ttcgaccacc aagcgaaaca tcgcatcgag cgagcacgta
5461 ctcggatgga agccggtctt gtcgatcagg atgatctgga cgaagagcat caggggctcg
5521 cgccagccga actgttcgcc aggctcaagg cgagcatgcc cgacggcgag gatctcgtcg
5581 tgacccatgg cgatgcctgc ttgccgaata tcatggtgga aaatggccgc ttttctggat
5641 tcatcgactg tggccggctg ggtgtggcgg accgctatca ggacatagcg ttggctaccc
5701 gtgatattgc tgaagagctt ggcggcgaat gggctgaccg cttcctcgtg ctttacggta
5761 tcgccgctcc cgattcgcag cgcatcgcct tctatcgcct tcttgacgag ttcttctgaa
5821 ttattaacgc ttacaatttc ctgatgcggt attttctcct tacgcatctg tgcggtattt
5881 cacaccgcat caggtggcac ttttcgggga aatgtgcgcg gaacccctat ttgtttattt
5941 ttctaaatac attcaaatat gtatccgctc atgagacaat aaccctgata aatgcttcaa
6001 taatagcacg tgctaaaact tcatttttaa tttaaaagga tctaggtgaa gatccttttt
6061 gataatctca tgaccaaaat cccttaacgt gagttttcgt tccactgagc gtcagacccc
6121 gtagaaaaga tcaaaggatc ttcttgagat cctttttttc tgcgcgtaat ctgctgcttg
6181 caaacaaaaa aaccaccgct accagcggtg gtttgtttgc cggatcaaga gctaccaact
6241 ctttttccga aggtaactgg cttcagcaga gcgcagatac caaatactgt tcttctagtg
6301 tagccgtagt taggccacca cttcaagaac tctgtagcac cgcctacata cctcgctctg
6361 ctaatcctgt taccagtggc tgctgccagt ggcgataagt cgtgtcttac cgggttggac
6421 tcaagacgat agttaccgga taaggcgcag cggtcgggct gaacgggggg ttcgtgcaca
6481 cagcccagct tggagcgaac gacctacacc gaactgagat acctacagcg tgagctatga
6541 gaaagcgcca cgcttcccga agggagaaag gcggacaggt atccggtaag cggcagggtc
6601 ggaacaggag agcgcacgag ggagcttcca gggggaaacg cctggtatct ttatagtcct
6661 gtcgggtttc gccacctctg acttgagcgt cgatttttgt gatgctcgtc aggggggcgg
6721 agcctatgga aaaacgccag caacgcggcc tttttacggt tcctggcctt ttgctggcct
6781 tttgctcaca tgttctt
SEQ ID NO: 5 SARS-CoV-2 Spike Antigen amino acid insert sequence of pGX9517
(IgE leader sequence underlined):
MDWTWILFLVAAATRVHSSQCVNFTTRTOLPPAYTNSFTRGVYYPDKVFRSSVLHSTQDLFLPFFSNVTWFHAI
HVSGTNGTKRFANPVLPFNDGVYFASTEKSNIIRGWIFGTTLDSKTQSLLIVNNATNVVIKVCEFQFCNDPFLG
VYYHKNNKSWMESEFRVYSSANNCTFEYVSQPFLMDLEGKQGNFKNLREFVFKNIDGYFKIYSKHTPINLVRGL
PQGFSALEPLVDLPIGINITRFQTLHISYLTPGDSSSGWTAGAAAYYVGYLQPRTFLLKYNENGTITDAVDCAL
DPLSETKCTLKSFTVEKGIYQTSNFRVQPTESIVRFPNITNLCPFGEVFNATRFASVYAWNRKRISNCVADYSV
LYNSASFSTFKCYGVSPTKLNDLCFTNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYFPLQSYGFQPTYGVGYQPYRVVVLSF
ELLHAPATVCGPKKSTNLVKNKCVNFNFNGLTGTGVLTESNKKFLPFQQFGRDIADTTDAVRDPQTLEILDITP
CSFGGVSVITPGTNTSNQVAVLYQGVNCTEVPVAIHADQLTPTWRVYSTGSNVFQTRAGCLIGAEHVNNSYECD
IPIGAGICASYQTQTNSPRRARSVASQSIIAYTMSLGVENSVAYSNNSIAIPTNFTISVTTEILPVSMTKTSVD
CTMYICGDSTECSNLLLQYGSFCTQLNRALTGIAVEQDKNTQEVFAQVKQIYKTPPIKDFGGFNFSQILPDPSK
PSKRSFIEDLLFNKVTLADAGFIKQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQDSLSSTASALGKLQDVVNQNAQAL
NTLVKQLSSNFGAISSVLNDILSRLDKVEAEVQIDRLITGRLQSLQTYVTQQLIRAAEIRASANLAATKMSECV
LGQSKRVDFCGKGYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDISGINASVVNIQK
EIDRLNEVAKNLNESLIDLQELGKYEQYIKWPWYIWLGFIAGLIAIVMVTIMLCCMTSCCSCLKGCCSCGSCCK
FDEDDSEPVLKGVKLHYT**
SEQ ID NO: 6 Nucleic acid sequence encoding SARS-CoV-2 Spike Antigen amino
acid insert sequence of pS-B.1.351/pGX9517 (IgE leader sequence
underlined):
ATGGATTGGACTTGGATTCTCTTTCTCGTTGCTGCAGCCACACGCGTTCATAGCAGCCAGTGTGTGAACTTTAC
CACCAGAACACAGCTGCCTCCTGCCTACACCAACAGCTTCACCAGAGGAGTCTACTACCCAGACAAAGTCTTCA
GAAGCTCTGTGCTGCACAGCACCCAGGACCTGTTCCTGCCTTTCTTCAGCAACGTGACCTGGTTCCACGCCATC
CACGTGTCTGGCACCAACGGCACCAAGAGATTTGCTAACCCTGTTCTTCCTTTCAATGATGGCGTGTACTTTGC
CAGCACAGAGAAGAGCAACATCATCCGAGGCTGGATCTTTGGCACCACCCTGGACAGCAAAACCCAGAGCCTGC
TGATCGTGAACAACGCCACCAACGTGGTCATCAAGGTGTGTGAGTTCCAGTTCTGCAATGACCCTTTCCTGGGC
GTGTACTACCACAAGAACAACAAGTCCTGGATGGAGTCTGAGTTCAGAGTCTACAGCTCTGCCAACAACTGCAC
ATTTGAATATGTGTCCCAGCCTTTCCTGATGGACCTGGAGGGCAAGCAGGGCAACTTTAAGAACCTGAGAGAAT
TTGTGTTCAAGAACATCGATGGCTACTTCAAGATCTACAGCAAGCACACACCCATCAACCTGGTGAGAGGCCTG
CCTCAGGGCTTCTCTGCCCTGGAGCCTCTGGTGGACCTGCCCATCGGCATCAACATCACCAGATTCCAGACCCT
GCACATCAGCTACCTGACCCCAGGAGACAGCAGCAGCGGCTGGACAGCTGGAGCTGCTGCCTACTACGTGGGCT
ACCTGCAGCCCAGGACCTTCCTGCTGAAGTACAACGAAAATGGCACCATCACAGATGCTGTTGACTGTGCCCTG
GACCCTCTTAGCGAGACCAAGTGCACCCTGAAGTCCTTCACAGTGGAGAAAGGCATCTACCAGACCAGCAACTT
CCGAGTGCAGCCAACAGAGAGCATCGTGAGATTTCCAAACATCACCAACCTGTGCCCTTTTGGAGAAGTCTTCA
ATGCCACCAGATTTGCTTCTGTGTACGCCTGGAACAGAAAAAGAATCAGCAACTGTGTGGCTGACTACTCTGTG
CTGTACAACTCTGCCTCCTTCTCCACCTTCAAGTGCTATGGAGTCTCTCCAACCAAGCTGAATGACCTGTGCTT
CACCAACGTGTATGCTGACAGCTTTGTGATCAGAGGAGATGAAGTGCGGCAGATTGCTCCTGGCCAGACAGGCA
ACATTGCTGACTACAACTACAAGCTGCCTGATGACTTCACAGGCTGTGTCATCGCCTGGAACAGCAACAACCTG
GACAGCAAGGTGGGCGGCAACTACAACTACCTGTACAGACTTTTCAGGAAGAGCAACCTGAAGCCTTTTGAAAG
AGACATCTCCACAGAGATCTACCAGGCTGGCAGCACACCCTGCAATGGTGTGAAGGGCTTCAACTGCTACTTCC
CTCTGCAGAGCTACGGCTTCCAGCCAACATATGGCGTGGGCTACCAGCCTTACAGAGTGGTGGTGCTGTCCTTT
GAGCTGCTGCACGCCCCTGCCACAGTGTGTGGCCCCAAGAAGAGCACCAACCTGGTGAAGAACAAATGTGTGAA
CTTCAATTTCAATGGCCTGACAGGCACAGGAGTGCTGACAGAGAGCAACAAGAAGTTTCTTCCTTTCCAGCAGT
TTGGAAGAGACATTGCTGACACCACAGATGCTGTGAGAGATCCTCAGACCCTGGAGATCCTGGATATCACACCC
TGCTCCTTTGGAGGAGTTTCTGTCATCACACCTGGCACCAATACCAGCAACCAAGTGGCTGTGCTGTACCAAGG
AGTGAATTGCACAGAAGTGCCTGTGGCCATCCACGCTGACCAGCTGACACCCACCTGGAGAGTGTACAGCACAG
GCAGCAATGTTTTCCAGACAAGAGCTGGCTGCCTGATTGGAGCAGAGCACGTGAACAACAGCTATGAATGTGAC
ATCCCTATTGGAGCTGGCATCTGTGCCAGCTACCAGACCCAAACCAACAGCCCAAGAAGAGCCAGATCTGTGGC
CAGCCAGAGCATCATCGCCTACACCATGAGCCTGGGAGTGGAGAACTCTGTGGCCTACAGCAACAACAGCATCG
CCATCCCCACCAACTTCACCATCTCTGTGACCACAGAGATCCTGCCTGTGTCCATGACCAAGACATCTGTGGAC
TGCACCATGTACATCTGTGGAGACAGCACAGAATGCAGCAACCTGCTGCTGCAGTACGGCTCCTTCTGCACCCA
GCTGAACAGAGCCCTGACAGGCATCGCTGTGGAGCAGGACAAGAACACACAGGAAGTGTTTGCCCAGGTGAAGC
AGATCTACAAAACACCACCCATCAAGGACTTTGGAGGCTTCAATTTCTCCCAAATCCTGCCTGACCCCAGCAAG
CCTTCCAAGAGAAGCTTCATTGAAGACCTGCTGTTCAACAAAGTGACCCTGGCTGATGCTGGCTTCATCAAGCA
GTATGGAGACTGCCTGGGAGACATTGCTGCCAGAGACCTGATCTGTGCCCAGAAGTTTAATGGCCTGACTGTGC
TGCCTCCTCTGCTGACAGATGAAATGATCGCCCAGTACACATCTGCCCTGCTGGCTGGCACCATCACCAGTGGC
TGGACATTTGGAGCTGGAGCTGCCCTGCAGATCCCTTTTGCCATGCAGATGGCCTACAGATTTAATGGCATCGG
CGTGACCCAGAACGTGCTGTACGAGAACCAGAAGCTGATCGCCAACCAGTTCAACTCTGCCATCGGCAAGATCC
AGGACAGCCTGAGCAGCACAGCCTCTGCCCTGGGCAAGCTGCAGGATGTGGTGAACCAAAACGCCCAGGCCCTG
AACACCCTGGTGAAGCAGCTGAGCAGCAACTTTGGAGCCATCTCCTCTGTGCTGAATGACATCCTGAGCCGGCT
GGACAAGGTGGAAGCAGAAGTGCAGATCGACAGACTCATCACAGGCCGCCTGCAGAGCCTGCAGACCTACGTGA
CCCAGCAGCTGATCAGAGCTGCTGAGATCCGGGCCTCTGCCAACCTGGCTGCCACCAAGATGTCAGAATGTGTG
CTGGGCCAGAGCAAAAGAGTGGACTTCTGTGGCAAAGGCTACCACCTGATGTCCTTCCCTCAGTCTGCTCCTCA
CGGCGTGGTGTTCCTGCACGTGACCTACGTGCCTGCCCAGGAGAAGAACTTCACCACAGCTCCTGCCATCTGCC
ACGATGGCAAGGCCCACTTCCCAAGAGAAGGTGTCTTTGTGTCCAATGGCACCCACTGGTTCGTGACCCAGAGA
AACTTCTACGAGCCTCAGATCATCACCACAGACAACACATTTGTGTCTGGCAACTGTGATGTGGTCATCGGCAT
CGTGAACAACACAGTTTATGACCCTCTGCAGCCTGAGCTGGACAGCTTCAAAGAAGAGCTGGACAAGTACTTCA
AGAACCACACATCTCCAGATGTGGACCTGGGAGACATCTCTGGCATCAATGCCTCTGTGGTGAACATCCAGAAG
GAAATTGACAGGCTGAACGAAGTGGCCAAGAACCTGAACGAAAGCCTCATCGACCTGCAGGAGCTGGGCAAGTA
CGAGCAGTACATCAAGTGGCCTTGGTACATCTGGCTGGGCTTCATCGCTGGCCTCATCGCCATCGTGATGGTGA
CCATCATGCTGTGCTGCATGACCAGCTGCTGCTCTTGCCTGAAGGGCTGCTGCAGCTGTGGCAGCTGCTGCAAG
TTTGATGAAGATGACTCTGAGCCTGTGCTGAAGGGCGTGAAGCTGCACTACACATGATAA
SEQ ID NO: 7 Single strand DNA sequence of pGX9517:
GCTGCTTCGCGATGTACGGGCCAGATATACGCGTTGACATTGATTATTGACTAGTTATTAATAGTAATCAATTA
CGGGGTCATTAGTTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACCTTATGGGACTTT
CCTACTTGGCAGTACATCTACGTATTAGTCATCGCTATTACCATGGTGATGCGGTTTTGGCAGTACATCAATGG
GCGTGGATAGCGGTTTGACTCACGGGGATTTCCAAGTCTCCACCCCATTGACGTCAATGGGAGTTTGTTTTGGC
ACCAAAATCAACGGGACTTTCCAAAATGTCGTAACAACTCCGCCCCATTGACGCAAATGGGCGGTAGGCGTGTA
CGGTGGGAGGTCTATATAAGCAGAGCTCTCTGGCTAACTAGAGAACCCACTGCTTACTGGCTTATCGAAATTAA
TACGACTCACTATAGGGAGACCCAAGCTGGCTAGCGTTTAAACTTAAGCTTGGTACCGAGCTCGGATCCGCCAC
CATGGATTGGACTTGGATTCTCTTTCTCGTTGCTGCAGCCACACGCGTTCATAGCAGCCAGTGTGTGAACTTTA
CCACCAGAACACAGCTGCCTCCTGCCTACACCAACAGCTTCACCAGAGGAGTCTACTACCCAGACAAAGTCTTC
AGAAGCTCTGTGCTGCACAGCACCCAGGACCTGTTCCTGCCTTTCTTCAGCAACGTGACCTGGTTCCACGCCAT
CCACGTGTCTGGCACCAACGGCACCAAGAGATTTGCTAACCCTGTTCTTCCTTTCAATGATGGCGTGTACTTTG
CCAGCACAGAGAAGAGCAACATCATCCGAGGCTGGATCTTTGGCACCACCCTGGACAGCAAAACCCAGAGCCTG
CTGATCGTGAACAACGCCACCAACGTGGTCATCAAGGTGTGTGAGTTCCAGTTCTGCAATGACCCTTTCCTGGG
CGTGTACTACCACAAGAACAACAAGTCCTGGATGGAGTCTGAGTTCAGAGTCTACAGCTCTGCCAACAACTGCA
CATTTGAATATGTGTCCCAGCCTTTCCTGATGGACCTGGAGGGCAAGCAGGGCAACTTTAAGAACCTGAGAGAA
TTTGTGTTCAAGAACATCGATGGCTACTTCAAGATCTACAGCAAGCACACACCCATCAACCTGGTGAGAGGCCT
GCCTCAGGGCTTCTCTGCCCTGGAGCCTCTGGTGGACCTGCCCATCGGCATCAACATCACCAGATTCCAGACCC
TGCACATCAGCTACCTGACCCCAGGAGACAGCAGCAGCGGCTGGACAGCTGGAGCTGCTGCCTACTACGTGGGC
TACCTGCAGCCCAGGACCTTCCTGCTGAAGTACAACGAAAATGGCACCATCACAGATGCTGTTGACTGTGCCCT
GGACCCTCTTAGCGAGACCAAGTGCACCCTGAAGTCCTTCACAGTGGAGAAAGGCATCTACCAGACCAGCAACT
TCCGAGTGCAGCCAACAGAGAGCATCGTGAGATTTCCAAACATCACCAACCTGTGCCCTTTTGGAGAAGTCTTC
AATGCCACCAGATTTGCTTCTGTGTACGCCTGGAACAGAAAAAGAATCAGCAACTGTGTGGCTGACTACTCTGT
GCTGTACAACTCTGCCTCCTTCTCCACCTTCAAGTGCTATGGAGTCTCTCCAACCAAGCTGAATGACCTGTGCT
TCACCAACGTGTATGCTGACAGCTTTGTGATCAGAGGAGATGAAGTGCGGCAGATTGCTCCTGGCCAGACAGGC
AACATTGCTGACTACAACTACAAGCTGCCTGATGACTTCACAGGCTGTGTCATCGCCTGGAACAGCAACAACCT
GGACAGCAAGGTGGGCGGCAACTACAACTACCTGTACAGACTTTTCAGGAAGAGCAACCTGAAGCCTTTTGAAA
GAGACATCTCCACAGAGATCTACCAGGCTGGCAGCACACCCTGCAATGGTGTGAAGGGCTTCAACTGCTACTTC
CCTCTGCAGAGCTACGGCTTCCAGCCAACATATGGCGTGGGCTACCAGCCTTACAGAGTGGTGGTGCTGTCCTT
TGAGCTGCTGCACGCCCCTGCCACAGTGTGTGGCCCCAAGAAGAGCACCAACCTGGTGAAGAACAAATGTGTGA
ACTTCAATTTCAATGGCCTGACAGGCACAGGAGTGCTGACAGAGAGCAACAAGAAGTTTCTTCCTTTCCAGCAG
TTTGGAAGAGACATTGCTGACACCACAGATGCTGTGAGAGATCCTCAGACCCTGGAGATCCTGGATATCACACC
CTGCTCCTTTGGAGGAGTTTCTGTCATCACACCTGGCACCAATACCAGCAACCAAGTGGCTGTGCTGTACCAAG
GAGTGAATTGCACAGAAGTGCCTGTGGCCATCCACGCTGACCAGCTGACACCCACCTGGAGAGTGTACAGCACA
GGCAGCAATGTTTTCCAGACAAGAGCTGGCTGCCTGATTGGAGCAGAGCACGTGAACAACAGCTATGAATGTGA
CATCCCTATTGGAGCTGGCATCTGTGCCAGCTACCAGACCCAAACCAACAGCCCAAGAAGAGCCAGATCTGTGG
CCAGCCAGAGCATCATCGCCTACACCATGAGCCTGGGAGTGGAGAACTCTGTGGCCTACAGCAACAACAGCATC
GCCATCCCCACCAACTTCACCATCTCTGTGACCACAGAGATCCTGCCTGTGTCCATGACCAAGACATCTGTGGA
CTGCACCATGTACATCTGTGGAGACAGCACAGAATGCAGCAACCTGCTGCTGCAGTACGGCTCCTTCTGCACCC
AGCTGAACAGAGCCCTGACAGGCATCGCTGTGGAGCAGGACAAGAACACACAGGAAGTGTTTGCCCAGGTGAAG
CAGATCTACAAAACACCACCCATCAAGGACTTTGGAGGCTTCAATTTCTCCCAAATCCTGCCTGACCCCAGCAA
GCCTTCCAAGAGAAGCTTCATTGAAGACCTGCTGTTCAACAAAGTGACCCTGGCTGATGCTGGCTTCATCAAGC
AGTATGGAGACTGCCTGGGAGACATTGCTGCCAGAGACCTGATCTGTGCCCAGAAGTTTAATGGCCTGACTGTG
CTGCCTCCTCTGCTGACAGATGAAATGATCGCCCAGTACACATCTGCCCTGCTGGCTGGCACCATCACCAGTGG
CTGGACATTTGGAGCTGGAGCTGCCCTGCAGATCCCTTTTGCCATGCAGATGGCCTACAGATTTAATGGCATCG
GCGTGACCCAGAACGTGCTGTACGAGAACCAGAAGCTGATCGCCAACCAGTTCAACTCTGCCATCGGCAAGATC
CAGGACAGCCTGAGCAGCACAGCCTCTGCCCTGGGCAAGCTGCAGGATGTGGTGAACCAAAACGCCCAGGCCCT
GAACACCCTGGTGAAGCAGCTGAGCAGCAACTTTGGAGCCATCTCCTCTGTGCTGAATGACATCCTGAGCCGGC
TGGACAAGGTGGAAGCAGAAGTGCAGATCGACAGACTCATCACAGGCCGCCTGCAGAGCCTGCAGACCTACGTG
ACCCAGCAGCTGATCAGAGCTGCTGAGATCCGGGCCTCTGCCAACCTGGCTGCCACCAAGATGTCAGAATGTGT
GCTGGGCCAGAGCAAAAGAGTGGACTTCTGTGGCAAAGGCTACCACCTGATGTCCTTCCCTCAGTCTGCTCCTC
ACGGCGTGGTGTTCCTGCACGTGACCTACGTGCCTGCCCAGGAGAAGAACTTCACCACAGCTCCTGCCATCTGC
CACGATGGCAAGGCCCACTTCCCAAGAGAAGGTGTCTTTGTGTCCAATGGCACCCACTGGTTCGTGACCCAGAG
AAACTTCTACGAGCCTCAGATCATCACCACAGACAACACATTTGTGTCTGGCAACTGTGATGTGGTCATCGGCA
TCGTGAACAACACAGTTTATGACCCTCTGCAGCCTGAGCTGGACAGCTTCAAAGAAGAGCTGGACAAGTACTTC
AAGAACCACACATCTCCAGATGTGGACCTGGGAGACATCTCTGGCATCAATGCCTCTGTGGTGAACATCCAGAA
GGAAATTGACAGGCTGAACGAAGTGGCCAAGAACCTGAACGAAAGCCTCATCGACCTGCAGGAGCTGGGCAAGT
ACGAGCAGTACATCAAGTGGCCTTGGTACATCTGGCTGGGCTTCATCGCTGGCCTCATCGCCATCGTGATGGTG
ACCATCATGCTGTGCTGCATGACCAGCTGCTGCTCTTGCCTGAAGGGCTGCTGCAGCTGTGGCAGCTGCTGCAA
GTTTGATGAAGATGACTCTGAGCCTGTGCTGAAGGGCGTGAAGCTGCACTACACATGATAACTCGAGTCTAGAG
GGCCCGTTTAAACCCGCTGATCAGCCTCGACTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGAGGATTGGGAAGACAATA
GCAGGCATGCTGGGGATGCGGTGGGCTCTATGGCTTCTACTGGGCGGTTTTATGGACAGCAAGCGAACCGGAAT
TGCCAGCTGGGGCGCCCTCTGGTAAGGTTGGGAAGCCCTGCAAAGTAAACTGGATGGCTTTCTTGCCGCCAAGG
ATCTGATGGCGCAGGGGATCAAGCTCTGATCAAGAGACAGGATGAGGATCGTTTCGCATGATTGAACAAGATGG
ATTGCACGCAGGTTCTCCGGCCGCTTGGGTGGAGAGGCTATTCGGCTATGACTGGGCACAACAGACAATCGGCT
GCTCTGATGCCGCCGTGTTCCGGCTGTCAGCGCAGGGGCGCCCGGTTCTTTTTGTCAAGACCGACCTGTCCGGT
GCCCTGAATGAACTGCAAGACGAGGCAGCGCGGCTATCGTGGCTGGCCACGACGGGCGTTCCTTGCGCAGCTGT
GCTCGACGTTGTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCTTGATCCGGCT
ACCTGCCCATTCGACCACCAAGCGAAACATCGCATCGAGCGAGCACGTACTCGGATGGAAGCCGGTCTTGTCGA
TCAGGATGATCTGGACGAAGAGCATCAGGGGCTCGCGCCAGCCGAACTGTTCGCCAGGCTCAAGGCGAGCATGC
CCGACGGCGAGGATCTCGTCGTGACCCATGGCGATGCCTGCTTGCCGAATATCATGGTGGAAAATGGCCGCTTT
TCTGGATTCATCGACTGTGGCCGGCTGGGTGTGGCGGACCGCTATCAGGACATAGCGTTGGCTACCCGTGATAT
TGCTGAAGAGCTTGGCGGCGAATGGGCTGACCGCTTCCTCGTGCTTTACGGTATCGCCGCTCCCGATTCGCAGC
GCATCGCCTTCTATCGCCTTCTTGACGAGTTCTTCTGAATTATTAACGCTTACAATTTCCTGATGCGGTATTTT
CTCCTTACGCATCTGTGCGGTATTTCACACCGCATCAGGTGGCACTTTTCGGGGAAATGTGCGCGGAACCCCTA
TTTGTTTATTTTTCTAAATACATTCAAATATGTATCCGCTCATGAGACAATAACCCTGATAAATGCTTCAATAA
TAGCACGTGCTAAAACTTCATTTTTAATTTAAAAGGATCTAGGTGAAGATCCTTTTTGATAATCTCATGACCAA
AATCCCTTAACGTGAGTTTTCGTTCCACTGAGCGTCAGACCCCGTAGAAAAGATCAAAGGATCTTCTTGAGATC
CTTTTTTTCTGCGCGTAATCTGCTGCTTGCAAACAAAAAAACCACCGCTACCAGCGGTGGTTTGTTTGCCGGAT
CAAGAGCTACCAACTCTTTTTCCGAAGGTAACTGGCTTCAGCAGAGCGCAGATACCAAATACTGTTCTTCTAGT
GTAGCCGTAGTTAGGCCACCACTTCAAGAACTCTGTAGCACCGCCTACATACCTCGCTCTGCTAATCCTGTTAC
CAGTGGCTGCTGCCAGTGGCGATAAGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCGAACGACCTACACCGAACTGAGATA
CCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCCTATGGAAAAACGCCAGCAA
CGCGGCCTTTTTACGGTTCCTGGCCTTTTGCTGGCCTTTTGCTCACATGTTCTT
SEQ ID NO: 8 pS-spike forward
ATGATCGCCCAGTACACATC,
SEQ ID NO: 9 pS-spike reverse
CACGCCGATGCCATTAAATC,
SEQ ID NO: 10 pS-spike probe
AT CACCAGTGGCTGGACATTTGGA
SEQ ID NO: 11 β-actin Forward
GTGACGTGGACATCCGTA AA;
SEQ ID NO: 12 β-actin Reverse
CAGGGCAGTAATCTCCTTCTG;
SEQ ID NO: 13 β-actin Probe
TACCCTGGCATTGCTGACAGGATG
SEQ ID NO: 14 Kozak sequence
GCCACC
```

What is claimed:

1. A nucleic acid molecule encoding a Severe Acute Respiratory Syndrome coronavirus 2 (SARS-CoV-2) spike antigen, the nucleic acid molecule comprising:
the nucleic acid sequence of nucleotides 55 to 3831 of SEQ ID NO: 2;
the nucleic acid sequence of SEQ ID NO: 2; or
the nucleic acid sequence of SEQ ID NO: 3.

2. An expression vector comprising the nucleic acid molecule according to claim 1.

3. An immunogenic composition comprising an effective amount of the expression vector according to claim 2 and a pharmaceutically acceptable excipient.

4. The immunogenic composition according to claim 3 wherein the pharmaceutically acceptable excipient comprises a buffer, optionally saline-sodium citrate buffer.

5. The immunogenic composition of claim 4, wherein the composition comprises 10 mg of the vector per milliliter of saline-sodium citrate buffer.

6. The immunogenic composition according to claim 3, further comprising an adjuvant.

7. A method of inducing an immune response against Severe Acute Respiratory Syndrome coronavirus 2 (SARS-CoV-2) in a subject in need thereof, the method comprising administering an effective amount of the immunogenic composition of claim 3 to the subject.

8. A method of protecting a subject in need thereof from infection with Severe Acute Respiratory Syndrome coronavirus 2 (SARS-CoV-2), the method comprising administering an effective amount of the immunogenic composition of claim 3 to the subject.

9. A method of treating a subject in need thereof against Severe Acute Respiratory Syndrome coronavirus 2 (SARS-CoV-2) infection, the method comprising administering an effective amount of the immunogenic composition of claim 3 to the subject, wherein the subject is thereby resistant to one or more SARS-CoV-2 strains.

10. The method of claim 7, wherein administering comprises at least one of electroporation and injection.

11. The method of claim 7, wherein administering comprises parenteral administration followed by electroporation.

12. The method of claim 7, wherein an initial dose of about 0.5 mg to about 2.0 mg of the vector is administered to the subject, optionally wherein the initial dose is 0.5 mg, 1.0 mg or 2.0 mg of the vector.

13. The method of claim 12, wherein a subsequent dose of about 0.5 mg to about 2.0 mg of the vector is administered to the subject about four weeks after the initial dose, optionally wherein the subsequent dose is 0.5 mg, 1.0 mg or 2.0 mg of the vector.

14. The method of claim 13, wherein one or more further subsequent doses of about 0.5 mg to about 2.0 mg of the vector is administered to the subject at least twelve weeks after the initial dose, optionally wherein the further subsequent dose is 0.5 mg, 1.0 mg, or 2.0 mg of the vector.

15. The method of claim 7, wherein the immunogenic composition comprises pGX9527, INO-4802 drug product or a biosimilar thereof.

16. The method of claim 7, further comprising administering to the subject at least one additional agent for the treatment of SARS-CoV-2 infection or the treatment or prevention of a disease or disorder associated with SARS-CoV-2 infection, optionally wherein the at least one additional agent comprises a SARS-CoV-2 wild-type matched vaccine, pGX9501, INO-4800 drug product or a biosimilar thereof.

17. The method of claim 16 wherein the immunogenic composition is administered to the subject before, concurrently with, or after the additional agent.

18. The method of claim 8, wherein administering comprises at least one of electroporation and injection.

19. The method of claim 8, wherein administering comprises parenteral administration followed by electroporation.

20. The method of claim 8, wherein an initial dose of about 0.5 mg to about 2.0 mg of the vector is administered to the subject, optionally wherein the initial dose is 0.5 mg, 1.0 mg or 2.0 mg of the vector.

21. The method of claim 20, wherein a subsequent dose of about 0.5 mg to about 2.0 mg of the vector is administered to the subject about four weeks after the initial dose, optionally wherein the subsequent dose is 0.5 mg, 1.0 mg or 2.0 mg of the vector.

22. The method of claim 21, wherein one or more further subsequent doses of about 0.5 mg to about 2.0 mg of the vector is administered to the subject at least twelve weeks after the initial dose, optionally wherein the further subsequent dose is 0.5 mg, 1.0 mg, or 2.0 mg of the vector.

23. The method of claim 8, wherein the immunogenic composition comprises pGX9527, INO-4802 drug product or a biosimilar thereof.

24. The method of claim 8, further comprising administering to the subject at least one additional agent for the treatment of SARS-CoV-2 infection or the treatment or prevention of a disease or disorder associated with SARS-CoV-2 infection, optionally wherein the at least one additional agent comprises a SARS-CoV-2 wild-type matched vaccine, pGX9501, INO-4800 drug product or a biosimilar thereof.

25. The method of claim 24 wherein the immunogenic composition is administered to the subject before, concurrently with, or after the additional agent.

26. The method of claim 9, wherein administering comprises at least one of electroporation and injection.

27. The method of claim 9, wherein administering comprises parenteral administration followed by electroporation.

28. The method of claim 9, wherein an initial dose of about 0.5 mg to about 2.0 mg of the vector is administered to the subject, optionally wherein the initial dose is 0.5 mg, 1.0 mg or 2.0 mg of the vector.

29. The method of claim 28, wherein a subsequent dose of about 0.5 mg to about 2.0 mg of the vector is administered to the subject about four weeks after the initial dose, optionally wherein the subsequent dose is 0.5 mg, 1.0 mg or 2.0 mg of the vector.

30. The method of claim 29, wherein one or more further subsequent doses of about 0.5 mg to about 2.0 mg of the vector is administered to the subject at least twelve weeks after the initial dose, optionally wherein the further subsequent dose is 0.5 mg, 1.0 mg, or 2.0 mg of the vector.

31. The method of claim 9, wherein the immunogenic composition comprises pGX9527, INO-4802 drug product or a biosimilar thereof.

32. The method of claim 9, further comprising administering to the subject at least one additional agent for the treatment of SARS-CoV-2 infection or the treatment or prevention of a disease or disorder associated with SARS-CoV-2 infection, optionally wherein the at least one additional agent comprises a SARS-CoV-2 wild-type matched vaccine, pGX9501, INO-4800 drug product or a biosimilar thereof.

33. The method of claim 32 wherein the immunogenic composition is administered to the subject before, concurrently with, or after the additional agent.

34. A Severe Acute Respiratory Syndrome coronavirus 2 (SARS-CoV-2) spike antigen comprising:
the amino acid sequence of residues 19 to 1277 of SEQ ID NO: 1; or
the amino acid sequence of SEQ ID NO: 1.
